(19) United States
(12) Patent Application Publication
(10) Pub. No.: US 2006/0281763 A1
(43) Pub. Date: Dec. 14, 2006

(54) Carboxamide inhibitors of TGFbeta

(76) Inventors: Jonathan R. Axon, Milpitas, CA (US); Sarvajit Chakravarty, Mountain View, CA (US); Barry Hart, Palo Alto, CA (US); Glenn McEnroe, San Mateo, CA (US); Alison Murphy, Milpitas, CA (US); Karen Pontius, Aromas, CA (US); Peijue Sheng, San Jose, CA (US); Xiaojing Wang, Foster City, CA (US); Shanthi Yellapregada, Milpitas, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/390,980

(22) Filed: Mar. 27, 2006

Related U.S. Application Data
(60) Provisional application No. 60/665,095, filed on Mar. 25, 2005.

(51) Int. Cl. C07D 403/14; C07D 403/02; A61K 31/506

(52) U.S. Cl. 514/256; 544/328

(57) ABSTRACT

Certain appropriately substituted forms of pyrimidine having a pyridylamine group at C-4 of the pyrimidine and an amide group on the pyridine ring are useful in the treatment of conditions associated with excessive TGFβ activity.

### RELATED APPLICATIONS

The application claims priority to U.S. Provisional Patent Application No. 60/665,095, filed Mar. 25, 2005, which is hereby incorporated by reference in its entirety.

### FIELD OF THE INVENTION

The invention relates to methods of treating various disorders associated with excessive activity of transforming growth factor beta (TGF-beta, or TGFβ). More specifically, it concerns certain amide-substituted pyrimidine compounds having a 4-pyridylamine group at C-4 that are useful in these methods.

### BACKGROUND ART

Transforming growth factor-beta (TGFβ) denotes a superfamily of proteins that includes, for example, TGFβ 1, TGFβ 2, and TGFβ 3, which are pleiotropic modulators of cell growth and differentiation, embryonic and bone development, extracellular matrix formation, hematopoiesis, immune and inflammatory responses (Roberts and Sporn Handbook of Experimental Pharmacology (1990) 95:419-58; Massague, et al., Ann. Rev. Cell. Biol. (1990) 6:597-646). Other members of this superfamily include activin, inhibin, bone morphogenic protein, and Mullerian inhibiting substance. The members of the TGFβ family initiate intracellular signaling pathways leading ultimately to the expression of genes that regulate the cell cycle, control proliferative responses, or relate to extracellular matrix proteins that mediate outside-in cell signaling, cell adhesion, migration and intercellular communication.

Therefore, inhibitors of the TGFβ intracellular signaling pathway are useful treatments for fibroproliferative diseases. Specifically, fibroproliferative diseases include kidney disorders associated with unregulated TGFβ activity and excessive fibrosis including glomerulonephritis (GN), such as mesangial proliferative GN, immune GN, and crescentic GN. Other renal conditions include diabetic nephropathy, renal interstitial fibrosis, renal fibrosis in transplant patients receiving cyclosporin, and HIV-associated nephropathy. Collagen vascular disorders include progressive systemic sclerosis, polymyositis, scleroderma, dermatomyositis, eosinophilic fascitis, morphea, or those associated with the occurrence of Raynaud's syndrome. Lung fibroses resulting from excessive TGFβ activity include adult respiratory distress syndrome, chronic obstructive pulmonary disease (COPD), idiopathic pulmonary fibrosis, and interstitial pulmonary fibrosis often associated with autoimmune disorders, such as systemic lupus erythematosus and scleroderma, chemical contact, or allergies. Another autoimmune disorder associated with fibroproliferative characteristics is rheumatoid arthritis.

Fibroproliferative conditions can be associated with surgical eye procedures. Such procedures include retinal reattachment surgery accompanying proliferative vitreoretinopathy, cataract extraction with intraocular lens implantation, and post glaucoma drainage surgery.

In addition, members of the TGFβ family are associated with the progression of various cancers. M. P. de Caestecker, E. Piek, and A. B. Roberts, J. National Cancer Inst., 92(17), 1388-1402 (2000). For example, it has been found that TGFβ 1 inhibits the formation of tumors, probably by inhibition of the proliferation of nontransformed cells. However, once a tumor forms, TGFβ1 promotes the growth of the tumor. N. Dumont and C. L. Arteaga, Breast Cancer Res., Vol. 2, 125-132 (2000). Thus inhibitors of the TGFβ pathway are also useful for the treatment of many forms of cancer, such as lung cancer, skin cancer, and colorectal cancer. In particular, they are useful to treat cancers of the breast, pancreas, and cancers of the brain, such as glioma.

The compounds of the invention herein are derivatives of pyrimidine. PCT publication WO01/47921 describes pyrimidine and triazine compounds that are inhibitors of kinase activities associated with various inflammatory conditions, as opposed to the treatment of fibroproliferative disorders described herein. The above mentioned PCT publication describes the use of the compounds disclosed only for treatment of the inflammatory aspects of certain autoimmune diseases. Further, the compounds described differ from those described herein by virtue of the substitutions required on the pyrimidine nucleus; among other distinctions, the compounds disclosed in the PCT publication do not include phenyl bound directly to the pyrimidine ring.

Related compounds, some of which have the 4-pyridylamine group at C-4 on the pyrimidine, are disclosed in published U.S. Patent Applications, publications no. US 2004-0132730 A1, US 2004-0132159-A1 and US 2005/0004143-A1. Those applications, however, disclose a preference for certain electron-donating substituents on the pyridine ring of the 4-pyridylamine group, including alkyl, amine and alkoxy groups, without disclosing a preferred position for those substituents, or they suggest a variety of aryl groups which may be pyridyl for the 4-position substituent on a pyrimidine ring but do not disclose or suggest the combination of features of the present invention, in particular they do not suggest the amides of the present invention. The present invention provides compounds specifically including a 4-pyridylamine that is substituted by a carboxamide group which is attached at position 3 on the pyridine ring. The carboxamide is attached via its carbonyl carbon, and is typically a secondary amide; furthermore, the compounds of the present invention include specific functional groups and substituents particularly on the amide group, that are selected for their ability to reduce metabolism and increase bioavailability of the active species.

U.S. Pat. No. 6,476,031 ('031) also discloses compounds containing a quinazoline ring linked to an aryl group at C-4 of the quinazoline. The compounds are reported to act at the TGFβ site, and some of the compounds include a 4-pyridylamine group at C-4 of the quinazoline. However, the '031 patent discloses that the aryl group linked to C-4 of the quinazoline is preferably unsubstituted 4-pyridyl, and it does not disclose any compounds where the 4-pyridyl includes an amide substituent such as the ones at the 3-position of the 4-pyridyl group in the compounds of the present invention.

### DISCLOSURE OF THE INVENTION

The invention is directed to methods, compositions, and compounds useful in treating conditions that are characterized by excessive TGFβ activity. These conditions are, most prominently, fibroproliferative diseases. However, the conditions for which the compounds and methods are useful include any medical condition characterized by an undesirably high level of TGFβ activity.

The compounds of the invention have been found to inhibit TGFβ and are thus useful in treating diseases mediated by the activity of this family of factors. Moreover, they have been designed to incorporate certain features that enhance their ability to exhibit activity in cellular systems: they incorporate structural features that improve water solubility or metabolic stability, and thus their intrinsic TGFβ activity is better translated into efficacy in a cell. The compounds of the invention are of formula (1): 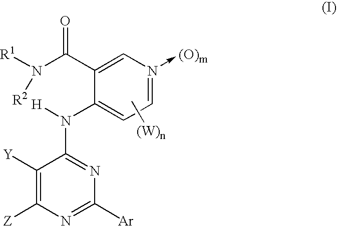
wherein Ar represents an optionally substituted phenyl ring;
Y represents H, halo, NO₂, or an optionally substituted member selected from the group consisting of alkyl, alkenyl, alkynyl, heteroalkyl, heteroalkenyl, heteroalkynyl, aryl, heteroaryl, arylalkyl, heteroarylalkyl, acyl, and heteroacyl,
   or Y can be NR₂, wherein each R is independently H or an optionally substituted alkyl, alkenyl, alkynyl, acyl, aryl or arylalkyl group or a heteroform of any of these groups, and wherein two R groups can cyclize to form an optionally substituted 3-8 membered heterocyclic ring;
R¹ represents an optionally substituted group selected from alkyl, heteroalkyl, acyl, alkoxy, alkylamino, heteroacyl, aryl, heteroaryl, arylalkyl, and heteroarylalkyl, where each heteroalkyl, heteroacyl, heteroaryl, and heteroarylalkyl includes one or more heteroatoms selected from O, N, S and P,
   provided that R¹ is not a group of the formula —CH₂—CH(OH)—R⁴, where R⁴ is H or an optionally substituted hydrocarbyl group that does not comprise an amine;
R² represents H, or R² represents CH₂ and R¹ and R² cyclize to form an optionally substituted piperidine, morpholine, or piperazine ring, or a pyrrolidine ring substituted with at least one amino or halo substituent;
Z represents H, halo, NO₂, or an optionally substituted member selected from the group consisting of alkyl, alkenyl, alkynyl, heteroalkyl, heteroalkenyl, heteroalkynyl, aryl, heteroaryl, arylalkyl, heteroarylalkyl, acyl, and heteroacyl, or Z is NR₂,
   wherein each R is independently H or an optionally substituted alkyl, alkenyl, alkynyl, acyl, heteroacyl, aryl or arylalkyl group or a heteroform of any of these groups;
each W independently represents halo, NR₂, NO₂, CN, CF₃, or an optionally substituted member selected from the group consisting of alkyl, alkenyl, alkynyl, heteroalkyl, heteroalkenyl, heteroalkynyl, aryl, heteroaryl, acyl, heteroacyl, arylalkyl, and heteroarylalkyl,
   wherein each R is independently H or an optionally substituted alkyl, alkenyl, alkynyl, acyl, aryl, heteroalkyl or heteroaryl group;
   m is 0 or 1;
   n is 0-3;
   and
      (a) Y is selected from the group consisting of a 5-6 membered cyclic amine, OH, F, Cl, Br, and I; or
      (b) m is 1; or
      (c) R¹ is OH or an optionally substituted alkoxy or an optionally substituted alkylamine, or
      (d) R² represents CH₂ and R¹ and R² cyclize to form an optionally substituted piperidine, morpholine, or piperazine ring, or a pyrrolidine ring substituted with at least one amino or halo substituent;
      (e) R¹ comprises C—NH₂, a nitrile, a lactam or a lactone ring, or a ketone, or an optionally substituted 4-5 membered cyclic amine; or
      (f) R¹ comprises at least two substructures independently selected from the group consisting of:
         (1) C—NH—C,
         (2) C—OH,
         (3) C═O,
         (4) P═O,
         (5) S═O,
         (6) C═N,
         (7) a non-cyclic ether oxygen,
         (8) a tertiary non-acylated amine;
         (9) a 5-6 membered aromatic or heteroaromatic ring,
         (10) C—X where X is selected from OH, Cl, and F,
         (11) C_{T}—O—R⁴, wherein C_{T} represents a carbon bonded to three other carbon atoms, and R⁴ is H or an optionally substituted hydrocarbyl group, and
         (12) an optionally substituted 3 to 8 membered carbocyclic ring; or
      (f) R¹ comprises —(CH₂)₃—OR⁴ or —CH₂)₃—N(R⁴)₂, wherein each R⁴ is independently H or an optionally substituted hydrocarbyl group;
or a pharmaceutically acceptable salt thereof.

The invention is also directed to pharmaceutical compositions containing one or more compounds of formula (I) or their pharmaceutically acceptable salts, including certain prodrug forms of such compounds, as active ingredients, and to methods of treating conditions characterized by an excessive level of TGFβ activity or fibroproliferative conditions or cancers using these compounds and compositions.

### MODES OF CARRYING OUT THE INVENTION

The compounds of formula (I) are useful in treating conditions which are characterized by an excessive level of TGFβ activity. As used herein, “TGFβ” refers to the superfamily which includes TGFβ1, TGFβ2, and TGFβ3 as well as other members of the family known or which become known in the art such as inhibin, bone morphogenic protein, and the like. One or more of these family members may be more active than desired in the conditions which the compounds of the invention are designed to ameliorate or prevent.

Conditions “characterized by an excessive level of TGFβ activity” include those wherein TGFβ synthesis is stimulated so that TGFβ is present in enhanced amount, and those wherein TGFβ latent protein is undesirably activated or converted to active TGFβ protein, and those wherein TGFβ receptors are upregulated, and those wherein the TIFF protein shows enhanced binding to cells or extracellular matrix in the location of the disease. Thus, in either case, “excessive level of TGFβ activity” refers to any condition wherein the activity of TGFβ is undesirably high, regardless of the cause and regardless of whether the actual amount or activity of TGFβ present is within a ‘normal’ range.

As used herein the term “hydrocarbyl” refers to a C1-C20 hydrocarbon group that may contain alkyl chains, rings, or combinations of chains and rings, and may contain one or more unsaturated and/or aromatic structures, but which contains no heteroatoms unless it is substituted. A hydrocarbyl group may be substituted at any available position with suitable substituents as further described herein.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., either as 1-10C or as C1-C10 when the group can contain up to ten carbon atoms. When heteroatoms (N, O and S typically) are allowed to replace carbon atoms as in heteroalkyl groups, for example, the numbers describing the group represent the sum of the number of carbon atoms in the group plus the number of such heteroatoms that are included as replacements for carbon atoms.

Typically, the alkyl, alkenyl and alkynyl substituents of the invention contain 1-10C (alkyl) or 2-1° C. (alkenyl or alkynyl). Preferably they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl.

“Heteroalkyl”, “heteroalkenyl”, and “heteroalkynyl” are defined similarly to the corresponding hydrocarbyl (alkyl, alkenyl and alkynyl) groups, but the ‘hetero’ terms refer to groups that contain 1-3 O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical and preferred sizes for heteroforms of alkyl, alkenyl and alkynyl groups are the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including halo, C1-C8 alkyl, C2-C8 alkenyl, C2-C8 alkynyl, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and preferably n is 1-4, though where specified, an alkylene can also be substituted by other groups, and can be of other lengths, and the open valences need not be at opposite ends of a chain. Thus —CH(Me)— and —C(Me)₂— may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group.

“Optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced accordingly.

“Halo”, as used herein includes fluoro, chloro, bromo and iodo. Fluoro and chloro are often preferred.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

The Invention Compounds

The compounds useful in the invention are derivatives of pyrimidine containing mandatory substituents at positions corresponding to the 2- and 4-positions of the pyrimidine ring. The compounds include a 4-pyridylamine group at position 4 of the pyrimidine ring and a phenyl group at position 2 of the pyrimidine ring; each of these may be substituted. Optionally, the 4-pyridyl group may be a pyridine-N-oxide.

The compounds further include an amide group that is attached to the pyridyl ring at its position 3; this amide group is connected to the pyridyl ring through its carbonyl carbon. The nitrogen of the amide may have one hydrogen and one non-hydrogen substituent, R¹, attached to it, or it may be part of a ring formed by cyclizing R¹ onto a CH₂ group represented by R². Accordingly, the compounds all share a common skeleton, and differ in the nature of certain optional substituents on the aryl rings and on the nitrogen of the carboxamide shown in formula (1).

The substituent R¹ of this carboxamide may be selected to avoid certain metabolic pathways that have been found to reduce the activity of certain compounds previously reported. Similarly, the substituents on R¹ may be selected to promote water solubility and bioavailability.

For example, it has been found that if the amide in compounds related to the compound of formula (1) is of the form C(═O)—NH—CH₂—CH(OH)—R, the secondary hydroxyl in this amide group is readily oxidized in vivo. Accordingly, the present invention provides compounds less prone to such oxidation, such as compounds that incorporate an additional substituent on the portion of the amide containing this hydroxyl, in order to prevent or slow such oxidative metabolism. For example, by making the secondary alcohol into a tertiary alcohol, its oxidation is prevented. Alternatively, additional substituents may be placed around the hydroxyl-bearing carbon to slow the oxidative process, as in C(═O)—NH—CHR′—CH(OH)—R, where the added R′ is positioned to sterically slow down that oxidation. In other examples, the secondary hydroxyl is modified into an ether or an ester or a phosphate ester; these compounds are often intrinsically active on their own and are thus useful as TGFβ inhibitors, and they may also serve as pro-drugs of the secondary alcohol. Such prodrugs can prolong delivery of the secondary alcohol as active TGFβ inhibitors by releasing the alcohol compound gradually in vivo as the prodrug undergoes metabolic cleavage to the free secondary alcohol, such as by ester or phosphate ester hydrolysis.

Similary, the addition of hydrogen bond accepting groups in R¹, such as C═O, S═O, P═O, C═N, C≡N, certain ether oxygens, and tertiary amines that are not acylated so they retain some basicity, can be employed to increase bioavailability, possibly by increasing the tendency of this part of the molecule to partition into an aqueous phase. Likewise, certain hydrogen bond donor substructures such as —OH and NH also can increase the effectiveness of the compounds of the invention, and are often suitably incorporated into the R¹ group of the amide in compounds of formula (1). Moreover, the incorporation of two such substructures into R¹ can enhance the activity of the compounds. Accordingly, in certain embodiments, the compounds of formula (1) include at least two substructures in R¹ that are selected from C—NH—C, C—OH, C═O, P═O, S═O, ═N, a non-cyclic ether oxygen, a tertiary non-acylated amine, a 5-6 membered aromatic or heteroaromatic ring, certain optionally substituted cyclic amines, C—X where X is selected from Cl, F and CN, and an oxygen bonded to a tertiary carbon, of the formula C_{T}—O—R⁴, where R⁴ is H or an optionally substituted hydrocarbyl group, and C_{T} represents a carbon bonded to three other carbon atoms. Likewise the activity of the TGFβ compounds can be improved by certain substituents on the pyrimidine ring at position 5 (represented by the Y group), including halo (F, Cl, Br or I), cyclic amines having 5-8 ring members which may be connected to the pyrimidine ring by the amine nitrogen or by a ring carbon, or —OH. In other embodiments, R¹ comprises a lactam or lactone ring, or a ketone carbonyl. Preferably, the amide group containing R¹ is not of the formula C(═O)—NH—CH₂—CH(OH)—R⁴, where R⁴ is H or an optionally substituted hydrocarbyl group that does not contain an amine, which substructure appears to facilitate oxidative metabolic degradation.

As described above, R¹ can be selected to improve bioavailability of the compounds of the invention, and in many embodiments it includes one or more polar functional groups such as those listed above. It may comprise an aromatic ring; however, in many embodiments where it represents an aryl or heteroaryl group, that group is a polar ring such as a phenyl substituted with an amide group, or a heteroaryl group such as a pyrrole or imidazole ring, or a cyclic amine. In other embodiments, R¹ incorporates one or more halo substituents on an alkyl group, such as for example a trifluoromethyl, which can improve water solubility and also deter metabolism.

In some embodiments, R¹ is hydroxyl or an alkoxy or heteroalkoxy, or a substituted amine group, with O or N directly bonded to the carboxamide nitrogen to form an acyl hydrazide or a hydroxamate derivative; an optionally substituted C1-C8 alkoxy or C1-C8 heteroalkoxy is sometimes preferred. In other embodiments, R¹ is an optionally substituted alkyl, heteroalkyl, acyl, heteroacyl, aryl, arylalkyl, heteroaryl, or heteroarylalkyl group. Typically, R¹ is C1-C8 alkoxy, substituted amino, C1-C8 alkyl, C2-C8 heteroalkyl, C6-C10 aryl, C5-C10 heteroaryl, C7-C12-arylalkyl, or C6-C12 heteroarylalkyl, where each of the foregoing groups is optionally substituted by the substituents described herein as suitable for such groups. In many embodiments, R¹ is an optionally substituted C1-C8 alkyl or C1-C8 heteroalkyl group, which can be or include a cyclic group, that contains at least one and preferably two groups selected from those mentioned above, i.e., C—NH—C, C—OH, C═O, P═O, S═O, ═N, a non-cyclic ether oxygen, a tertiary non-acylated amine, a 5-6 membered aromatic or heteroaromatic ring, C—X where X is selected from Cl, F and CN, and an oxygen bonded to a tertiary carbon, of the formula C_{T}—O—R⁴, where R⁴ is H or an optionally substituted hydrocarbyl group, and C_{T} represents a carbon bonded to three other carbon atoms. In some embodiments, R¹ includes a heterocyclic group having 3-8 ring members, at least one of which is a heteroatom selected from N, O and S; furanose and pyranose rings are sometimes included, and at other times a lactam, lactone, or 5-6 membered nonaromatic ring containing a nitrogen atom is included.

Preferred substituents for the groups comprising R¹ include hydroxyl, halo especially F or C1, C1-C8 alkoxy, C1-C8 alkyl, C2-C8 heteroalkyl, CN, mono- and di-(C1-C8)-alkyl amines, —C(═O)R, COOR, CONR₂, —NC(O)R, —C(O)NR₂, —NRC(O)OR, SO₂R, SO₂NR₂, —OP(═O)(OR)₂, and, where available valences permit, ═O, ═N—OH, ═N—(C1-C8 alkyl), and ═N—(C2-C8-heteroalkyl). Each R in these substituents is independently H, C1-C8 alkyl, C2-C8 heteroalkyl, C6-C10 aryl, C5-C10 heteroaryl, C1-C8 acyl or C2-C8 heteroacyl. Preferred embodiments of R¹ include H, OR, NHR, C1-C8 alkyl and C2-C8 heteroalkyl, wherein each R represents H or C1-C8 alkyl or C2-C8 heteroalkyl, and each alkyl or heteroalkyl is optionally substituted as just described.

In certain embodiments, R¹ is of the form R₂—C(OH)—CH₂— or R—CH(OH)—CHR— or HO—CH₂—CHR—, where each R is independently a C1-C8 alkyl or heteroalkyl group and may be substituted, and where two R groups can cyclize together to form a 3-8 membered ring that can include up to two heteroatoms selected form N, O and S as ring members. These embodiments are distinguished from compounds having R¹═R—CH(OH)—CH₂— because the additional R groups are positioned to slow oxidative metabolism that has been shown to occur with the latter group; thus these embodiments of R¹ promote the desired biological activity of the compound of formula (1).

In some embodiments, R¹ comprises a substituted alkyl or heteroalkyl group that is preferably cyclic and is linked to the amide nitrogen through an aminoalkylene group such as —NR—(CH₂)₂₋₄[N], where [N] represents the nitrogen of the carboxamide shown in formula (1). R in this linkage can be H or C1-C4 alkyl or heteroalkyl, which can be substituted with, for example, ═O. In such embodiments, R¹ can include in addition to the linkage —NR—(CH₂)₂₋₄[N], a pyranose or furanose ring, which may be substituted and is in some instances substituted by one or more hydroxyl groups, preferably 2-4 hydroxyl groups, and which is either bonded directly to N of the linkage, or connected to that nitrogen by an optionally substituted C1-C4 alkylene or heteroalkylene linker such as (CH₂)₂₋₃ or —O(CH₂)₁₋₃, each of which can be substituted. In some such embodiments, this alkylene or heteroalkylene linker is substituted with one or two substituents such as, but not limited to, hydroxyl, ═O, or C1-C4 alkyl. In other such embodiments, R¹ can comprise an aryl, heteroaryl, carbocyclic, or heterocyclic ring Rₙ having 3-8 ring members, up to two of which can be heteroatoms selected from N, O and S, that is linked to the carboxamide of formula (1) through the above described aminoalkylene linker, e.g., Rₙ—(CH₂)₀₋₂—NR—(CH₂)₂₋₄[N]. In such embodiments, the ring Rₙ or the linker connecting Rₙ to the carboxamide nitrogen can include one or more ether linkages or be substituted with one or more substituents such as halo, hydroxyl, or C1-C4 alkoxy or an amino, C1-C4 alkylamino, or di-(C1-C4 alkyl)amino group.

In other embodiments where R¹ comprises a linking aminoalkylene group such as —NR—(CH₂)₂₋₄[N] as described above bonded to the carboxamide nitrogen, R¹ further comprises an acyl group such as RC(═O)—, RO—C(═O)—, or R₂N—C(═O)—, where each R independently represents H or an optionally substituted C1-C4 alkyl or heteroalkyl group. In such embodiments, R¹ can take the form R-Q-C(═O)—NR—(CH₂)₂₋₄[N], for example, where Q represents a bond, O or NR, and each R independently represents H or an optionally substituted C1-C4 alkyl or heteroalkyl group. Similarly, R1 can comprise a sulfonayl, guanidinyl, or cyanoguanidinyl group attached through —NR—(CH₂)₂₋₄[N] as described above for the acyl groups.

In other embodiments where R¹ is linked to the carboxamide nitrogen through an aminoalkylene group such as —NR—(CH₂)₂₋₄[N] as described above, R¹ comprises a halogenated C1-C8 alkyl or heteroalkyl such as a polyfluorinated C1-C4 alkyl group, which can promote water solubility and slow metabolism. Specific examples of such embodiments include compounds having a group such as CF₃CF₂(CH₂)₀₋₃—NR—(CH₂)₂₋₄[N] as R¹.

In other embodiments, R¹ comprises a lactam, lactone, or heterocyclic ring such as a 5-6 membered cyclic ether or acyclic amine having 4-5 ring members, each of which is optionally substituted with one or more substituents that can promote bioavailability, such as C1-C4 alkoxy, ═O, halo such as one or more fluoro substituents, or CN, or with two or more hydroxyl substituents. In certain embodiments, R¹ is a dicarbonyl group such as RO—C(═O)—C(═O)— or R₂N—C(═O)—C(═O)—, where each R is independently H or C1-C8 optionally substituted alkyl or heteroalkyl group, or an optionally substituted C5-C12 aryl, arylalkyl, heteroaryl, or heteroarylalkyl group.

In certain embodiments, R¹ comprises (CH₂)₃—OR⁴ or (CH₂)₃—N(R⁴)₂, which positions the oxygen or nitrogen at an ideal distance from the pyridyl ring to allow it to increase affinity for the TGFβ binding pocket. In these embodiments, each R⁴ can be H or an optionally substituted C1-C20 hydrocarbyl group. Preferably, each R⁴ is H or a C1-C4 alkyl, or N(R⁴)₂ represents a 4-7 membered cyclic amine having up to two substituents suitable for an alkyl group and optionally including one additional heteroatom selected from N, O and S.

Other substituents may also be included on the pyrimidine, pyridine and aryl rings; in particular, the phenyl ring represented by Ar is optionally substituted with the groups described herein as suitable for placement on an aryl or heteroaryl ring, and may be substituted with 1-2 substituents selected from C1-C4 alkyl, C1-C4 alkoxy, CF₃, halo, and CN in certain embodiments.

The pyridyl ring (which may be referred to as a nicotinamide, due to the presence of the 3-position amide group) can be substituted with up to three substituents suitable for placement on an aryl ring, so n can be 0-3. Preferably, n is 0 or 1 in formula (1). In certain embodiments the pyridyl ring of formula (1) is substituted with one group selected from C1-C4 alkyl, C1-C4 alkoxy, CF₃, halo, and CN, and preferably selected from halo, methyl, CF₃, and OMe. In other embodiments, the pyridyl ring is not substituted other than by the amide shown in formula (1), i.e., n is 0.

Typical embodiments of W include the substituents described herein as substituents for an aryl group generally. These include including halo, R, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with the same groups that may be present as substituents on the aryl group. Preferred embodiments for W include halo and CN, as well as CF₃, R, OR, SR, and NR₂, wherein each R is independently H or C1-C6 alkyl optionally substituted with ═O or any of the substituents that can comprise W.

The pyrimidine ring may also be substituted with groups Y and Z at positions 5 and 6; these substituents are selected from those described herein as suitable for attachment to an aryl ring, and at least one such group is typically present, particularly at position 5.

Substituents represented by Y and Z, include, but are not limited to, alkyl, alkenyl, alkynyl, acyl, aryl, heteroalkyl, heteroalkenyl, heteroalkynyl, heteroacyl, arylalkyl, and heteroarylalkyl, each of which is optionally substituted by halo, ═O (where two available valences are on a single atom), R, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R other than H is optionally substituted with the same groups that may be present as substituents on an aryl group. Additionally, Y and Z may independently be H, halo, OR, NR₂, SR, —SOR, —SO₂R, —OCOR, —NRCOR, —NRCONR₂, —NRCOOR, —OCONR₂, —COOR, SO₂R, NRSOR, NRSO₂R, —SO₃R, —CONR₂, SO₂NR₂, —CN, —CF₃, or NO₂, wherein each R is independently H, (1-8C) alkyl, (1-8C) heteroalkyl, (1-8C) acyl, (1-8C) heteroacyl, C6-C10 aryl, or C5-C10 heteroaryl and each R is optionally substituted with the same groups described above as suitable substituents for each group that comprises R.

Preferably, Y is not H, so position 5 of the pyrimidine ring is generally substituted. In certain embodiments, Y is selected from halo, OH, OR, NR₂, and R, wherein each R is an optionally substituted group selected from C1-C8 alkyl, C1-C8 heteroalkyl, C6-C12 arylalkyl, and C6-C12 heteroarylalkyl, and where two R groups of NR₂ can optionally cyclize to form 3-8 membered ring containing 1-2 heteroatoms selected from N, O and S. Preferred embodiments of Y include methoxy, ethoxy, propoxy, and isopropoxy; dimethylamino, pyrrolidin-1-yl, piperidine-1-yl, and morpholin-4-yl; and methyl, ethyl, propyl, isopropyl, cyclopropyl, t-butyl, cyclobutyl, and cyclopentyl.

Position 6 of the pyrimidine can also be substituted, so that Z can represent a substituent such as halo, NO₂, or an optionally substituted member selected from the group consisting of alkyl, alkenyl, alkynyl, heteroalkyl, heteroalkenyl, heteroalkynyl, aryl, heteroaryl, arylalkyl, heteroarylalkyl, acyl, and heteroacyl, or Z is NR₂, wherein each R is independently H or an optionally substituted alkyl, alkenyl, alkynyl, acyl, heteroacyl, aryl or arylalkyl group or a heteroform of any of these groups. While position 6 of the pyrimidine can be so substituted, in many embodiments it is unsubstituted, i.e., Z represents H.

Ar represents an optionally substituted phenyl; in many embodiments, Ar represents phenyl that is substituted with at least one and preferably two or more substituents selected from the group consisting of halo, CN, CF₃, R, OR, NO₂, SR, SO₂R, NR₂, and acyl, where each R is independently H, C1-C6 alkyl, C1-C8 acyl, or aryl. In many embodiments, Ar is substituted with at least one halo, and in certain embodiments it is substituted with 1-2 groups selected from C1-C4 alkyl, C1-C4-alkoxy, CF₃, CN and halo; halo in such embodiments is sometimes preferably Cl or F. Certain embodiments of Ar include phenyl substituted with F or Cl ortho to the carbon through which the phenyl is linked to the pyrimidine ring, which is referred to as position 2 for convenient reference. In some such embodiments, Ar further comprises a second substituent which may also be halo at position 5. A preferred embodiment for Ar, which may be combined with the preferred features of each of the other structural components of the compound of formula (1), has F or Cl at position 2 and Cl or F at position 5 of the phenyl ring.

As stated above, any aryl, alkyl, heteroaryl, heteroalkyl, acyl, heteroacyl, arylalkyl, or heteroarylalkyl group included within a substituent may itself be substituted with the substituents typical for such groups. These substituents may occupy all available positions of the group, preferably 1-2 positions, or more preferably only one position.

Where any of the aryl moieties, including those depicted in formula (I) especially the phenyl moieties, is described as optionally containing at least two substituents, if those substituents can occupy adjacent positions on the aryl ring, they may, when taken together, form a 5-7 membered carbocyclic or heterocyclic ring. Examples of such rings include dioxolane fused onto a phenyl ring, or oxazole fused to a pyridine ring.

The compounds of formula (I) may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, citric, alkylsulfonic, arylsulfonic, and glucuronic acids and the like. If a carboxyl moiety is present on the compound of formula (1), the compound may also be supplied as a salt with a pharmaceutically acceptable cation, such as sodium, potassium, or an ammonium salt.

The compounds of formula (I) may also be supplied in the form of a “prodrug” which is designed to release the compound of formula (1) when administered to a subject. Prodrug designs are well known in the art, and depend on the substituents contained in the compound of formula (1). For example, a substituent containing sulfhydryl could be coupled to a carrier which renders the compound biologically inactive until removed by endogenous enzymes or, for example, by enzymes targeted to a particular receptor or location in the subject. Similarly, ester and amide linkages may be employed to mask hydroxyl, amino, or carboxyl groups on an active molecule within the scope of the invention, and such groups may be enzymatically cleaved in vivo to release the active molecule.

In particular prodrug embodiments, compounds having a hydroxyl group on R¹ are sometimes acylated or phosphorylated with groups that can be hydrolyzed under physiological conditions at an appreciable rate. Suitable acyl groups include C1-C8 acyl groups, which may be substituted, and which can include cyclic and/or aryl groups; for example, benzoyl, acetyl, formyl, and methoxyacetyl esters of a hydroxyl group in R¹ are all suitable prodrugs. Similaryl, the phosphate esters of hydroxyl groups on R¹ are suitably used as prodrugs, including the mono- and di- and tri-alkyl esters. One of the esterified groups is of course on R1, and the other ester(s) may be formed with a C1-C4 alcohol (e.g., they can include methyl, ethyl, and propyl esters). Any of the phosphate oxygens not alkylated can be OH or OM, where M represents a pharmaceutically acceptable cation. Furthermore, a hydroxyl of R¹ can be acylated with the carboxylic acid portion of an amino acid or of a dipeptide formed from two amino acids; such esters are particularly susceptible to in vivo hydrolysis by esterase activity. Accordingly, such esters can often serve as prodrugs that release the corresponding alcohol in vivo; and certain of such prodrugs are shown in Table 1 herein. These compounds may also possess intrinsic activity as effectors of TGFβ; accordingly, they are also useful as drugs themselves.

In the event that any of the substituents of formula (I) contain chiral centers or rotational isomers (atropisomers), as some, indeed, do, the compounds of formula (1) include each stereoisomeric form thereof, both as an isolated stereoisomer and as a component of a mixture of these stereoisomeric forms. Such mixtures of stereoisomers may be racemic or may be enriched in one enantiomer of a pair of enantiomers where a single chiral center is present. Where more than one stereoisomeric center is present, the invention includes mixtures wherein either, neither or both centers are enriched in one stereoisomeric form.

Synthesis of the Invention Compounds

A number of synthetic routes may be employed to produce the compounds of the invention. In general, they may be synthesized from conventional starting materials using reactions known in the art. Illustrative methods are provided below, and additional methods are described in published patent applications US 2004-0132159-A1 and US 2005/0004143-A1, which are incorporated by reference for their description of these synthetic methods.

Scheme 1 shows a general method for constructing pyrimidine rings having the substitution pattern required for compounds of the invention. First, an amidine is prepared; these can typically be made from the corresponding aryl nitriles as illustrated. The amidine is then allowed to react with a substituted malonaldehyde derivative to provide a 2-aryl substituted pyrimidinone. The group represented by X in Scheme 1 is typically alkyl, aryl, cycloalkyl, alkoxy, or dialkylamino.

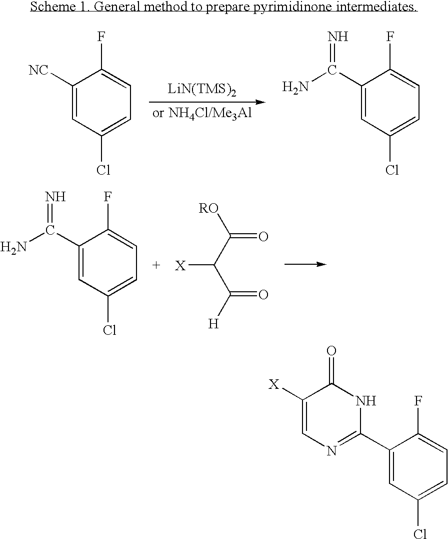

Scheme 2 illustrates a general strategy that was used to prepare many of the compounds of the invention, some of which are included in Table 1. The pyrimidinone ring is produced by cyclizing an amidine moiety as shown above, and the pyrimidinone is converted into a 3-halopyrimidine, typically with thionyl chloride/DMF or with POCl₃. The halo group on the pyrimidine ring is then displaced by a 3-substituted 4-aminopyridine to obtain a versatile intermediate having a carboxylate ester on the pyridine ring. This ester group is readily hydrolyzed to the free carboxylic acid as shown in Scheme I, and then can easily be converted into a wide variety of carboxamides of the invention having the A group of formula (I) linked to the pyridyl ring through the carbonyl carbon.

The malonaldehydes required for this reaction are typically prepared by formylation of the corresponding esters, using LDA and ethyl formate. Using these conditions, compounds can readily be prepared wherein X represents an alkoxy, alkyl, aryl, heteroaryl, or dialkylamine, for example.

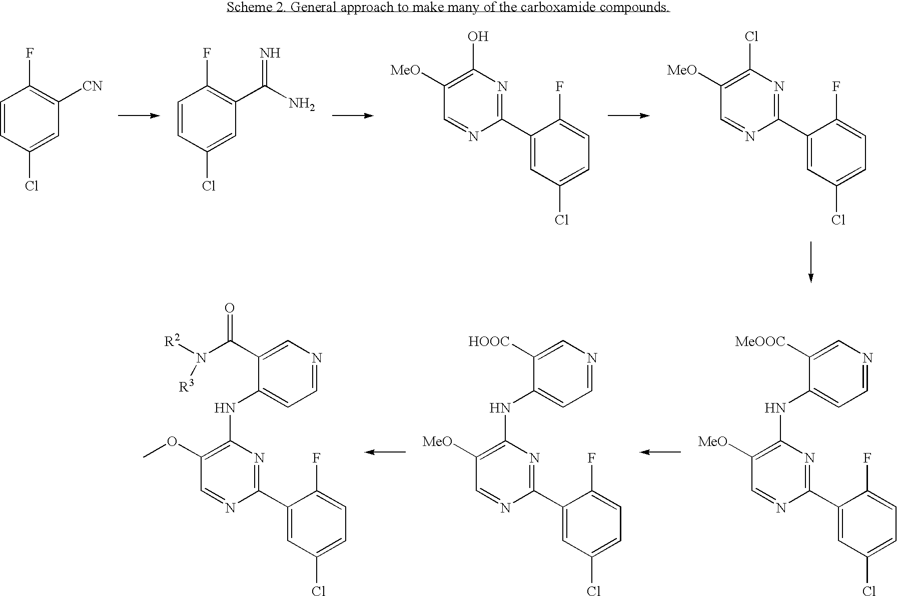

Reaction Schemes 3 and 4, shown below, provide routes to the pyrimidine nucleus that permit further substitution thereof. A malonate or cyanoacetate derivative is used to form the pyrimidinones in Schemes 3 and 4 rather than the malonaldehyde derivative used above. This provides pyrimidines having a substituent at position 6, corresponding to Z in formula (I).

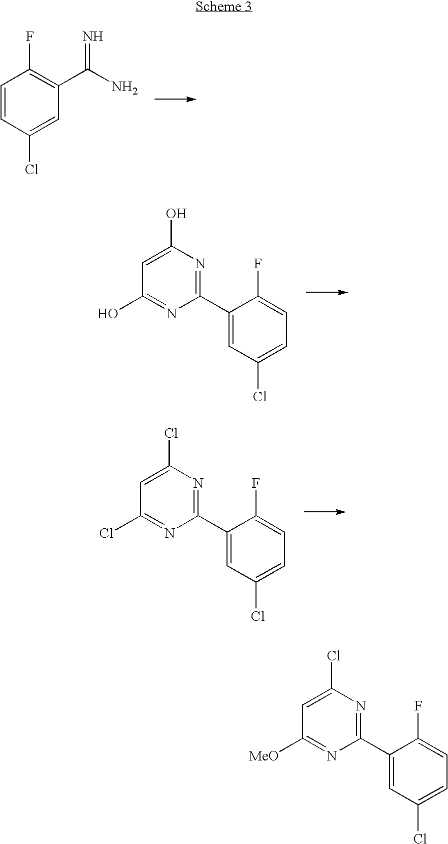

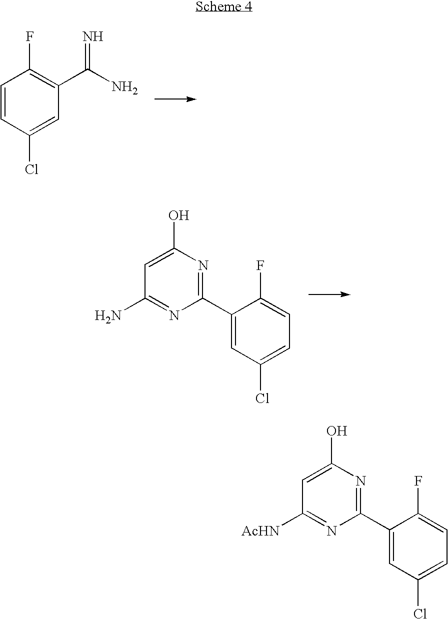

Scheme 5 below illustrates how the 3-carboxy-substituted-4-amino pyridines used in Scheme 2 above can be prepared via pyridine metalation chemistry. The metalation introduces a carboxylic acid or ester adjacent to the protected 4-aminopyridine. The 4-aminopyridine produced in this way can be coupled to an aryl pyrimidine as shown in Scheme 6, by cleaving the t-BOC from the amine substituent on the pyridine ring. The ester can then be converted into the desired carboxamide by hydrolysis followed by amide formation shown in Scheme 6. Alternatively, the ester can first be converted into a desired amide, and can then be attached to the halopyrimidine. The former approach is often used, however, so that the preparation of a single carboxylic acid compound permits a wide variety of carboxamide products to be made, each in a single step using well-known amide formation conditions.

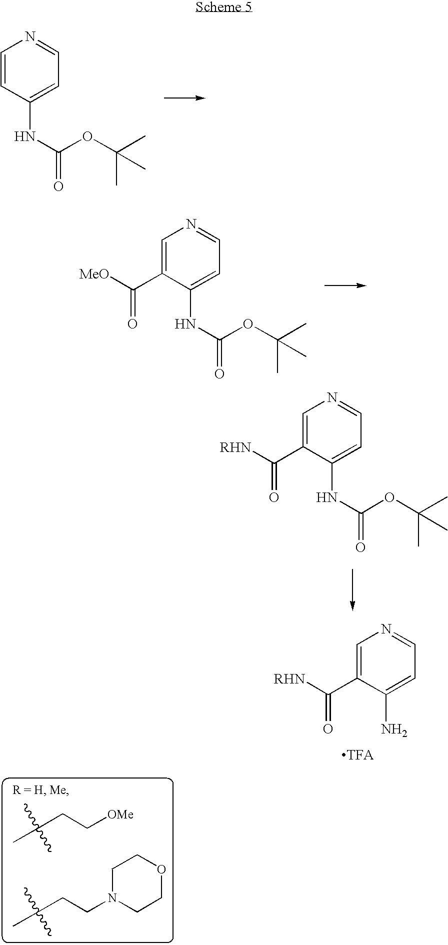

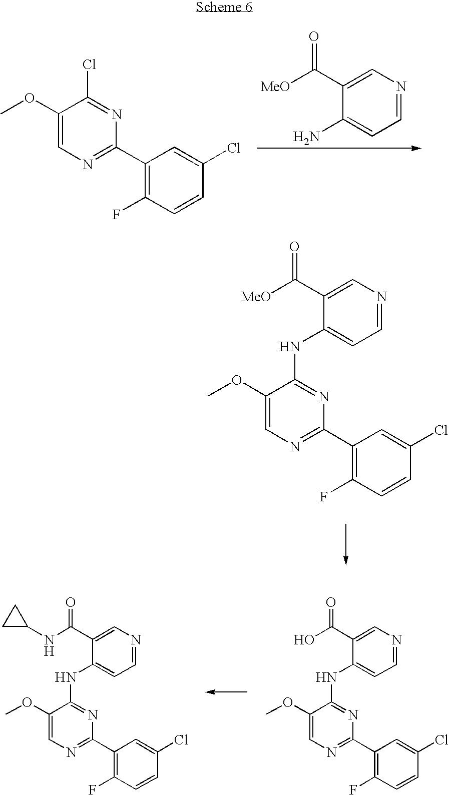

This scheme can be generally used to make 5-methoxy pyrimidine compounds of the invention, and was employed to synthesize many of the compounds in Table 1. Furthermore, other 5-alkoxy derivatives are available from this scheme, because the methoxy group can be cleaved using lithium iodide in hot DMF as is known in the art. The resulting hydroxypyrimidine can be O-alkylated or otherwise derivatized under conditions well known for the introduction of alkoxy, acyloxy, and similar substituents.

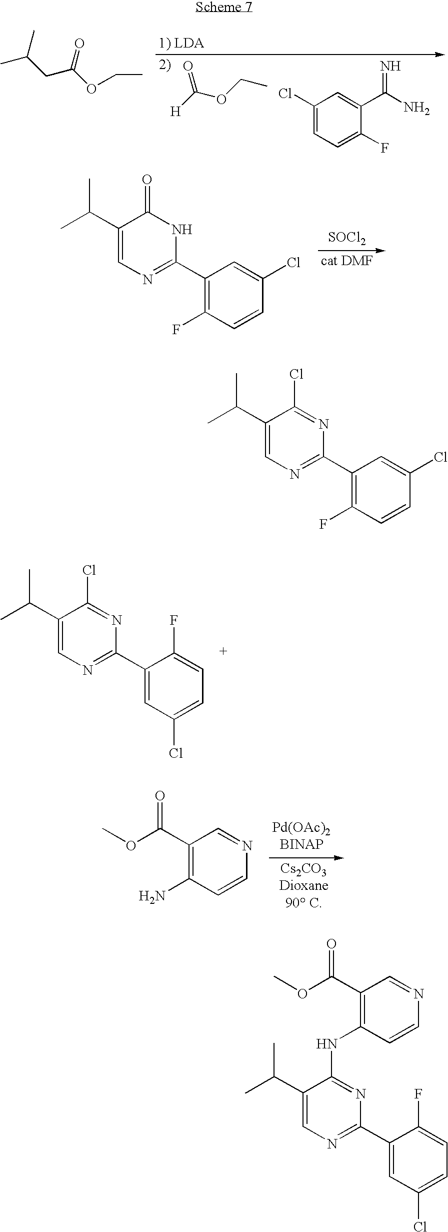

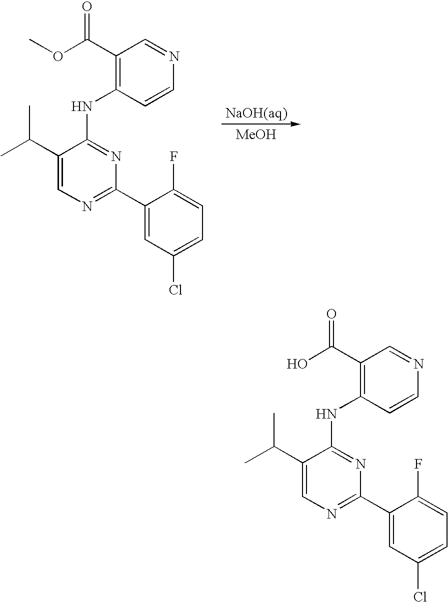

This scheme can be generally used to make isopropyl pyrimidines within the scope of the invention by coupling the carboxylic acid prepared in Scheme 7 with various amines. The use of a palladium catalyst to effect the coupling of the aminopyridine to the chloropyrimidine can be avoided by the use of a stronger base such as sodium hexamethyldisilazane as described in the Examples below (see Example 3).

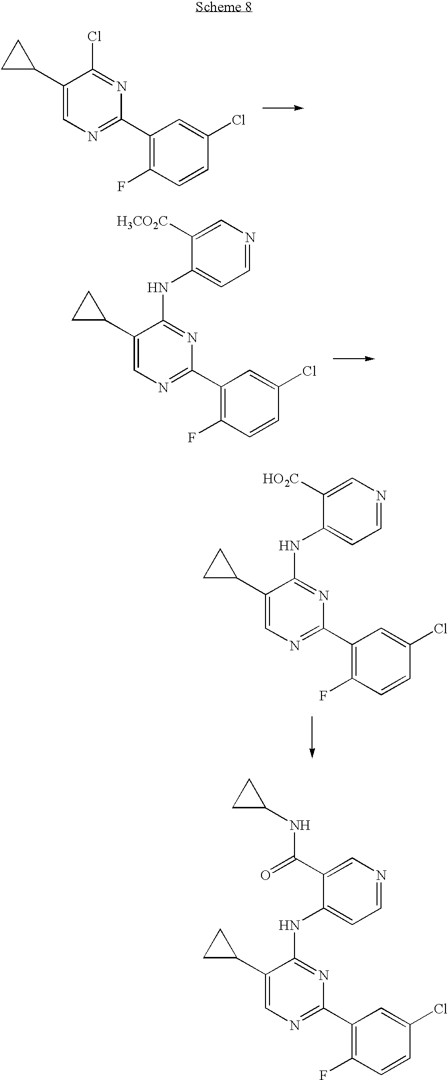

Scheme 8 depicts the preparation of a compound having a cyclopropyl group at position 5 of the pyrimidine ring. This method can be used to make 5-cyclopropyl pyrimidines having various carboxamide groups on the pyridine ring.

Scheme 9 depicts the corresponding synthesis of 5-cyclobutyl pyrimidine compounds, and shows the preparation of the methyl ester of cyclobutyl acetic acid from which the pyrimidine is constructed.

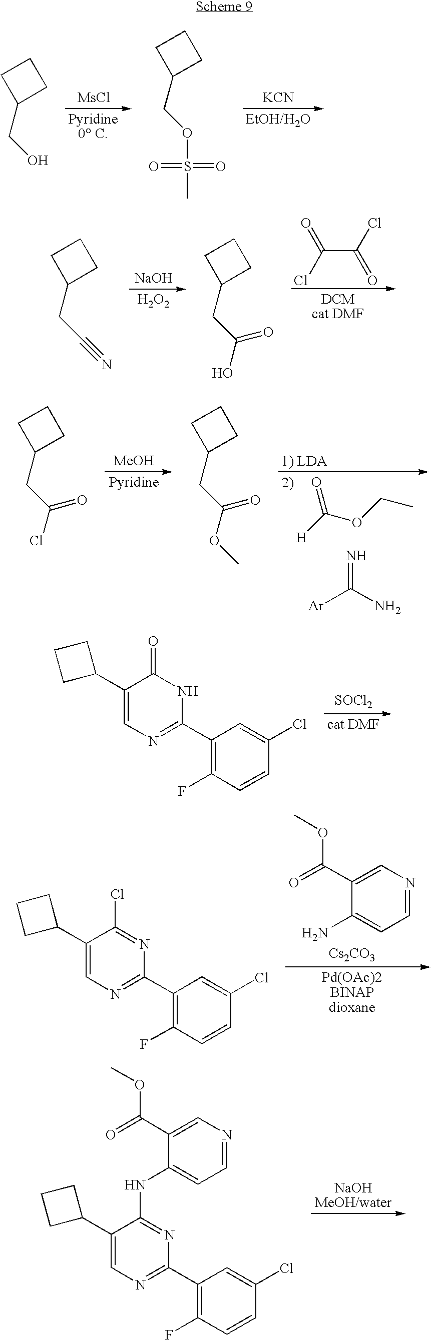

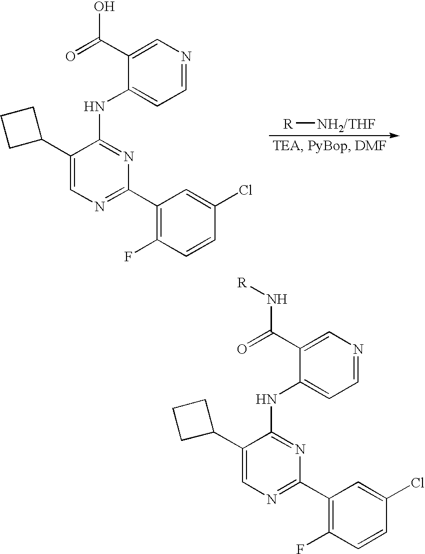

Scheme 10 shows the synthesis of 5-dimethylamino compounds of the invention using the same general approach. Cyclic amines can be introduced similarly.

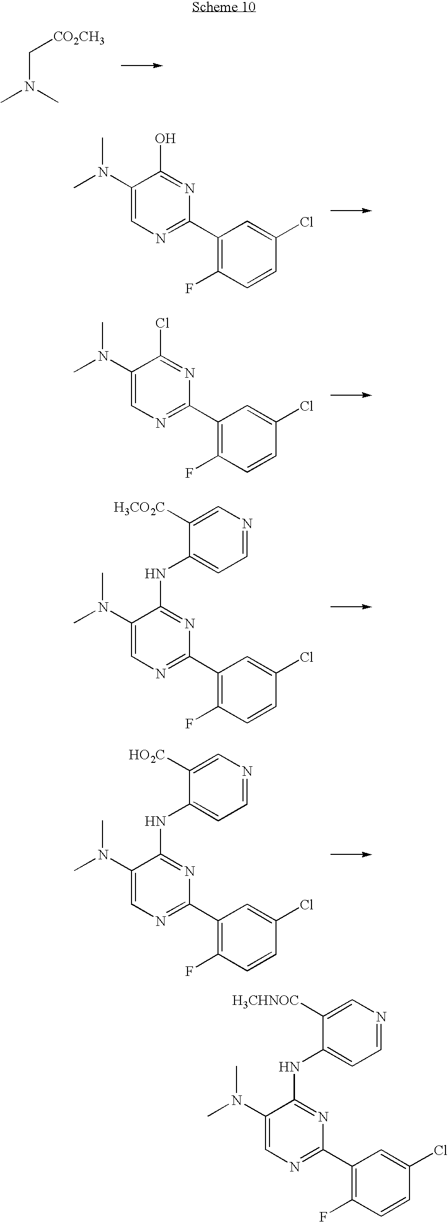

Scheme 11 can be generally used to make benzyloxy pyrimidines, including ones with substitution on the benzyl group, as well as to make other alkoxy substituted compounds. Like the methoxy compounds, these benzyloxy compounds can be used to make other 5-O-substituted compounds by removing the benzyl group using a catalytic hydrogenation, for example, followed by alkylation or acylation of the resultant hydroxypyrimidine.

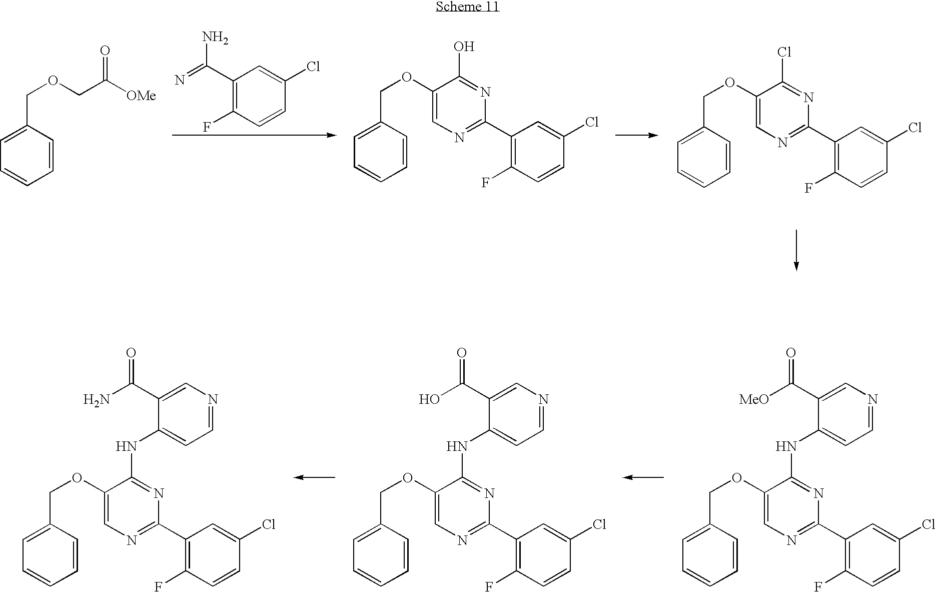

Scheme 12 illustrates use of the method described above for the preparation of compound of the invention where Y in formula (I) is a tert-butyl group.

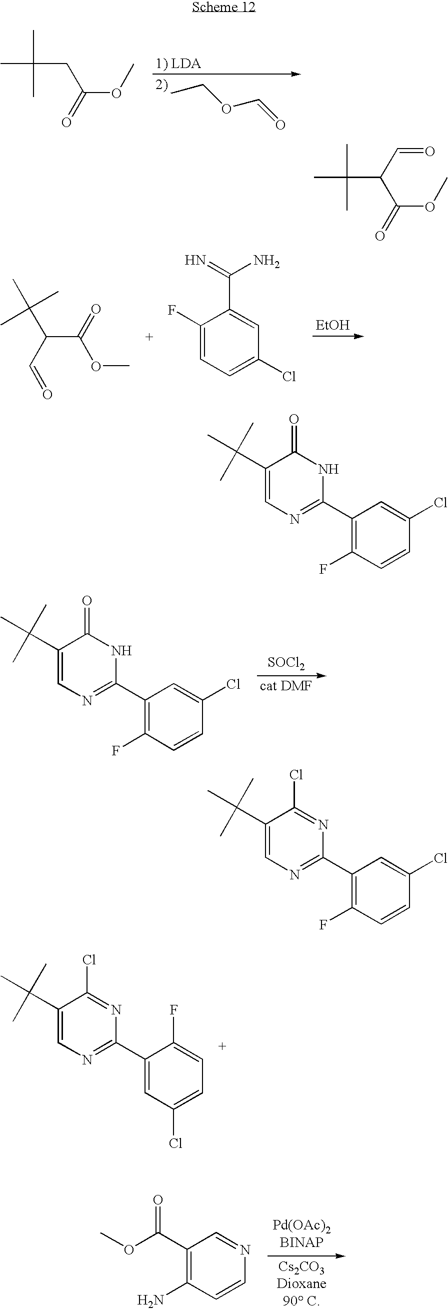

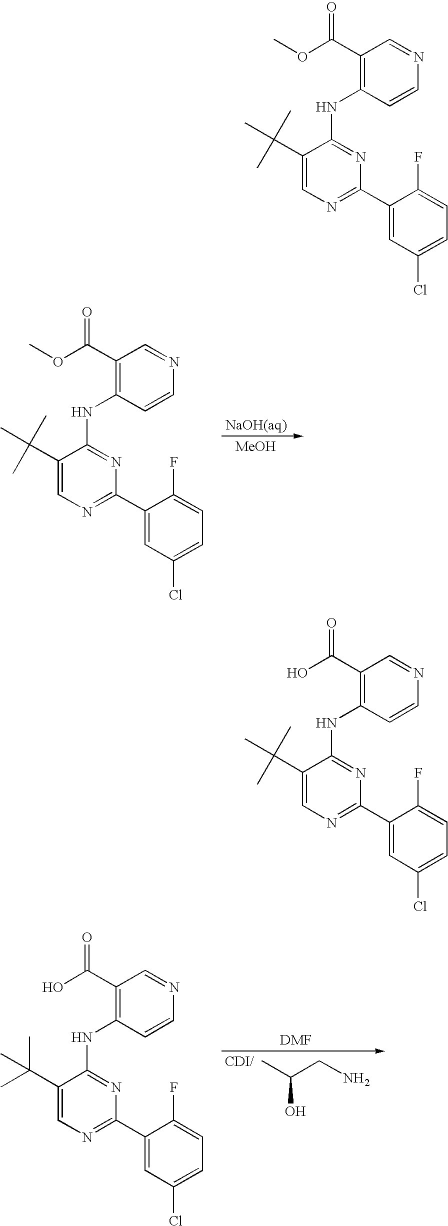

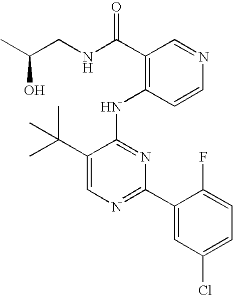

While this example shows the preparation of a compound where the group corresponding to R¹ in formula (1) has an undesired secondary hydroxyl, it can be used to introduce R¹ groups with a tertiary hydroxyl such as those described above, as well as many other variations of R¹ that are within the scope of formula (1) as described herein.

Where the pyridine N-oxides of compounds of formula (1) are desired, the pyridine compounds can be oxidized to N-oxides using commonly known oxidation reagents such as, for example, meta-chloroperoxy benzoic acid or peracetic acid.

Administration and Use

The compounds of the invention are useful in treating conditions associated with fibroproliferation. Thus, the compounds of formula (I) or their pharmaceutically acceptable salts or prodrug forms are used in the manufacture of a medicament for prophylactic or therapeutic treatment of mammals, including humans, in respect of conditions characterized by excessive activity of TGFβ.

TGFβ inhibition activity is useful in treating fibroproliferative diseases, treating collagen vascular disorders, treating eye diseases associated with a fibroproliferative condition, preventing excessive scarring, treating neurological conditions and other conditions that are targets for TGFβ inhibitors and in preventing excessive scarring that elicits and accompanies restenosis following coronary angioplasty, cardiac fibrosis occurring after infarction and progressive heart failure, and in hypertensive vasculopathy, and keloid formation or hypertrophic scars occurring during the healing of wounds including surgical wounds and traumatic lacerations.

Neurological conditions characterized by TGFβ production include CNS injury after traumatic and hypoxic insults, Alzheimer's disease, and Parkinson's disease.

Other conditions that are potential clinical targets for TGFβ inhibitors include myelofibrosis, tissue thickening resulting from radiation treatment, nasal polyposis, polyp surgery, liver cirrhosis, and osteoporosis.

Diseases benefited by TGFβ inhibition include cardiovascular diseases such as congestive heart failure, dilated cardiomyopathy, myocarditis, or vascular stenosis associated with atherosclerosis, angioplasty treatment, or surgical incisions or mechanical trauma; kidney diseases associated with fibrosis and/or sclerosis, including glomerulonephritis of all etiologies, diabetic nephropathy, and all causes of renal interstitial fibrosis, including hypertension, complications of drug exposure, such as cyclosporin, HIV-associated nephropathy, transplant nephropathy, chronic ureteral obstruction; hepatic diseases associated with excessive scarring and progressive sclerosis, including cirrhosis due to all etiologies, disorders of the biliary tree, and hepatic dysfunction attributable to infections such as hepatitis virus or parasites; syndromes associated with pulmonary fibrosis with consequential loss of gas exchange or ability to efficiently move air into and out of the lungs, including adult respiratory distress syndrome, idiopathic pulmonary fibrosis, or pulmonary fibrosis due to infectious or toxic agents such as smoke, chemicals, allergens, or autoimmune disease; all collagen vascular disorders of a chronic or persistent nature including progressive systemic sclerosis, polymyositis, scleroderma, dermatomyositis, fascists, or Raynaud's syndrome, or arthritic conditions such as rheumatoid arthritis; eye diseases associated with fibroproliferative states, including proliferative vitreoretinopathy of any etiology or fibrosis associated with ocular surgery such as retinal reattachment, cataract extraction, or drainage procedures of any kind; excessive or hypertrophic scar formation in the dermis occurring during wound healing resulting from trauma or surgical wounds; disorders of the gastrointestinal tract associated with chronic inflammation, such as Crohn's disease or ulcerative colitis or adhesion formation as a result of trauma or surgical wounds, polyposis or states post polyp surgery; chronic scarring of the peritoneum associated with endometriosis, ovarian disease, peritoneal dialysis, or surgical wounds; neurological conditions characterized by TGFβ production or enhanced sensitivity to TGFβ, including states post-traumatic or hypoxic injury, Alzheimer's disease, and Parkinson's disease; diseases of the joints involving scarring sufficient to impede mobility or produce pain, including states post-mechanical or surgical trauma, osteoarthritis and rheumatoid arthritis; and cancer.

The modulation of the immune and inflammation systems by TGFβ (Wahl, et al., Immunol. Today (1989) 10:258-61) includes stimulation of leukocyte recruitment, cytokine production, and lymphocyte effector function, and inhibition of T-cell subset proliferation, B-cell proliferation, antibody formation, and monocytic respiratory burst. TGFβ is a stimulator for the excess production of extracellular matrix proteins, including fibronectin and collagen. It also inhibits the production of enzymes that degrade these matrix proteins. The net effect is the accumulation of fibrous tissue which is the hallmark of fibroproliferative diseases.

TGFβ is active as a homodimer, but is synthesized and secreted from cells as an inactive latent complex of the mature homodimer and proregions, called latency associated protein (LAP). These proteins bind to each other through noncovalent interactions (Lyons and Moses, Eur. J. Biochem. (1990) 187:467). LAP is often disulfide-linked to separate gene products, called latent TGFβ binding proteins or LTBP's. These latent forms provide stability for the mature cytokine and a means for targeting it to the extracellular matrix and cell surfaces (Lawrence, Eur. Cytokine Network (1996) 7:363-74). Activation of the latent complex occurs after secretion from cells and is believed to result from the action of proteases, such as plasmin (Munger, et al., Kidney Intl. (1997) 51:1376-82), on LAP, thrombospondin-1 binding (Crawford, et al., Cell (1998) 93:1159-70), and binding to the integrin v6 (Munger, et al., Cell (1999) 319-28).

Other than αvβ there is a variety of cell surface proteins/receptors that transduce the signals initiated by binding of the active TGFβ ligand to its receptors. These include types I, II, III, IV, and V. Type IV is present only in the pituitary gland while the others are ubiquitous. The binding affinities among the three isoforms for the type I and II receptors differ such that these two receptors bind TGFβ1 and TGFβ3 more tightly than TGFβ2 (Massague, Cell (1992) 69:1067-70).

The type IV receptor or endoglin has a similar isoform binding profile in contrast to the type III receptor, betaglycan, which binds equally well to all three isoforms (Wang, et al., Cell (1991) 67:797-805; Lopez-Casillas, Cell (1991) 67:785-95). The type V receptor binds to IGFBP-3 and is thought to have an active kinase domain similar to the type I and II receptors. Cloning of the type I and type II receptors demonstrated the existence of cytoplasmic serine/threonine kinase domains (Wrana, et al., Cell (1992) 71:1003-14; Lin, et al., Cell (1992) 68:775-85; Ibid. 71:1069; Massague, Cell (1992) 69:1067-70). Initiation of the TGFβ signaling pathway results from the binding of the TGFβ ligand to the extracellular domain of the type II receptor (Massague, Ann. Rev. Biochem. (1998) 67:753-91). The bound receptor then recruits type I receptor into a multimeric membrane complex, whereupon the constitutively active type II receptor kinase phosphorylates and activates type I receptor kinase. The function of the type I receptor kinase is to phosphorylate a receptor-associated co-transcription factor, smad-2/3, thereby releasing it into the cytoplasm where it binds to smad-4. This smad complex translocates into the nucleus, associates with a DNA-binding cofactor, such as Fast-1, binds to enhancer regions of specific genes, and activates transcription. The expression of these genes leads to the synthesis of cell cycle regulators that control proliferative responses or extracellular matrix proteins that mediate outside-in cell signaling, cell adhesion, migration, and intercellular communication.

The manner of administration and formulation of the compounds useful in the invention and their related compounds will depend on the nature of the condition, the severity of the condition, the particular subject to be treated, and the judgment of the practitioner; formulation will depend on mode of administration. As the compounds of the invention are small molecules, they are conveniently administered by oral administration by compounding them with one or more suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of 5%-95% of the total formulation, but wide variation is permitted depending on the carrier. Suitable carriers include sucrose, pectin, magnesium stearate, lactose, peanut oil, olive oil, water, and the like.

The compounds useful in the invention may also be administered through suppositories or other transmucosal vehicles. Typically, such formulations will include excipients that facilitate the passage of the compound through the mucosa such as pharmaceutically acceptable detergents.

The compounds may also be administered topically, for topical conditions such as psoriasis, or in formulation intended to penetrate the skin. These include lotions, creams, ointments and the like which can be formulated by known methods.

The compounds may also be administered by injection, including intravenous, intramuscular, subcutaneous or intraperitoneal injection. Typical formulations for such use are liquid formulations in isotonic vehicles such as Hank's solution or Ringer's solution.

Alternative formulations include nasal sprays, liposomal formulations, slow-release formulations, and the like, as are known in the art.

Any suitable formulation may be used. A compendium of art-known formulations is found in Remington's Pharmaceutical Sciences, latest edition, Mack Publishing Company, Easton, Pa. Reference to this manual is routine in the art.

The dosages of the compounds of the invention will depend on a number of factors which will vary from patient to patient. However, it is believed that generally, the routine oral dosage will utilize 0.001-100 mg/kg total body weight, preferably from 0.01-50 mg/kg and more preferably about 0.01 mg/kg-10 mg/kg. Dosages will typically be administered at least once per day, but the dose regimen will vary, depending on the conditions being treated and the judgment of the practitioner.

It should be noted that the compounds of formula (I) can be administered as individual active ingredients, or as mixtures of several embodiments of this formula. The compounds of the invention may be used as single therapeutic agents or in combination with other therapeutic agents. Drugs that could be usefully combined with these compounds include natural or synthetic corticosteroids, particularly prednisone and its derivatives, monoclonal antibodies targeting cells of the immune system, antibodies or soluble receptors or receptor fusion proteins targeting immune or non-immune cytokines, and small molecule inhibitors of cell division, protein synthesis, or mRNA transcription or translation, or inhibitors of immune cell differentiation or activation.

As indicated above, although the compounds of the invention may be used in humans, they are also available for veterinary use in treating animal subjects.

### EXAMPLES

The following examples are intended to illustrate, but not to limit, the invention. Certain of the examples illustrate methods that are readily adapted to synthesis of compounds of formula (1), even though the specific example may not fit within formula (1) as described herein. As one of ordinary skill will appreciate, it is possible to combine various embodiments and synthesis methods described herein and to modify the starting materials by using well known or commercial alternatives to produce many variants that are not illustrated here: such combinations and variations are within the scope of the invention.

### Example 1

### Synthesis of [2-(3-chlorophenyl)-pyrimidin-4-yl]pyridin-4-yl amine

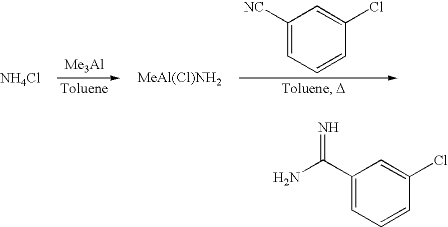

To a vigorously stirred, cooled (0° C.) suspension of (pestle-ground) ammonium chloride (1.17 g, 21.8 mmol) in dry toluene (7 mL) was added a solution of trimethylaluminum (10.9 mL, 2M solution in hexanes, 21.8 mmol) dropwise over 20 min. Effervescence occurred on addition. The mixture was stirred at r.t. for 15 min. To this solution was added a solution of 3-chlorobenzonitrile (1.0 g, 7.2 mmol) in dry toluene (5 mL) dropwise over 10 min. The solution was heated to 80° C. for 12 h then cooled and transferred slowly into a vigorously stirred slurry of silica gel (30 g) in chloroform (100 mL). The slurry was left stirred at r.t. for 10 min., then filtered. The filter cake was washed with methanol (3×100 mL) and the filtrate evaporated to a white solid that was dissolved in 10% aq. HCl (100 mL) and diethyl ether (50 mL). The solution was shaken and the organic layer discarded. The aqueous layer was basified to pH 14 with satd. aq. NaOH, and extracted with chloroform (3×100 mL). The organic extracts were dried over sodium sulfate and evaporated to a yellow oil that solidified (813 mg, 72%).

EIMS: 154 M+.

Alternatively, these amidine intermediates can be synthesized using lithium bis(trimethylsilyl)amide:

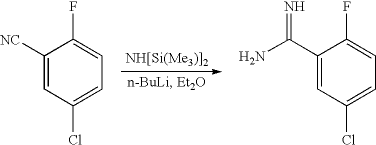

To a stirred 0° C. solution of 1,1,1,3,3,3-Hexamethyldisilazane (63 mL, 0.3 mol) in dry diethyl ether was added dropwise n-Butyl lithium (2M in hexanes, 150 mL, 0.3 mol). A white suspension formed, to which was added 2-Fluoro-5-chlorobenzonitrile (21.0 g, 0.14 mol) over 5 min. The resultant orange mixture was allowed to warm to r.t. and stirred for 2 h. The mixture was cooled to 0° C. and the reaction quenched by the addition of 3M HCl (aq.) (240 mL). The mixture was stirred for 0.5 h before water (600 mL) was added. The purple organic layer was discarded and the aqueous layer basified to pH 14 with satd. NaOH (aq.). The aqueous layer was extracted with CHCl₃ (5×100 mL) and the organic extracts dried over Na₂SO₄. Evaporation yielded the desired product as a yellow solid (16.2 g, 73% yield).

Compounds having no substituent at the 5-position of the pyrimidine (Y═H) can be made from the amidines by using a propiolic acid ester in place of the malonaldehyde or malonate derivatives that are typically used in Scheme 2, for example.

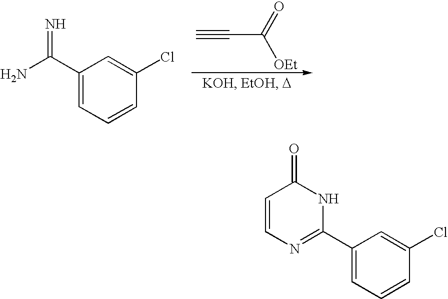

To a solution of 3-Chlorobenzamidine (1 g, 6.47 mmol) in dry ethanol (20 mL) was added ethyl propiolate (983 mL, 9.70 mmol) dropwise over 1 min. The solution was heated to 60° C. and a solution of potassium hydroxide (640 mg, 9.70 mmol) in dry ethanol (15 mL) was added dropwise over 1 h. Once added, the mixture was heated at 80° C. for 24 h, then cooled and evaporated. The residue was dissolved in water and the solution acidified with 10% aq. HCl to pH 4, whereupon a white precipitate formed, which was filtered and dried in vacuo (742 mg, 56%).

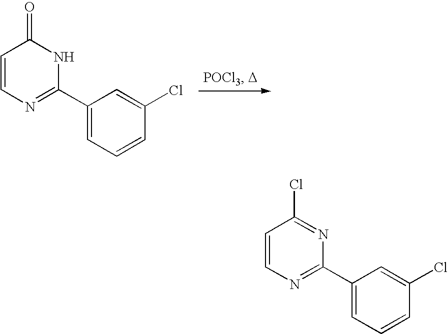

A suspension of the crude 2-(3-Chlorophenyl)-pyrimidin-4-one (197 mg, 0.9 mmol) in phosphorus oxychloride (5 mL) was heated to reflux for 0.5 h, then cooled and evaporated. The residue was purified by chromatography (eluting with CHCl₃) to yield the desired product as a white solid (191 mg, 89% yield). EIMS: 225 M+.

This intermediate can be used to make the carboxamide compounds of the invention by methods described herein: the 4-chloro substituent on the pyrimidine can be displaced by aminopyridines as described below.

### Example 2

### Preparation of a 2-methoxy malonaldehyde

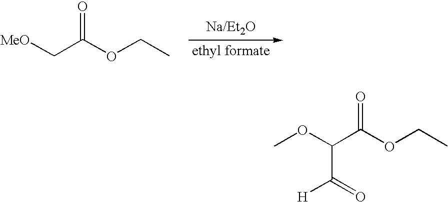

To a solution of ethyl formate (4.2 g, 56.7 mmol) in ether (80 ml) at 0° C. under N₂ was added small pieces of metal Na followed by dropwise addition of ethyl methoxy acetate (6.69 g, 56.70 mmol). The reaction was stirred for 30 min at 0° C. and was allowed to warm to ambient temperature. After 4 h at room temperature, the reaction was worked up and the product was used without further purification. The product was kept refrigerated as a stock solution.

### Example 3

### Preparation of 5-Isopropyl Pyrimidine Compounds

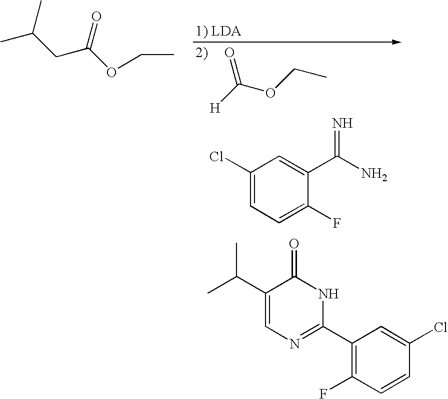

To a solution of disopropylamine (15.4 ml, 110 mmole) in 30 ml tetrahydofuran (anh.) at −20° C. was added dropwise, n-butyllithium (2.5M hexane, 48 ml, 120 mmol). The solution was stirred at 0° C. for 40 min. The mixture was then cooled to −78° C. and ethyl isovalerate (13.0 g, 100 mmol) was added dropwise, the reaction mixture was stirred at −78° C. for 30 min. Ethyl formate (7.41 g, 100 mmol) was then added and the reaction mixture was warmed to room temperature with stirring for 1 hour. 5-chloro-2-fluorobenzamidine (17.0 g, 100 mmol) was dissolved in tetrahydrofuran (40 ml) and added to the reaction mixture over 10 min, followed by refluxing for 18 hr. Removed solvent under vacuum and residue was suspended in chloroform (150 ml) and water (150 ml). The basic aqueous phase was separated and filtered to remove some precipitate. The filtrate was acidified with glacial acetic acid to pH 5 and extracted with ethyl acetate (2×250 ml), washed combined extracts with saturated sodium chloride, dried over sodium sulfate (anh.) and removed the solvent to give 3.43 g product.

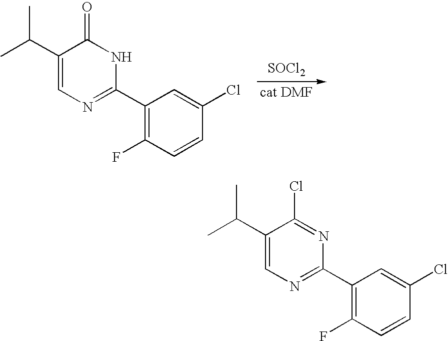

2-(5-chloro-2-fluorophenyl)-5-isopropylprimidine-4-one (3.43 g, 12.9 mmol) was suspended in thionyl chloride (15 ml, 205 mmol) and 3 drops DMF were added. The mixture was heated to 80° C. for 30 min, then excess thionyl chloride was removed under vacuum. The residue was treated with ice (50 ml) and chloroform (50 ml). Extracted product into a chloroform layer. Washed chloroform with 10% sodium carbonate (cold) and dried the chloroform layer over sodium sulfate (anh.). Solvent was then removed to give 3.32 g product.

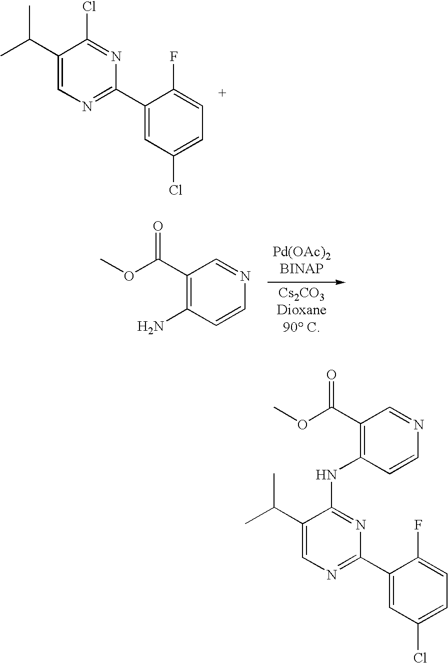

BINAP (233 mg, 0.375 mmole) and palladium(II)acetate (56.1 mg, 0.25 mmole were combined in 8 ml dioxane (anh) and heated for 5 min, followed by addition of 2-(5-chloro-2-fluorophenyl)-4-chloro-5-isoprpylpyrimidine (1.42 g, 5 mmole), methyl 4-amino-3-pyridinecarboxylate (912 mg, 6 mmole) and cesium carbonate (2.28 g, 7.0 mmole). The mixture was heated to 90° C. overnight. Removed dioxane under vacuum, the solid residue was triturated with ethyl acetate (20 ml) and filtered to give 767 mg product which contains cesium carbonate and was used directly in the next step without further purification.

Alternatively, the coupling can be achieved without using the palladium catalyst, if a stronger base is employed. This alternative is illustrated by the following example:

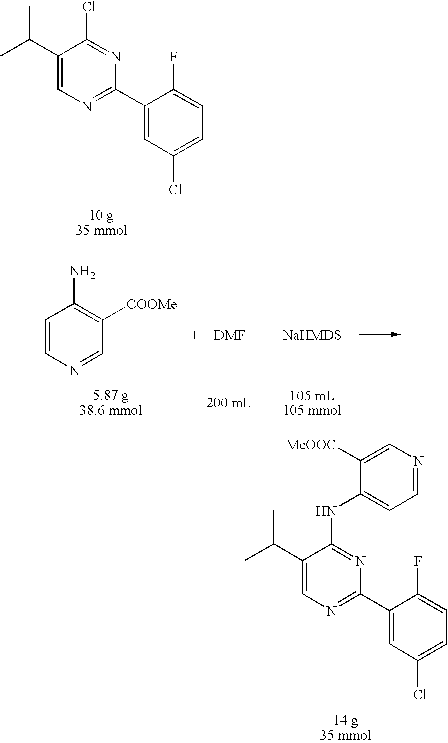

The chloropyrimidine (10 g, 35 mmol) and 4-amino-3-ester-pyridine (5.87 g, 38.6 mmol) were placed in an oven-dried flask (1 L), which was evacuated and flushed with nitrogen three times. Under nitrogen, anhydrous DMF (200 mL) was cannulated into the flask. Both materials were dissolved before the temperature was lowered to 0° C. Sodium hexamethyldisilazane (1M, 105 mL) in THF was then cannulated quickly into the solution. The mixture was stirred between 0° C. and 15° C. for 4 hrs. Saturated NH₄Cl solution (100 mL) was then added and the solvent was evaporated under vacuum. Saturated NH₄Cl solution (500 mL) and CH₂Cl₂ (500 mL) were then added to the crude mixture. After separation, the aqueous layer was further extracted by CH₂Cl₂ (500 mL×3). Combined organic layers were dried (Na₂SO₄), filtered and evaporated. The dark brown crude solid was triturated with 120 mL EtOAc to give light brown solid (6.66 g, 48%) as the pure desired product.

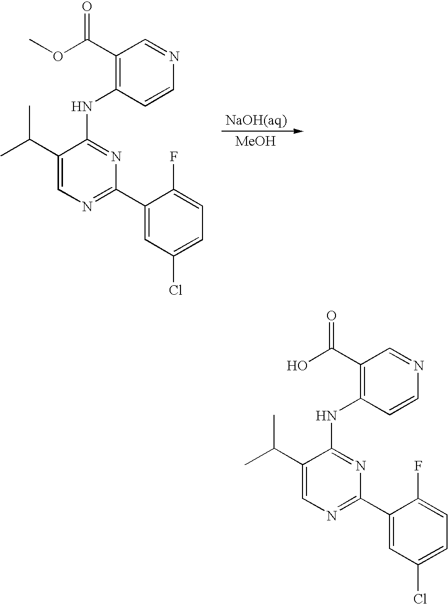

The above ester (630 mg, 1.57 mmole) was suspended in 10 ml methanol and treated with 4 ml 2.0M NaOH (aq). The mixture was refluxed for 30 min, then cooled and concentrated under vacuum to remove methanol. The aqueous solution was acidified with 6M HCl (pH 5), and filtered to obtain product; the yield was 180 mg.

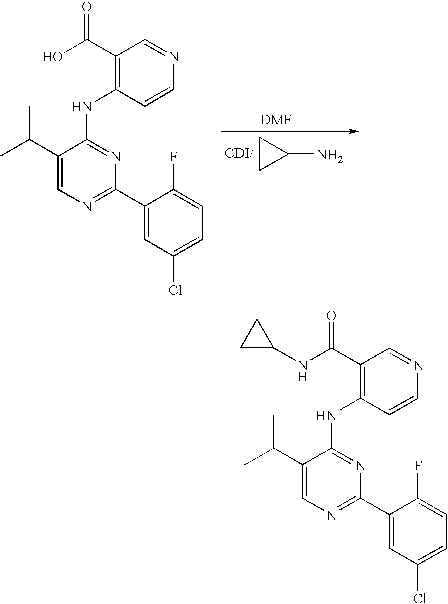

The acid (193 mg, 0.5 mmole) was suspended in DMF (anh., 6 ml) and treated with carbonyl diimidazole (162 mg, 1.0 mmole) and heated to 60° C. for 2 hours. Cyclopropylamine (114 mg, 2.0 mmole) was added and the solution was stirred overnight at room temperature. The mixture was filtered, and the filtrate was subjected to HPLC purification. Yield: 34 mg.

### Example 4

### Preparation of 5-Methoxy Pyrimidine Compounds

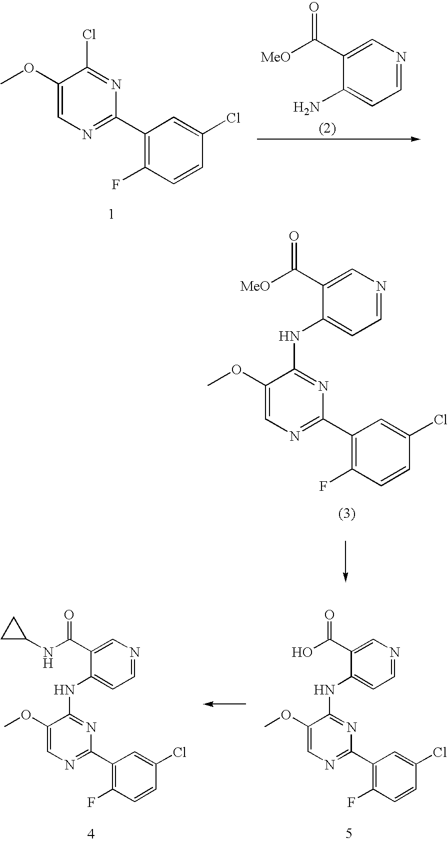

Preparation of 3:

The imino chloro compound 1 (5 g, 18.3 mmol, 1 eq), Pd₂(dba)₃ (670 mg, 0.7 mmol, 0.04 eq) and BINAP (684 mg, 1.1 mmol, 0.06 eq) were suspended in dioxane (280 mL) under inert atmosphere. A solution/suspension of the amine 2 (3.07 g, 20.2 mmol, 1.1 eq) in dioxane (90 mL) was added at a moderate speed, followed by Cs₂CO3 (11.9 g, 36.5 mmol, 2 eq). The mixture was then heated to 95° C. under nitrogen for 18 hours. The warm reaction mixture was then filtered through Celite® and the Celite® pad was washed with ethyl acetate (100 mL). The filtrate was then concentrated in vacuo to approx 100 mL in volume (not to dryness). The suspension was filtered and the solid washed with ethyl acetate and dried in vacuo. Product 3 was obtained as a cream colored solid 4.92 g, 69% yield: pure.

Preparation of 4:

A suspension of the Ester 3 (1.6 g, 4.1 mmol), NaOH (1.5-1.8 eq, 0.3 g, 7.5 mmol), water (5 mL) and dioxane (50 mL) was heated to 65° C. for 0.5 hour. The reaction was cooled to room temperature and 1M HCl solution was added until a pH 4 was obtained. The suspension was filtered and washed with water. The product 4 was dried in vacuo at 40° C. overnight., 1.1 g, 71% yield (cream solid)

Preparation of 5

A suspension of the acid 4 (1 g, 2.67 mmol) and CDI (0.865 g, 5.33 mmol, 2.0 eq) in dry DMF (20 mL) was heated at 75° C. for 0.5-2 hrs under N₂. The reaction was cooled to room temperature and cyclopropylamine (0.3 mL, 4.1 mmol, 1.5 eq) and triethylamine (0.4 mL, 2.67 mmol) were added. The reaction was stirred for 18 hours. The reaction mixture was then filtered and the solid washed with ethyl acetate. The pure product was obtained as a white solid, 0.71 g, 65% yield.

### Example 5

### Preparation of Compounds with a 5-Cyclopropyl Pyrimidine

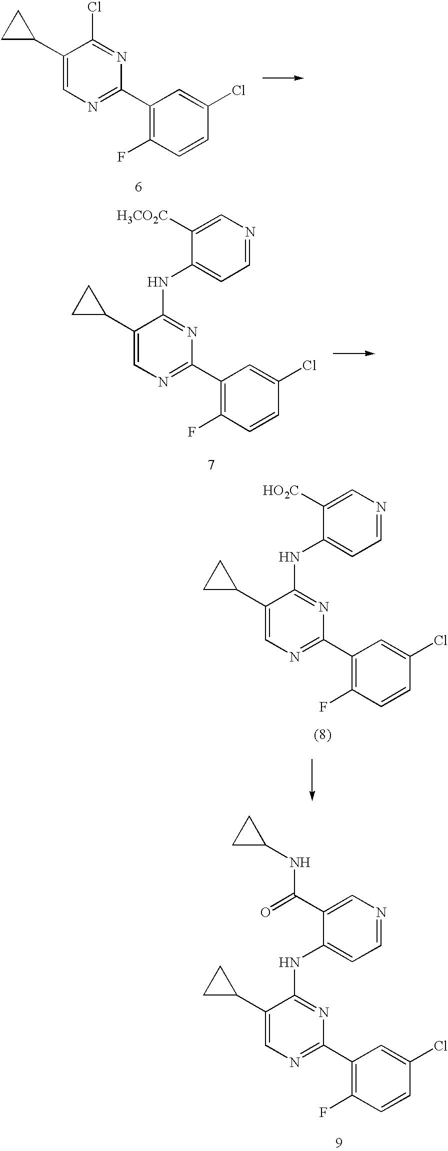

Preparation of 7:

To 1.42 g (5.0 mmol) of (6), was added 2.2 g (7.0 mmol) cesium carbonate, 0.056 g (0.25 mmol) Pd(OAc)₂, 0.233 g (0.44 mmol) BINAP, and 0.912 g (6.0 mmol) of 4-amino-3-methylester pyridine. 10 ml of anhydrous 1-4-dioxane was added and the mixture was heated to 90° C. overnight. Dioxane was removed by reduced pressure and material was washed with ethylacetate.

Preparation of 8:

To 0.35 g (1.24 mmol) of (7) was added 8 ml of methanol and 3 ml of a 1M NaOH solution. Mixture was heated to 70° C. for 2 hrs, cooled then acidified to pH5 using 1M HCl. Product was collected by vacuum filtration, washed with a small amount of water and dried in vacuum oven.

Preparation of 9:

To 0.223 g (0.589 mmol) of (8), was added 0.19 g (0.18 mmol) of N,N′-Carbonyldiimidazole. The mixture was treated with 4 ml of anhydrous DMF and heated to 70° C. for 2 hrs. Reaction was cooled to room temperature and 0.168 g (2.9 mmol) of cyclopropylamine was added and the reaction stirred at room temperature overnight. Reaction was then filtered and purified by prep HPLC.

### Example 6

### Preparation of a 5-Cyclobutyl Pyrimidine Compound

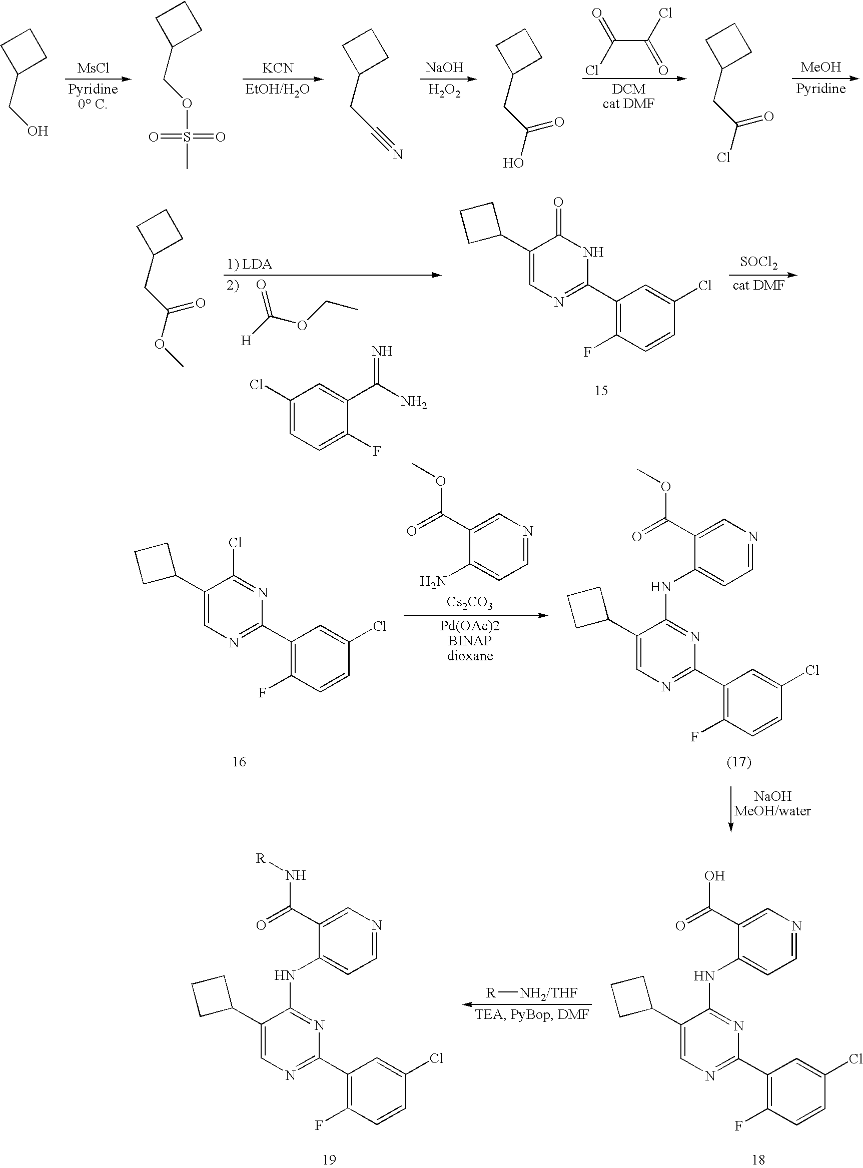

Preparation of Methyl Cyclobutylacetate:

A mixture of cyclobutylmethanol (25 g, 0.290 mole) and methanesulfonyl chloride (33.25 g, 0.290 mole) was stirred at 0° C. while pyridine was added drop wise over 2.5 hours. Reaction mixture was kept at 0° C. overnight, then combined with 150 ml ice cold 10% HCl. The mixture was extracted with diethyl ether (3×125 ml). Combined extracts were washed with water (2×20 ml) followed by saturated sodium bicarbonate (30 ml). Dried extract over anhydrous sodium sulfate and solvent removed under reduced pressure to give 35.58 g product.

Cyclobutymethylmesylate (35.38 g 0.215 mole) was dissolved in 250 ml 80% ethanol/water and treated with potassium cyanide (25.25 g, 0.388, 1.8 eq) and the reaction mixture was refluxed overnight. Poured reaction mixture into 200 ml water and extracted with diethyl ether (2×100 ml), then washed with saturated sodium chloride (˜50 ml). Dried ether over sodium sulfate (anh.). The dark brown solution was passed over Florisil® (˜10 cm I.D.×15 cm) twice to remove brown color. Removal of solvent gave crude product, which was purified further by vacuum distillation to give 9.5 g product.

An ice cooled bath of sodium hydroxide (40 g) in 50 ml water was stirred while a 30% hydrogen peroxide solution (50 ml) was added slowly maintaining cool temperature. Cyclobutylacetonitrile (9.5 g, 0.10 mole) was added slowly, and the solution was stirred 30 min then heated to reflux for 2 days. Cooled reaction mixture, extracted with 50 ml chloroform to remove unreacted nitrile. Acidified aqueous layer with conc. HCl to pH 2, extracted cooled mixture with chloroform (3×150 ml). Dried chloroform extract over magnesium sulfate (anh.). Evaporated solvent to give 8.63 g product.

Cyclobutylacetic acid (8.63 g, 75.6 mmole) was dissolved in dichloromethane containing 2 drops dimethylformamide, and oxalyl chloride (45 ml, 2M in dichloromethane) was added drop wise over 30 min at room temperature. The reaction mixture was stirred at room temperature for 3 hours, and then solvent removed to give 8.6 g product.

Cyclobutyl acetyl chloride (8.6 g, 64.8 mmole) was added dropwise to a stirred solution of pyridine (10.48 ml, 129.6 mmole) in methanol (105 ml). The solution was stirred overnight at room temperature. Most of the excess methanol was removed under vacuum. Solution was poured onto 150 ml water, extracted with diethyl ether (3×125 ml). Combined extracts were washed with 25 ml 10% HCl, water (25 ml) and saturated sodium bicarbonate (25 ml), water (25 ml), saturated sodium chloride (25 ml). Ether was dried over anhydrous sodium sulfate and solvent removed to give 5.90 g methyl cyclobutylacetate.

Preparation of 15:

To a solution of diisopropylamine (7.15 ml, 50.63 mmol) in 20 ml anhydrous tetrahydrofuran at −20° C., was added n-butyl lithium (2.5M hexanes, 22 ml, 55.23 mmol) drop wise. The solution was stirred at 0° C. for 40 min, and cooled reaction to −78° C. Methyl cyclobutyl acetate (5.9 g, 46.03 mmol) was added dropwise, and the reaction mixture was stirred at −78° C. for 30 min. Ethyl formate (3.71 ml, 46.03 mmol) was added and reaction mixture was warmed to −10° C. for 1 hour, then room temp 1 hour. 5-chloro-2-fluorobenzamidine (7.94 g, 46.03 mmol) was dissolved in 20 ml tetrahydrofuran and added to the reaction mixture dropwise over 10 min. The mixture was then refluxed overnight. Removed most of the tetrahydrofuran under vacuum, and residue was taken up in 200 ml water. Washed aqueous solution with diethyl ether (2×75 ml) which removed dark color. Aqueous phase was acidified with glacial acetic acid to pH 5. Product precipitated from solution. Filtered solid, washed with water and vacuum dried to give 3.77 g product. (29% yield).

Preparation of 16:

2-(5-chloro-2-fluoro)-5-cyclobutylpyrimidine-4-one (3.75 g, 13.5 mmole) was suspended in thionyl chloride (15 ml, 205 mmole), added 2 drops dimethylformamide and heated mixture to 80° C. for 30 min. Starting material was completed dissolved at this time. Removed excess thionyl choride under vacuum and residue was poured onto ice water and extracted with chloroform. The chloroform layer was washed with 10% sodium carbonate, and dried over anhydrous. Filtration and solvent removal give 3.98 g product. (99%).

Preparation of 17:

2-(5-chloro-2-fluoro)-4-chloro-5-cyclobutylpyrimidine (1.48 g, 5 mmol), cesium carbonate (2.28 g, 7 mmol), palladium(II) acetate (56.1 mg, 0.25 mmol), BINAP (233 mg, 0.375 mmol) and methyl 4-aminopyridine-3-carboxylate (912 mg, 6 mmol) were combined in dioxane and heated to 80° C. overnight. Removed solvent under vacuum, triturated residue with ethyl acetate, filtered solid, washed with ethyl acetate to give 4.20 g solid, estimated to contain 1.92 g product, along with remaining cesium carbonate. This material was used directly without further purification.

Preparation of 18:

The above crude material (4.20 g, estimated to contain 1.92 g starting material +cesium carbonate) was suspended in methanol 10 ml, and 10 ml 1M sodium hydroxide was added. Refluxed the solution for 1 hour, then cooled mixture, removed methanol under vacuum, acidified aqueous solution to pH 4 with 1M HCl, filtered solid washing with water to give 1.30 g product after vacuum oven drying.

Preparation of 19:

Compound 18 (130 mg, 0.326 mmole) was suspended in dimethylformamide (8 ml). To this was added Pybop (254 mg, 0.489 mmole), triethylamine (49 microliters, 0.359 mmole) and 2M methyl amine/THF (815 microliters, 1.63 mmole) and the reaction was stirred at room temperature for 3 hours. The reaction mixture was filtered through 0.45 micron filter and subjected to HPLC purification to give 61 mg product.

### Example 7

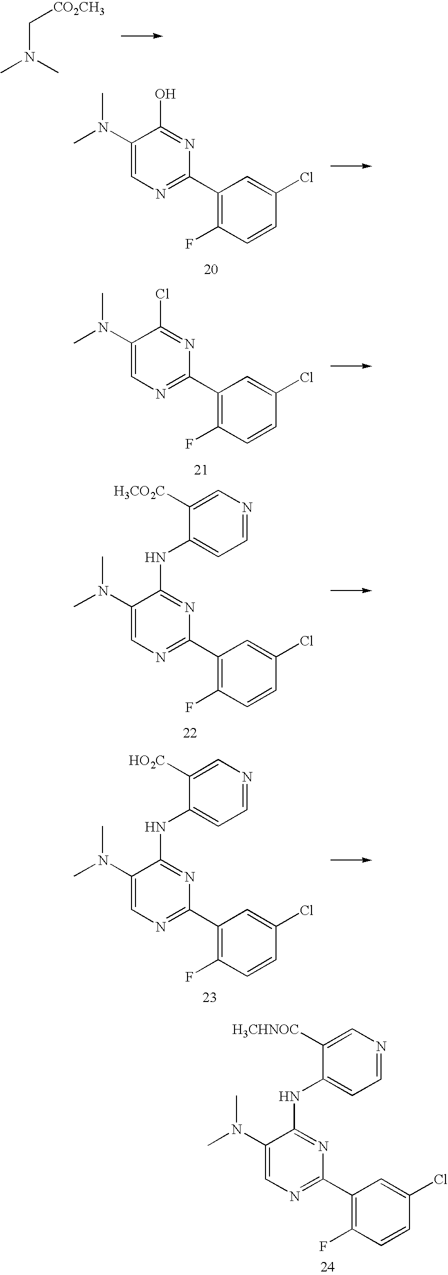

Preparation of 20:

Solid sodium metal pieces (2.11 g, 92.0 mmol) were washed with hexane and crushed into smaller pieces. Hexane was removed and the sodium pieces were added to a stirred solution at 0° C. of N,N-dimethylglycine methyl ester, (10.78 g, 92.0 mmol in anhydrous ether (80 ml)). Ethyl formate (7.4 ml, 92.0 mmol) was added dropwise to this solution and the reaction was stirred at room temperature for 3 hours. The reaction solution turned a creamy yellow consistency. To this mixture, 5-chloro-2-fluorobenzamidine, (15.9 g, 92.0 mmol) dissolved in 100 ml of 200 proof ethanol was syringed into the reaction flask and the mixture was refluxed gently overnight. Solvent was then removed under reduced pressure and slurry was taken up into chloroform and extracted with water. The aqueous layer was adjusted to pH 7 and extracted with chloroform. Combined organic solvent was dried using magnesium sulfate and concentrated. Crude product was then washed with 20% ethyl acetate/Hexane. Yield is 4.3 g, 17.5%.

Preparation of 21:

2-(5-chloro-2-fluorobenzyl)-5-cyclopropyl-pyrimidone, (0.46 g, 1.61 mmol) was treated with (2 ml, 15.7 mmol) of phosphorus oxychloride and refluxed for 2 hrs. Solvent was removed under reduced pressure and product was extracted into chloroform and washed with a saturated solution of sodium hydrogen carbonate cooled with ice. Organic solvent was dried using magnesium sulfate and concentrated. Reaction produced 0.43 g of product, 95% yield.

Preparation of 22:

Imino chloride (21), (0.43 g, 1.5 mmol) was dissolved in 5 ml of anhydrous 1,4-dioxane. To this (0.29 g, 1.9 mmol) of 5, (0.018 g, 0.080 mmol) of palladium acetate, (0.075 g, 0.121 mmol) of BINAP, and (0.786 g, 2.41 mmol) of cesium carbonate were added at once. The reaction was refluxed for 3 hours, cooled and the dioxane was evaporated off. Crude product was washed with ethyl acetate. The crude product was mixed with cesium carbonate. No yield was taken.

Preparation of 23:

To (22) was added 15 ml of methanol and 3 ml of a 1M NaOH solution. Mixture was heated to 70° C. for 2 hrs, cooled then acidified to pH4 using 1M HCl. Product was collected by vacuum filtration, washed with a small amount of water and dried in vacuum oven. Received 0.064 g, 10.3% collective yield from imino chloride (21).

Preparation of 24:

To (0.064 g, 0.166 mmol) of (23), was added (0.054 g, 0.330 mmol) of N,N′-Carbonyldiimidazole. The mixture was treated with 5 ml of anhydrous DMF and heated to 70° C. for 2 hrs. Reaction was cooled to room temperature and 0.249 ml (0.498 mmol) of methylamine was added and the reaction stirred at room temperature overnight. Reaction was then filtered and purified by prep HPLC. Received 0.0152 g of material, 22.7% yield.

### Example 8

### Preparation of a 5-Benzyloxy Pyrimidine Compound

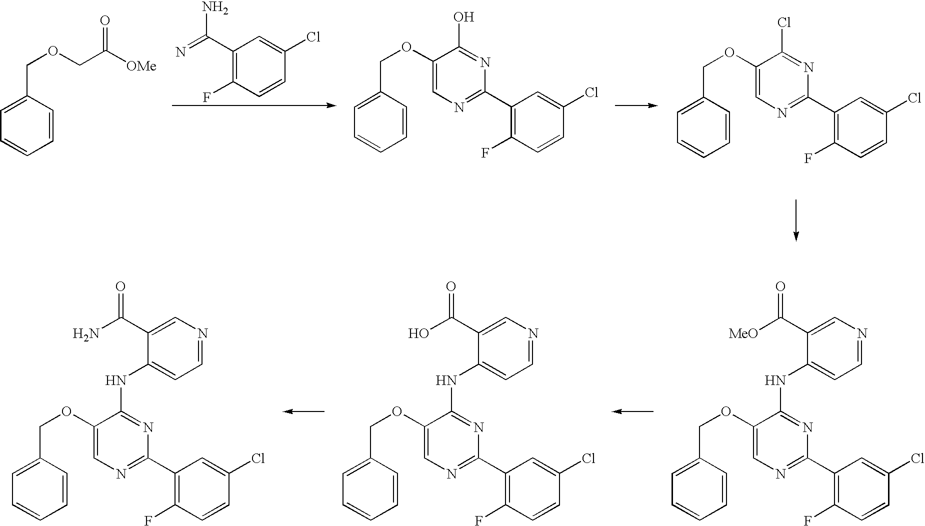

The 5-benzyloxy analogs were synthesized using the same conditions as those for the 5-methoxy analogs, but using methyl-benzyloxyacetate 31 as the starting material.

### Example 9

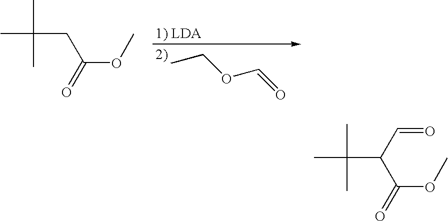

To a solution of diisopropylamine (20.58 g, 204 mmole) in 60 ml tetrahydrofuran (anh.) at −20° C. was added dropwise, n-butyllithium (2.5M hexane, 88 ml, 222 mmol). The solution was stirred at 0° C. for 40 min. The mixture was then cooled to −78° C. and methyl t-butyl acetate (24.1 g, 185 mmol) was added dropwise, the reaction mixture was stirred at −78° C. for 30 min. Ethyl formate (13.70 g, 185 mmol) was then added and the reaction mixture was warmed to room temperature with stirring for 18 hours. The reaction mixture was poured into 300 ml ice water. The organic layer was extracted with 1M sodium hydroxide (2×40 ml) and added to the aqueous layer. The aqueous layer was acidified with 40% sulfuric acid to pH 5.0 with cooling. The solution was extracted with diethyl ether (5×40 ml), combined ether extract washed with saturated sodium chloride, dried over sodium sulfate (anh.) and solvent removed to give product as a liquid (11.4 g, 39% yield). This material was used without further purification.

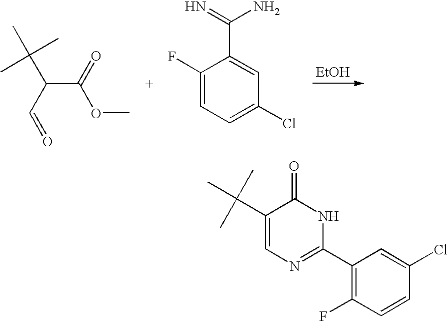

5-chloro-2-fluorobenzamidine (7.39 g, 42.8 mmole) and methyl 1-formyl-t-butyl acetate (6.78 g, 42.8 mmole) were dissolved in ethanol (75 ml) and heated to reflux for 2 hours. Removed ethanol by rotary evaporation; the residue was taken up in chloroform (300 ml), and was extracted with 1M sodium hydroxide (4×40 ml). Combined aqueous extract was acidified with 1M hydrochloric acid. Product was extracted with ethyl acetate (3×100 ml), the combined extract was dried over sodium sulfate (anh.) and the solvent removed to give the product 2.02 g (17% yield).

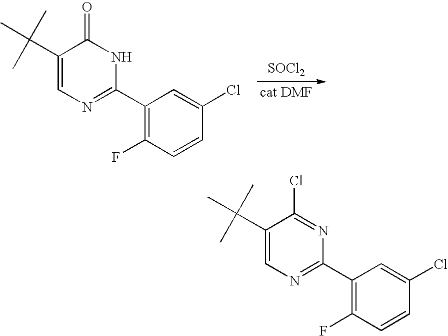

2-(5-chloro-2-fluorophenyl)-5-t-butylprimidine-4-one (2.02, 7.20 mmole) was suspended in thionyl chloride (10 ml) and 3 drops DMF were added. The mixture was heated to 80° C. for 30 min, removed excess thionyl chloride under vacuum. The residue was treated with ice (50 ml) and chloroform (50 ml). Extracted product into chloroform. Washed chloroform with 10% sodium carbonate (cold) and dried chloroform layer over sodium sulfate (anh.) Removed solvent to give 2.00 g product. (93% yield)

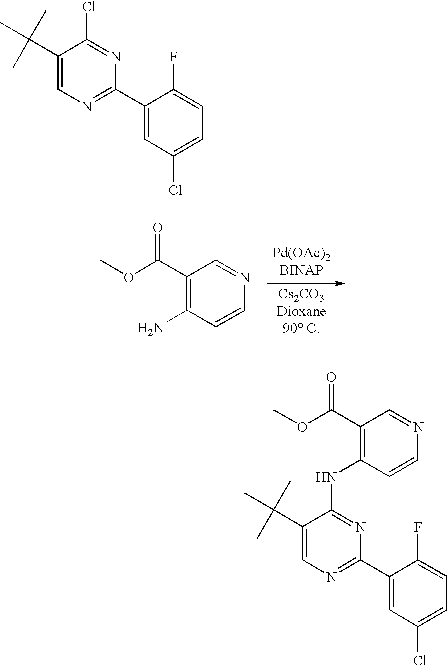

BINAP (311 mg, 0.50 mmol) and palladium(II)acetate (74 mg, 0.334 mmol were combined in 10 ml dioxane (anh.) and heated for 5 min, followed by addition of 2-(5-chloro-2-fluorophenyl)-4-chloro-5-t-butyllpyrimidine (2.00 g, 6.68 mmol), methyl 4-amino-3-pyridinecarboxylate (1.22 g, 8.0 mmol) and cesium carbonate (3.05 g, 9.38 mmol). The mixture was heated to 90° C. overnight. Removed dioxane under vacuum, the solid residue was triturated with ethyl acetate (20 ml) and filtered to give 3.15 g product which contains cesium carbonate and was used directly in next step without further purification.

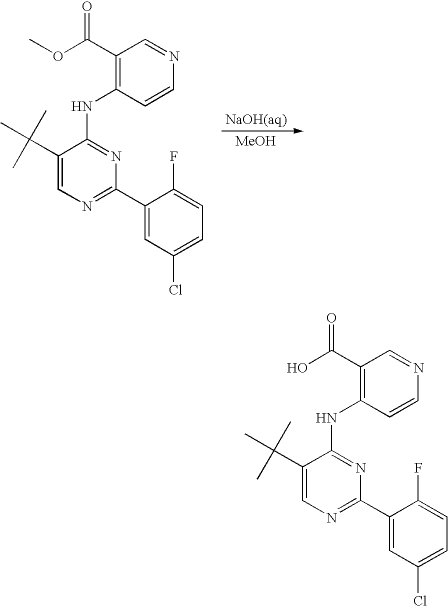

The ester (3.15 g,) was suspended in 10 ml methanol and treated with 4 ml 2.0M NaOH (aq). The mixture was refluxed for 1 hour, then the cooled reaction mixture was concentrated under vacuum to remove methanol. The aqueous solution was acidified with 6M HCl (pH 5), and filtered to obtain product 2.25 g.

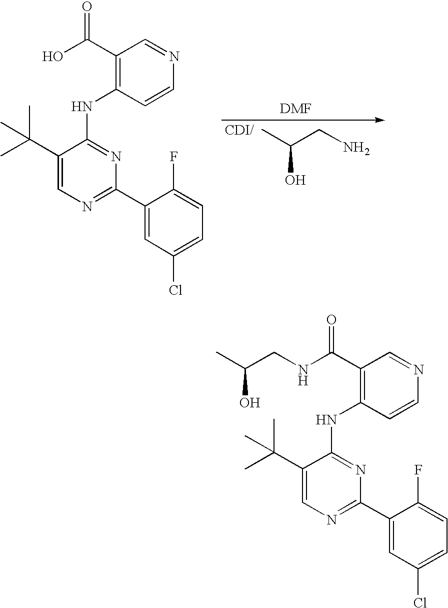

The acid (100 mg, 0.25 mmole) was suspended in DMF (anh., 3 ml) and treated with carbonyl diimidazole (81 mg, 0.5 mmole) and heated to 60° C. for 2 hours. S(+)-1-amino-2-propanol (75 mg, 1.0 mmole) was added and the solution was stirred overnight at room temperature. Filtered the mixture, and the filtrate was subjected to HPLC purification. Isolated 12 mg product.

### Example 10

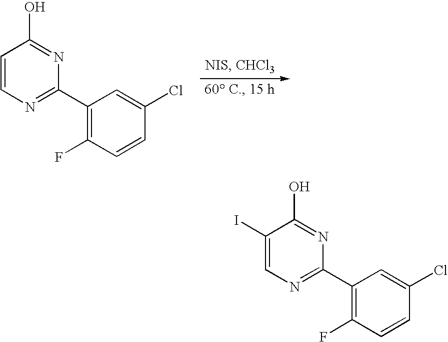

2-(5-Chloro-2-fluorophenyl)-5-iodopyrimidin-4-ol. To a solution of pyrimidone, 2-(5-chloro-2-fluorophenyl)-pyrimidin-4-ol (3.65 g, 16 mmol, 1 eq), in dry chloroform was added N-halosuccinimide, NIS (5.5 g, 24 mmol, 1.5 eq) in one portion and the reaction mixture was heated to 60° C. overnight. The reaction mixture was cooled to rt and partitioned between chloroform and water. The organic layers were combined, washed with brine, dried over MgSO₄, filtered and concentrated in vacuo and the residue was purified by flash column chromatography to give 2-(5-Chloro-2-fluorophenyl)-5-iodopyrimidin-4-ol (4.82 g, 84%) as a cream colored solid.

The product was converted into a compound of formula (I) by the methods in Example 3 above.

### Example 11

2-Cyclopentyl-3-oxo-propionic acid methyl ester. To a solution of diisopropylamine (18.92 g, 0.135 mole) in tetrahydrofuran (40 ml) at −20° C. was added n-butyl lithium (2.5M hexanes, 59 ml, 0.147 mole) dropwise. The solution stirred for 40 minutes at 0° C. The mixture was cooled to −78° C. and cyclopentyl-acetic acid methyl ester (17.52 g, 0.123 mole) was added dropwise. The reaction mixture continued to stir at −78° C. for 30 min. Ethyl formate (9.66 ml., 0.123 mole) was added and the reaction mixture was allowed to warm to room temperature while stirring for 18 hours. The reaction mixture was poured into ice water (300 ml). The organic phase was extracted with sodium hydroxide (1M, 2×40 ml) and the aqueous layers were combined. The cooled aqueous solution was acidified with 40% sulfuric acid to pH 5. The mixture was extracted with diethyl ether (5×40 ml), and the combined extracts were washed with saturated sodium chloride solution, and dried over anhydrous sodium sulfate. The organic solvent was removed under reduced pressure to give a 2-Cyclopentyl-3-oxo-propionic acid methyl ester as slightly yellow liquid, 16.24 g (78% yield). This material was used in the next step without further purification.

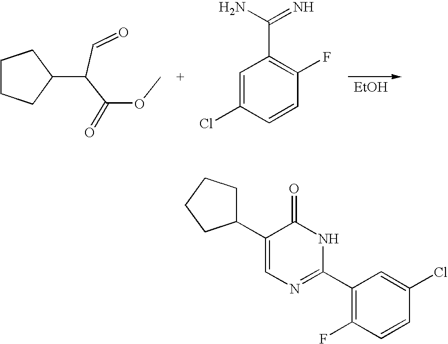

6-(5-Chloro-2-fluoro-phenyl)-3-cyclopentyl-1H-pyrimidin-2-one. The beta-aldehyde ester, 2-Cyclopentyl-3-oxo-propionic acid methyl ester (16.24 g, 95 mmol) and benzamidine, 5-chloro-2-fluorobenzamidine (16.39 g, 95 mmol) were combined in ethanol (120 ml) and heated to 80° C. for 18 hours. Ethanol was removed under reduced pressure and chloroform (400 mL) was added followed by 1M sodium hydroxide (100 ml). The aqueous layer was washed with chloroform (2×50 ml), acidified with 1M hydrochloric acid and extracted with ethyl acetate. Much of the product solidified from solution and was isolated by filtration. Upon drying the ethyl acetate filtrate further 6-(5-Chloro-2-fluoro-phenyl)-3-cyclopentyl-1H-pyrimidin-2-one was obtained after removal of the solvent under reduced pressure to afford 14.37 g (51% yield).

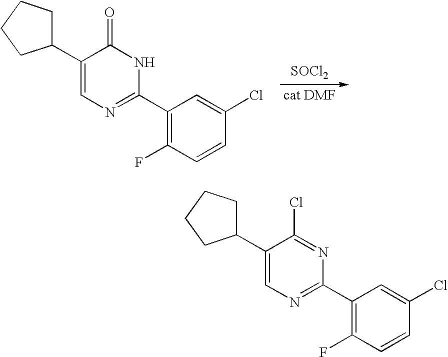

4-Chloro-2-(5-chloro-2-fluoro-phenyl)-5-cyclopentyl-pyrimidine. Pyrimidone, 6-(5-Chloro-2-fluoro-phenyl)-3-cyclopentyl-1H-pyridin-2-one (4.95 g, 16.91 mmole) was treated with thionyl chloride (20 ml). Dimethylformamide (3 drops) was added and the mixture was heated to reflux for 45 minutes. Excess thionyl chloide was removed under reduced pressure and the residue was combined with ice (˜100 g), chloroform (100 ml) and extracted product into chloroform layer. The chloroform extract was washed with 10% sodium carbonate, dried over anhydrous sodium sulfate and the solvent was removed under reduced pressure. This material was further purified by column chromatography over silica gel (chloroform). Obtained 4-Chloro-2-(5-chloro-2-fluoro-phenyl)-5-cyclopentyl-pyrimidine (5.00 g, Yield: 95%)

The compounds prepared by the methods described above can, of course, be further modified using methods known in the art. The following examples illustrate particular embodiments of such further transformations, but are offered as examples only and in no way limit the scope of the invention.

### Example 12

### Derivatization of Compounds Made by the Preceding Methods

### Synthesis of 4-[2-(5-Chloro-2-fluoro-phenyl)-5-methoxy-pyrimidin-4-ylamino]-N-[3-(3-isopropyl-ureido)-propyl]-nicotinamide

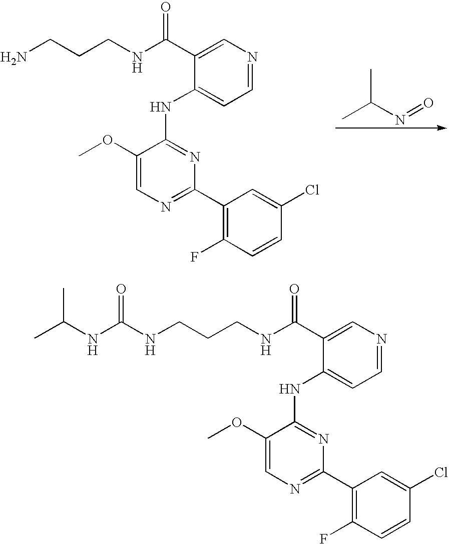

To a solution of N-(3-amino-propyl)-4-[2-(5-chloro-2-fluoro-phenyl)-5-methoxy-pyrimidin-4-ylamino]-nicotinamide (50 mg, 0.1163 mmol) in EtOAc (5 ml) was added triethyl amine (17 ul, 0.1163 mmol) and isoproyl isocyanate (0.1163 mmol). The reaction solution was stirred at room temperature overnight and a precipitate formed. The solvent was removed in vacu and the solid residue was rinsed with MeOH. 4-[2-(5-Chloro-2-fluoro-phenyl)-5-methoxy-pyrimidin-4-ylamino]-N-[3-(3-isopropyl-ureido)-propyl]-nicotinamide (20 mg) was obtained as a solid.

### Example 13

### Synthesis of 4-[2-(5-Chloro-2-fluoro-phenyl)-5-methoxy-pyrimidin-4-ylamino]-N-[3-(2-methyl-butyrylamino)-propyl]-nicotinamide

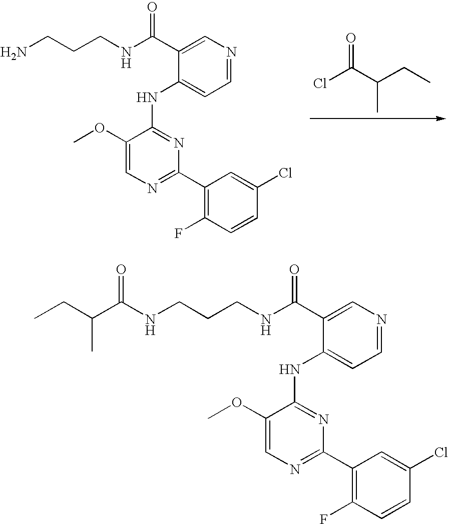

4-[2-(5-Chloro-2-fluoro-phenyl)-5-methoxy-pyrimidin-4-ylamino]-N-[3-(2-methyl-butyrylamino)-propyl]-nicotinamide. To solution of N-(3-amino-propyl)-4-[2-(5-chloro-2-fluoro-phenyl)-5-methoxy-pyrimidin-4-ylamino]-nicotinamide (50 mg, 0.1163 mmol) in DMF (5 ml) was added isobutyryl chloride (24 ul, 0.2326 mmol). The reaction solution was stirred at ambient temperature overnight. The product was purified by prep HPLC after removal of solvent to afford 4-[2-(5-chloro-2-fluoro-phenyl)-5-methoxy-pyrimidin-4-ylamino]-N-[3-(2-methyl-butyrylamino)-propyl]-nicotinamide (16% yield).

### Example 14

### Synthesis of 4-[2-(5-chloro-2-fluoro-phenyl)-5-methoxy-5,6-dihydro-pyrimidin-4-ylamino]-N-{3-[(cyanoimino-isopropylamino-methylene)-amino]-propyl}-nicotinamide

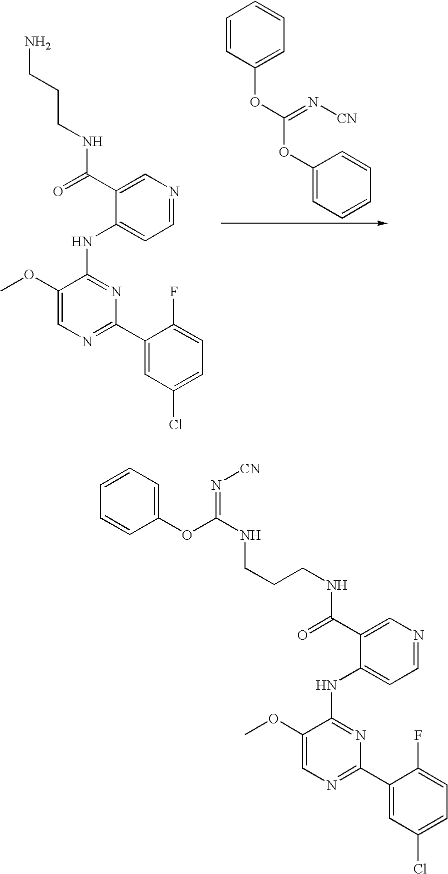

4-[2-(5-Chloro-2-fluoro-phenyl)-5-methoxy-5,6-dihydro-pyrimidin-4-ylamino]-N-{3-[(cyanoimino-phenoxy-methylene)-amino]-propyl}-nicotinamide. The N-(3-amino-propyl)-4-[2-(5-chloro-2-fluoro-phenyl)-5-methoxy-5,6-dihydro-pyrimidin-4-ylamino]-nicotinamide (0.200 g) was dissolved in 2-propanol (20 mL) and diphenoxymethylene-cyanamine (0.115) was added. The mixture stirred at 70° C. for 8 h and then was cooled to rt. The mixture was filtered and the solid material was filtered 4-[2-(5-chloro-2-fluoro-phenyl)-5-methoxy-5,6-dihydro-pyrimidin-4-ylamino]-N-{3-[(cyanoimino-phenoxy-methylene)-amino]-propyl}-nicotinamide (0.160 mg) and used in the next reaction without further purification.

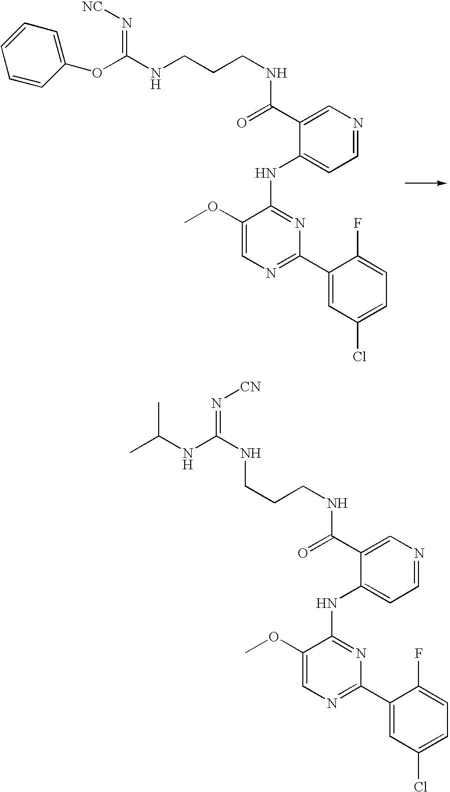

4-[2-(5-Chloro-2-fluoro-phenyl)-5-methoxy-5,6-dihydro-pyrimidin-4-ylamino]-N-{3-[(cyanoimino-isopropylamino-methylene)-amino]-propyl}-nicotinamide. To a solution of collected 4-[2-(5-chloro-2-fluoro-phenyl)-5-methoxy-5,6-dihydro-pyrimidin-4-ylamino]-N-{3-[(cyanoimino-phenoxy-methylene)-amino]-propyl}-nicotinamide (0.050 g) in 2-propanol (5 mL) was added iso-propyl amine (5 equivalents). The mixture stirred at rt for 5 days in a sealed flask. The reaction was reduced in volumn and filtered to afford the desired product 4-[2-(5-chloro-2-fluoro-phenyl)-5-methoxy-5,6-dihydro-pyrimidin-4-ylamino]-N-{3-[cyanoimino-isopropylamino-isopropylamino-methylene)-amino]-propyl}-nicotinamide (3.7 mg).

### Example 15

4-({4-[2-(5-Chloro-2-fluoro-phenyl)-5-methoxy-pyrimidin-4-ylamino]-pyridine-3-carbonyl}-amino)-pyrrolidine-1,2-dicarboxylic acid 1-tert-butyl ester 2-methyl ester. Synthesized as described in Example 3, using N1-BOC protected 4-aminoproline methyl ester.

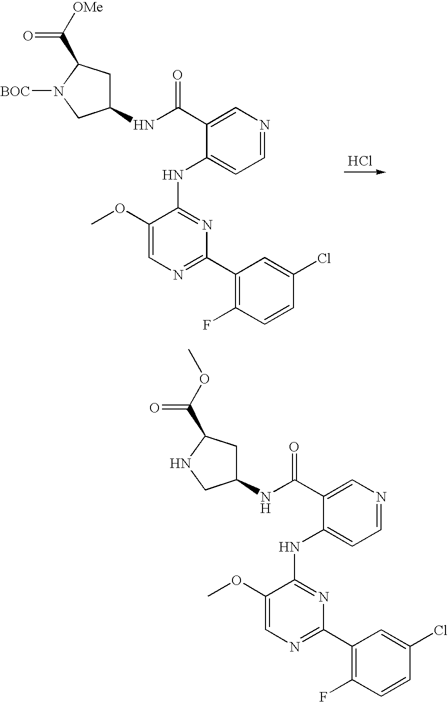

4-({4-[2-(5-Chloro-2-fluoro-phenyl)-5-methoxy-pyrimidin-4-ylamino]-pyridine-3-carbonyl}-amino)-pyrrolidine-2-carboxylic acid methyl ester. The BOC-protected amine, 4-({4-[2-(5-chloro-2-fluoro-phenyl)-5-methoxy-pyrimidin-4-ylamino]-pyridine-3-carbonyl}-amino-pyrrolidine-1,2-dicarboxylic acid 1-tert-butyl ester 2-methyl ester (50 mg) in 4M HCL dioxane (2 mL) was stirred for 4 hours. The solvent was removed in vacuo. Purification by preparative HPLC (5/70 water/acetonitrile/20 mins) afforded 4-({4-[2-(5-chloro-2-fluoro-phenyl)-5-methoxy-pyrimidin-4-ylamino]-pyridine-3-carbonyl}-amino)-pyrrolidine-2-carboxylic acid methyl ester (25 mg, 0.050 mmol; 50% yield).

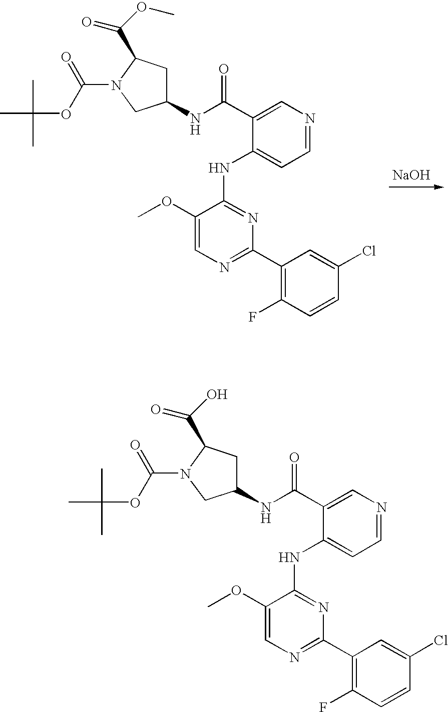

4-({4-[2-(5-Chloro-2-fluoro-phenyl)-5-methoxy-pyrimidin-4-ylamino]-pyridine-3-carbonyl}-amino)-pyrrolidine-1,2-dicarboxylic acid 1-tert-butyl ester. A solution of ester, 4-({4-[2-(5-Chloro-2-fluoro-phenyl)-5-methoxy-pyrimidin-4-ylamino]-pyridine-3-carbonyl}-amino)-pyrrolidine-1,2-dicarboxylic acid 1-tert-butyl ester 2-methyl ester (400 mg) in 1 M NaOH (1 mL) and dioxane (6 mL) was heated at 60° C. for 2.5 hours. 1 M HCl (2 mL) was added and the reaction mixture partitioned between water (50 mL) and CH₂Cl₂ (50 mL). The aqueous layer was further extracted with CH₂Cl₂ (3×50 mL) and the extracts were combined and the solvent was removed in vacuo. Purification by preparative HPLC (5/95 water/acetonitrile/20 mins) afforded 4-({4-[2-(5-chloro-2-fluoro-phenyl)-5-methoxy-pyrimidin-4-ylamino]-pyridine-3-carbonyl}-amino)-pyrrolidine-1,2-dicarboxylic acid 1-tert-butyl ester (110 mg, 0.187 mmol 28% yield).

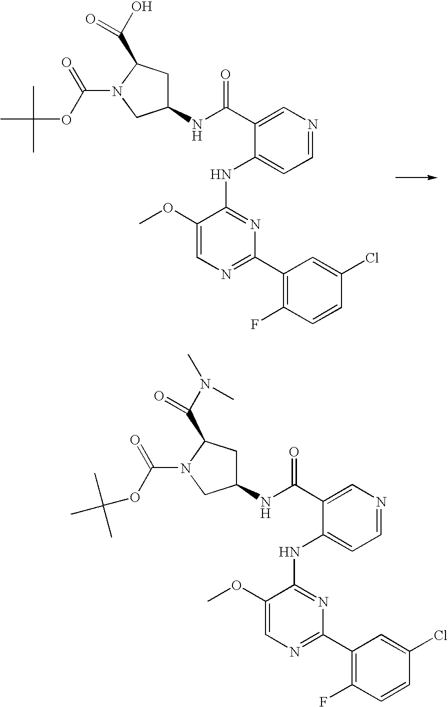

The dimethyl amide was formed from the carboxylic acid as described in Example 3 above.

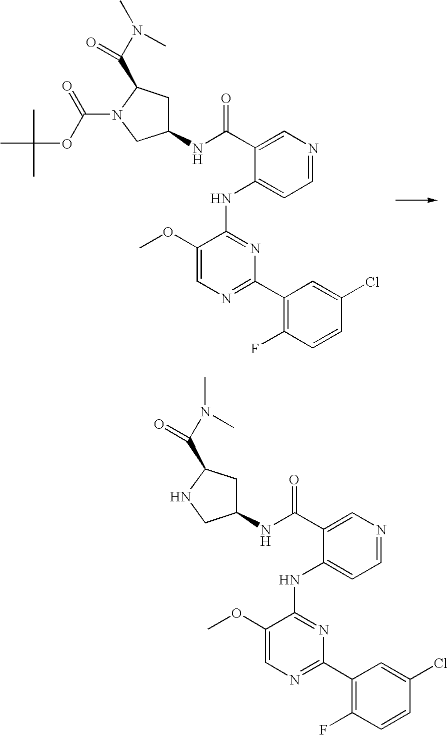

4-[2-(5-Chloro-2-fluoro-phenyl)-5-methoxy-pyrimidin-4-ylamino]-N-(5-dimethylcarbamoyl-pyrrolidin-3-yl)-nicotinamide. The Boc-protected amine, 4-({4-[2-(5-chloro-2-fluoro-phenyl)-5-methoxy-pyrimidin-4-ylamino]-pyridine-3-carbonyl}-amino)-2-dimethylcarbamoyl-pyrrolidine-1-carboxylic acid tert-butyl ester (48 mg, 0.078 mmol) in 4 M HCL dioxane (2 mL) was stirred at room temperature for 3 hours. The solvent was removed in vacuo. Re-dissolved in DMF and purified by preparative HPLC (5/70 water/acetonitrile/20 mins) to afford 4-[2-(5-chloro-2-fluoro-phenyl)-5-methoxy-pyrimidin-4-ylamino]-N-(5-dimethylcarbamoyl-pyrrolidin-3-yl)-nicotinamide (26 mg, 0.051 mmol 65%).

### Example 16

### Preparation of a Pyridine N-Oxide Compound

Synthesis of 4-[2-(5-Chloro-2-fluoro-phenyl)-5-methoxy-pyrimidin-4-ylamino]-N-cyclopropyl-1-oxy-nicotinamide.

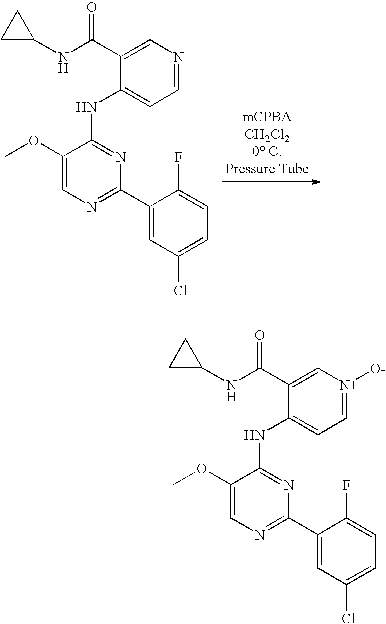

4-[2-(5-Chloro-2-fluoro-phenyl)-5-methoxy-pyrimidin-4-ylamino]-N-cyclopropyl-1-oxy-nicotinamide. To a 350 mL round pressure vessel was added 4-[2-(5-chloro-2-fluoro-phenyl)-5-methoxy-pyrimidin-4-ylamino]-N-cyclopropyl-nicotinamide (1.28 g, 3.105 mmol) followed by methylene chloride (30 ml). The flask was placed in an ice-bath at 0° C. While maintaining the temperature at 0-2° C. mCPBA (2.15 g of 77%, 9.6 mmol) was added and the reaction mixture was allowed to stir in the sealed reaction flask. After 2 hours the reaction was quenched by adding of saturated sodium bicarbonate (30 mls) and extracted with dichloromethane (2×50 ml). The organic layer, which contained suspended solid, was separated, filtered and washed with acetone (3×50 ml). The remaining bright yellow solid contained 4-[2-(5-chloro-2-fluoro-phenyl)-5-methoxy-pyrimidin-4-ylamino]-N-cyclopropyl-1-oxy-nicotinamide was purified by HPLC (yield=65.8%).

### Example 17

### Activity of Selected Compounds of the Invention

The compounds of the invention were tested for their ability to inhibit TGFβ by a TGFβ R¹ autophosphorylation protocol. This was conducted as follows: Compound dilutions and reagents were prepared fresh daily. Compounds were diluted from DMSO stock solutions to 2 times the desired assay concentration, keeping final DMSO concentration in the assay less than or equal to 1%. TGFβ R1 was diluted to 4 times the desired assay concentration in buffer+DTT. ATP was diluted into 4× reaction buffer, and gamma-³³P-ATP was added at 60 uCi/mL.

The assay was performed by adding 10 ul of the enzyme to 20 ul of the compound solution. The reaction was initiated by the addition of 10 ul of ATP mix. Final assay conditions included 10 uM ATP, 170 nM TGFβ R1, and 1M DTT in 20 mM MOPS, pH7. The reactions were incubated at room temperature for 20 minutes. The reactions were stopped by transferring 23 ul of reaction mixture onto a phosphocellulose 96-well filter plate, which had been pre-wetted with 15 ul of 0.25M H₃PO₄ per well. After 5 minutes, the wells were washed 4× with 75 mM H₃PO₄ and once with 95% ethanol. The plate was dried, scintillation cocktail was added to each well, and the wells were counted in a Packard TopCount microplate scintillation counter.

Table 1 provides activity and structure characterization data for numerous compounds of formula (1) as described herein. The compounds in Table 1 are characterized in part by their biological activity and in part by their structure: the compounds were characterized in part by LC-mass spectrometry, and the second column of the Table provides the observed parent ion that was observed in the LC-MS analysis of the compounds that were prepared by the methods described above. In each case, the expected parent ion was observed, and the Table further provides the LC conditions under which the mass spectrum was measured as well as the retention time of the observed product.

The compounds in Table 1 provide, in this assay, IC₅₀ values in the range of less than 0.01 to about 20 micromolar. Many compounds, as indicated in the Table, had activity well below 1 micromolar. The invention expressly includes the compounds set forth in Table 1, as well as their pharmaceutically acceptable salts and pharmaceutical compositions containing them. The compounds in Table 1 are only examples of the invention, however, and are not intended to limit its scope. Accordingly, compounds that comprise combinations of the various features illustrated by the examples in the Table as representative of R¹, W, Ar, Y and Z in the Table are within the scope of the invention even if not expressly set forth.

TABLE 1

| | | m/z (M + H⁺), | IC₅₀ for |
|---|---|---|---|
| | | HPLC retention time | Kinase |
| No. | Structure | (min)* | Inhibition |
| 1 | 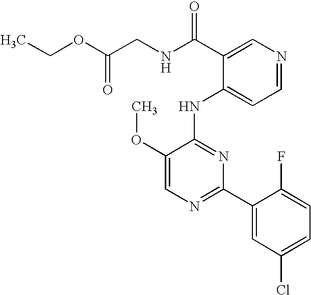 | 460.1, 1.360 | 0.04 |
| | AutoNom Name: | | |
| | ({4-[2-(5-Chloro-2-fluoro-phenyl)-5- | | |
| | methoxy-pyrimidin-4-ylamino]pyridine- | | |
| | 3-carbonyl}-amino)-acetic acid | | |
| | ethyl ester | | |
| 2 | 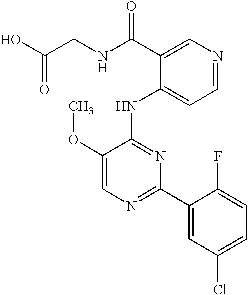 | 432.2, 1.213 | 0.92 |
| | AutoNom Name: | | |
| | ({4-[2-(5-Chloro-2-fluoro-phenyl)-5- | | |
| | methoxy-pyrimidin-4-ylamino]-pyridine | | |
| | 3-carbonyl}-amino)-acetic acid | | |
| 3 | 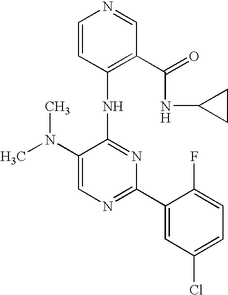 | | |
| | AutoNom Name: | | |
| | 4-[2-(5-Chloro-2-fluoro-phenyl)-5- | | |
| | dimethylamino-pyrimidin-4-ylamino]-1- | | |
| | cyclopropyl-nicotinamide | | |
| 4 | 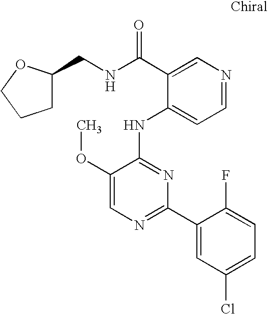 | | |
| | AutoNom Name: | | |
| | 4-[2 -(5-Chloro-2-fluoro-phenyl)-5- | | |
| | methoxy-pyrimidin-4-ylamino]-N-(tetrahydro- | | |
| | furan-2-ylmethyl)-nicotinamide | | |
| 5 | 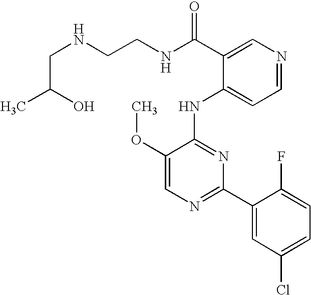 | 475.1, 1.11 | 0.02 |
| | AutoNom Name: | | |
| | 4-[2-(5-Chloro-2-fluoro-phenyl)-5- | | |
| | methoxy-pyrimidin-4-ylamino]-N-[2-(2- | | |
| | hydroxy-propylamino)-nicoti- | | |
| | namide | | |
| 6 | 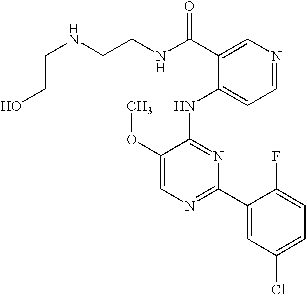 | 461.1,1.07 | 0.01 |
| | AutoNom Name: | | |
| | 4-[2-(5-Chloro-2-fluoro-phenyl)-5- | | |
| | methoxy-pyrimidin-4-ylamino]-N-[2-(2- | | |
| | hydroxy-ethylamino)-ethyl]-nicotin- | | |
| | amide | | |
| 7 | 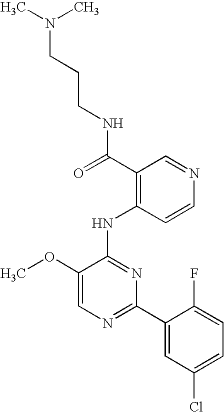 | 415.29, 2.46^{a} | 0.03 |
| | AutoNom Name: | | |
| | 4-[2-(5-Chloro-2-fluoro-phenyl)-5- | | |
| | methoxy-pyrimidin-4-ylamino]-N-(3-di- | | |
| | methylamino-propyl)-nicotinamide | | |
| 8 | 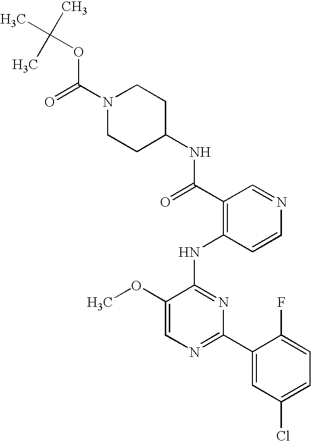 | 457.16, 2.46^{a} | 0.06 |
| | AutoNom Name: | | |
| | 4-({4-[2-(5-Chloro-2-fluoro-phenyl)- | | |
| | 5-methoxy-pyrimidin-4-ylamino]- | | |
| | pyridine-3-carbonyl}-amino)-piperidine- | | |
| | 1-carboxylic acid tert-butyl ester | | |
| 9 | 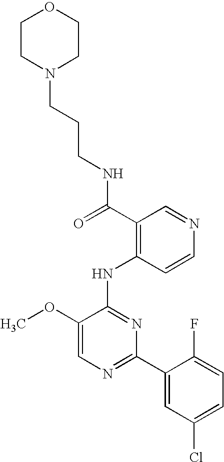 | 501.17, 2.36^{a} | 0.03 |
| | AutoNom Name: | | |
| | 4-[2-(5-Chloro-2-fluoro-phenyl)-5- | | |
| | methoxy-pyrimidin-4-ylamino]-N-(3- | | |
| | morpholin-4-yl-propyl)-nicotinamide | | |
| 10 | 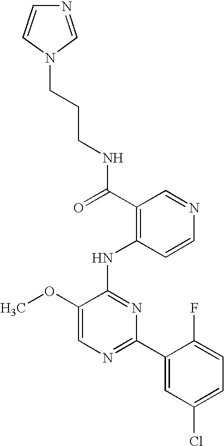 | 482.15, 2.36^{a} | 0.06 |
| | AutoNom Name: | | |
| | 4-[2-(5-Chloro-2-fluoro-phenyl)-5- | | |
| | methoxy-pyrimidin-4-ylamino]-N-(3- | | |
| | inidazol-1-yl-propyl)-nicotinamide | | |
| 11 | 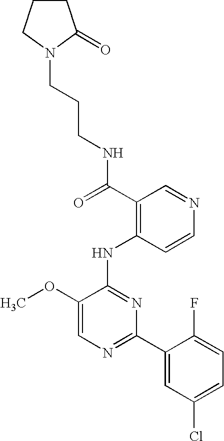 | 499.19, 2.78^{a} | 0.12 |
| | AutoNom Name: | | |
| | 4-{2-(5-Chloro-2-fluoro-phenyl)-5- | | |
| | methoxy-pyrimidin-4-ylamino]-N-[3-(2- | | |
| | oxo-pyrrolidin-1-yl)-propyl]-nico- | | |
| | tinamide | | |
| 12 | 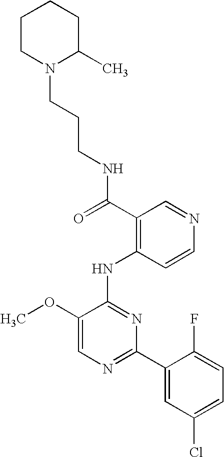 | 513.22, 2.51^{a} | 0.06 |
| | AutoNom Name: | | |
| | 4-[2-(5-Chloro-2-fluoro-phenyl)-5- | | |
| | methoxy-pyrimidin-4-ylamino]-N-[3-(2- | | |
| | methyl-piperidin-1-yl)-propyl]-nic- | | |
| | otinamide | | |
| 13 | 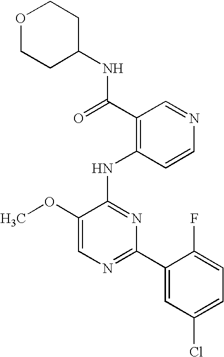 | | |
| | AutoNom Name: | | |
| | 4-[2-(5-Chloro-2-fluoro-phenyt)-5- | | |
| | methoxy-pyrimidin-4-ylamino]-N-(tetra- | | |
| | ahydro-pyran-4-yl)-nicotinamide | | |
| 14 | 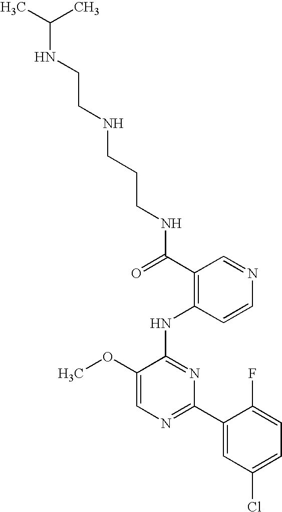 | 502.15, 2.28^{a} | 0.12 |
| | AutoNom Name: | | |
| | 4-[2-(5-Chloro-2-fluoro-phenyl)-5- | | |
| | methoxy-pyrimidin-4-ylamino]-N-[3-(2- | | |
| | isopropylamino-ethylamino)-propyl- | | |
| | nicotinamide | | |
| 15 | 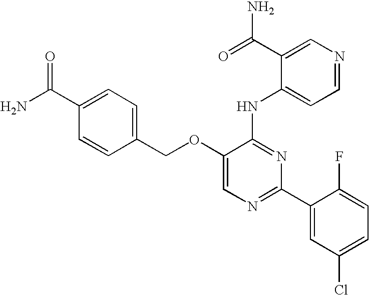 | | |
| | AutoNom Name: | | |
| | 4-[5-(4-Carbamoyl-benzyloxy)-2-(5- | | |
| | chloro-2-fluoro-phenyl)-pyrimidin-4- | | |
| | ylamino)-nicotinamide | | |
| 16 | 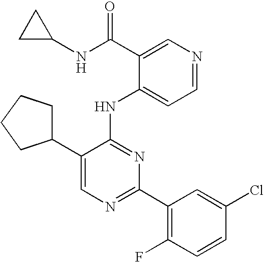 | | |
| | AutoNom Name: | | |
| | 4-[2-(5-Chloro-2-fluoro-phenyl)-5- | | |
| | cyclopentyl-pyrimidin-4-ylamino]-N- | | |
| | yclopropyl-nicotinamide | | |
| 17 | 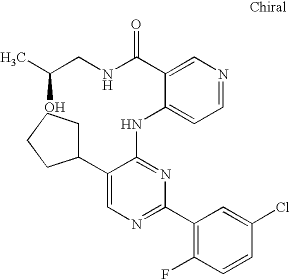 | | |
| | AutoNom Name: | | |
| | 4-[2-(5-Chloro-2-fluoro-phenyl)-5- | | |
| | cyclopentyl-pyrimidin-4-ylamino]-N- | | |
| | (2-hydroxy-propyl)-nicotinamide | | |
| 18 | 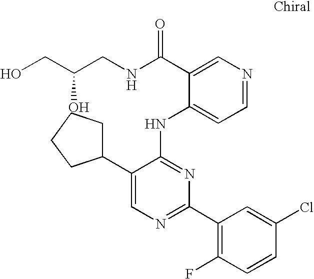 | | |
| | AutoNom Name: | | |
| | 4-[2-(5-Chloro-2-fluoro-phenyl)-5- | | |
| | cyclopentyl-pyrimidin-4-ylamino)-N- | | |
| | (2,3-dihydroxy-propyl)-nicotinamide | | |
| 19 | 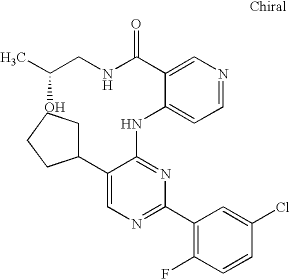 | | |
| | AutoNom Name: | | |
| | 4-[2-(5-Chloro-2-fluoro-phenyl)-5- | | |
| | cyclopentyl-pyrimidin-4-ylamino]-N- | | |
| | (2-hydroxy-propyl)-nicotinamide | | |
| 20 | 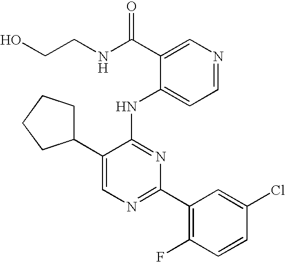 | | |
| | AutoNom Name: | | |
| | 4-[2-(5-Chloro-2-fluoro-phenyl)-5- | | |
| | cyclopentyl-pyrimidin-4-ylamino]-N- | | |
| | 2-hydroxy-ethyl)-nicotinamide | | |
| 21 | 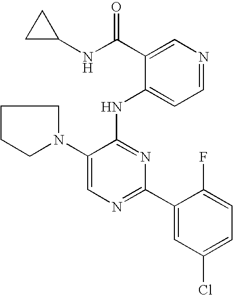 | 453.1, 1.527 | 0.18 |
| | AutoNom Name: | | |
| | 4-[2-(5-Chloro-2-fluoro-phenyl)-5- | | |
| | pyrrolidin-1-yl-pyrimidin-4-ylamino]- | | |
| | N-cyclopropyl-nicotinamide | | |
| 22 | 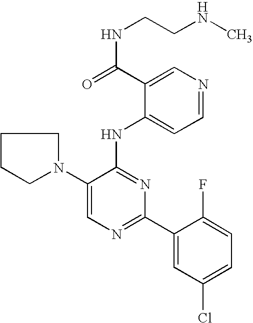 | 470.2, 1.233 | 0.10 |
| | AutoNom Name: | | |
| | 4-[2-(5-Chloro-2-fluoro-phenyl)-5- | | |
| | pyrrolidin-1-yl-pyrimidin-4-ylamino]- | | |
| | N-(2-methylamino-ethyl)-nicotinamide | | |
| 23 | 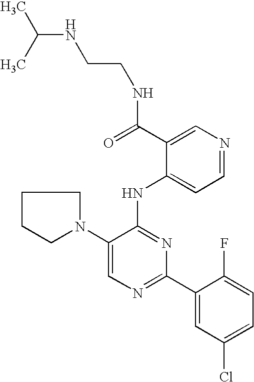 | 498.3, 1.287 | 0.20 |
| | AutoNom Name: | | |
| | 4-[2-(5-Chloro-2-fluoro-phenyl)-5- | | |
| | pyrrolidin-1-yl-pyrimidin-4-ylamino]- | | |
| | N-(2-isopropylamino-ethyl)-nicotinamide | | |
| 24 | 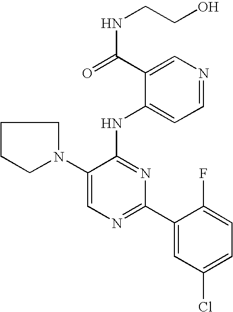 | 457.3, 1.353 | 0.49 |
| | AutoNom Name: | | |
| | 4-[2-(5-Chloro-2-fluoro-phenyl)-5- | | |
| | pyrrolidin-1-yl-pyrimidin-4-ylamino]- | | |
| | N-(2-hydroxy-ethyl)-nicotinamide | | |
| 25 | 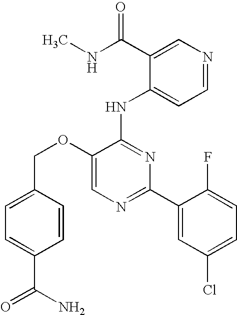 | | |
| | AutoNom Name: | | |
| | 4-[5-(4-Carbamoyl-benzyloxy)-2-(5- | | |
| | chloro-2-fluoro-phenyl)-pyrimidin-4- | | |
| | ylamino]-N-methyl-nicotinamide | | |
| 26 | 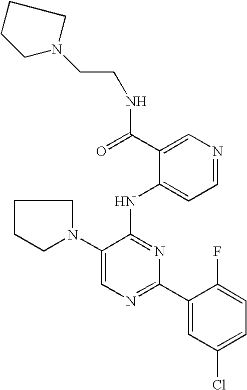 | 510.3,1.240 | 0.20 |
| | AutoNom Name: | | |
| | 4-[2-(5-Chloro-2-fluoro-phenyl)-5- | | |
| | pyrrolidin-1-yl-pyrimidin-4-ylamino]- | | |
| | N-(2-pyrrolidin-1-yl-ethyl)-nicotinamide | | |
| 27 | 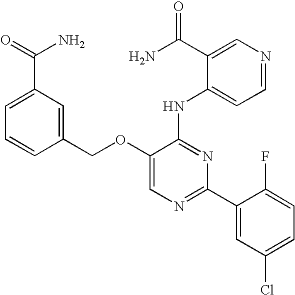 | | |
| | AutoNom Name: | | |
| | 4-[5-(3-Carbamoyl-benzyloxy)-2-(5- | | |
| | chloro-2-fluoro-phenyl)-pyrimidin-4- | | |
| | ylamino]-nicotinamide | | |
| 28 | 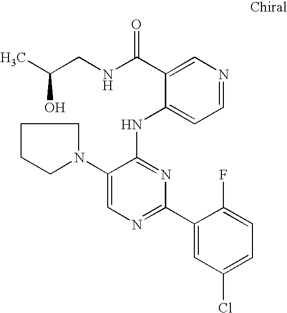 | 471.1,1.473 | 547 |
| | AutoNom Name: | | |
| | 4-[2-(5-Chloro-2-fluoro-phenyl)-5- | | |
| | pyrrollidin-1-yl-pyrimidin-4-ylamino]- | | |
| | N-(2-hydroxy-propyl)-nicotinamide | | |
| 29 | 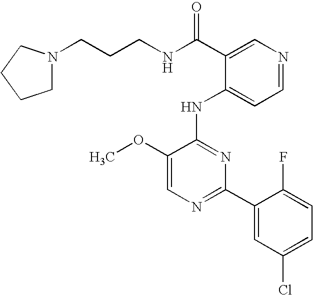 | 485.4, 1.15 | 0.03 |
| | AutoNom Name: | | |
| | 4-[2-(5-Chloro-2-fluoro-phenyl)-5- | | |
| | methoxy-pyrimidin-4-ylamino]-N-3- | | |
| | pyrrolidin-1-yl-propyl)-nicotinamide | | |
| 30 | 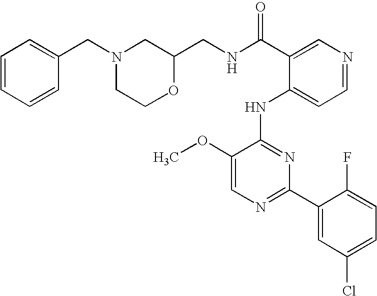 | 563.2, 1.32 | 2.37 |
| | AutoNom Name: | | |
| | N-(4-Benzyl-morpholin-2-ylmethyl)-4- | | |
| | [2-(5-chloro-2-fluoro-phenyl)-5- | | |
| | methoxy-pyrimidin-4-ylamino)-nicotinamide | | |
| 31 | 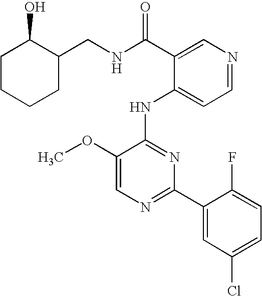 | 486.1, 1.433 | 0.37 |
| | AutoNom Name: | | |
| | 4-[2-(5-Chloro-2-fluoro-phenyl)-5- | | |
| | methoxy-pyrimidin-4-ylamino]-N-(2- | | |
| | hydroxy-cyclohexylmethyl)-nicotinamide | | |
| 32 | 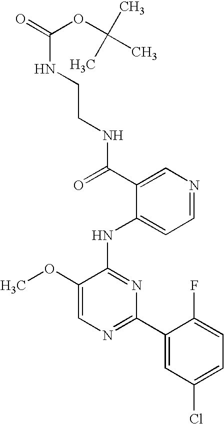 | 517.2, 1.53 | 0.56 |
| | AutoNom Name: | | |
| | [2-({4-[2-(5-Chloro-2-fluoro-phenyl)- | | |
| | 5-methoxy-pyrimidin-4-ylamino]- | | |
| | pyridine-3-carbonyl}-amino)-ethyl]- | | |
| | carbamic acid tert-butyl ester | | |
| 33 | 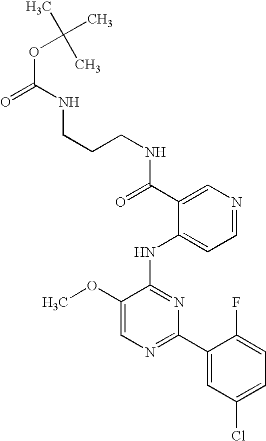 | 531.2, 1.58 | 1.13 |
| | AutoNom Name: | | |
| | [3-({4-[2-(5-Chloro-2-fluoro-phenyl)- | | |
| | 5-methoxy-pyrmidin-4-ylamino]- | | |
| | pyridine-3-carbonyl}-amino)-propyl]- | | |
| | carbamic acid tert-butyl ester | | |
| 34 | 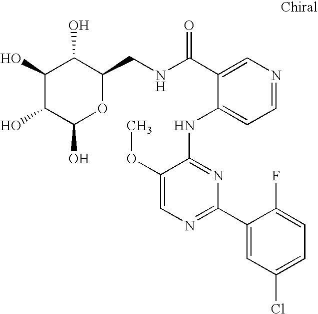 | 536.2, 1.113 | 1.16 |
| | AutoNom Name: | | |
| | 4-[2-(5-Chloro-2-fluoro-phenyl)-5- | | |
| | methoxy-pyrimidin-4-ylamino)-N-(3,4, | | |
| | 5,6-tetahydroxy-tetrahrydro-pyran-2- | | |
| | ylmethyl)-nicotinamide | | |
| 35 | 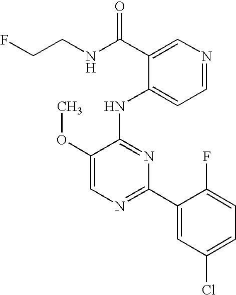 | | |
| | AutoNom Name: | | |
| | 4-[2-(5-chloro-2-fluoro-phenyl)-5- | | |
| | methoxy-pyrimidin-4-ylamino]-N-(2- | | |
| | fluoro-ethyl)-nicotinamide | | |
| 36 | 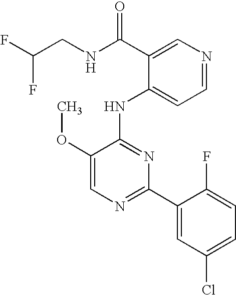 | 438.0, 1.327 | 0.67 |
| | AutoNom Name: | | |
| | 4-[2-(5-Chloro-2-fluoro-phenyl)-5- | | |
| | methoxy-pyrimidin-4-ylamino]-N-(2,2- | | |
| | difluoro-ethyl)-nicotinamide | | |
| 37 | 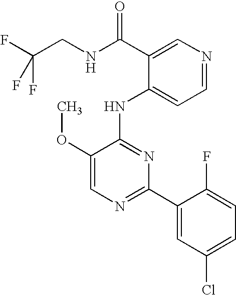 | 456.0, 1.393 | 0.16 |
| | AutoNom Name: | | |
| | 4-[2-(5-Chloro-2-fluoro-phenyl)-5- | | |
| | methoxy-pyrimidin-4-ylamino]-N-(2,2, | | |
| | 2-trifluoro-ethyl)-nicotinamide | | |
| 38 | 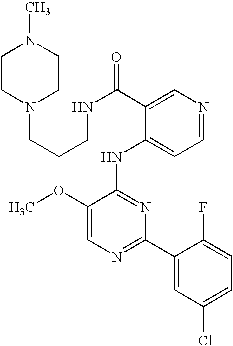 | 514.2, 1.053 | 0.54 |
| | AutoNom Name: | | |
| | 4-[2-(5-Chloro-2-fluoro-phenyl)-5- | | |
| | methoxy-pyrimidin-4-ylamino]-N-[3-(4- | | |
| | methyl-piperazin-1-yl)-propyl]- | | |
| | nicotinamide | | |
| 39 | 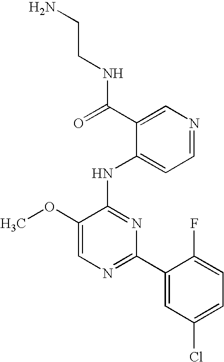 | 417.0,1.10 | 0.05 |
| | AutoNom Name: | | |
| | N-(2-Amino-ethyl)-4-[2-(5-chloro-2- | | |
| | fluoro-phenyl)-5-methoxy-pyrimidin- | | |
| | 4-ylamino]-nicotinamide | | |
| 40 | 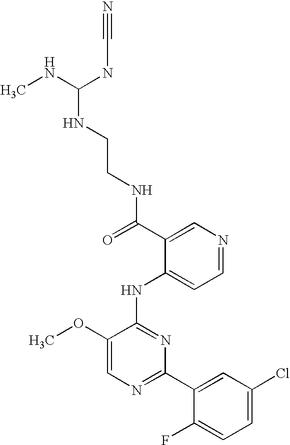 | 498.1, 1.23 | 0.14 |
| 41 | 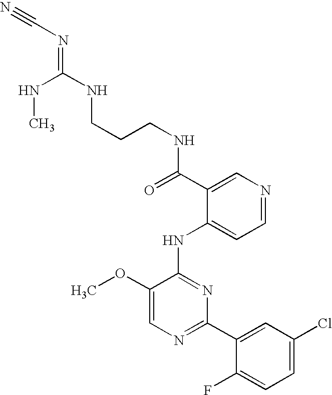 | 512.1, 1.25 | 0.05 |
| 42 | 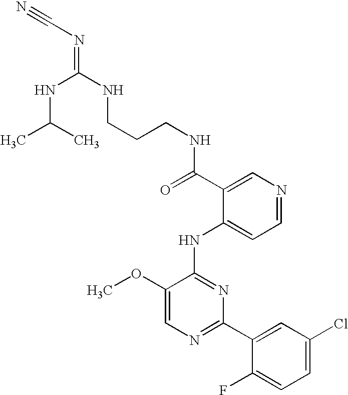 | 540.2, 1.33 | 0.12 |
| 43 | 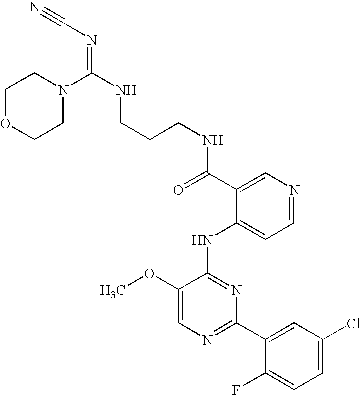 | 569.1, 1.29 | 0.06 |
| 44 | 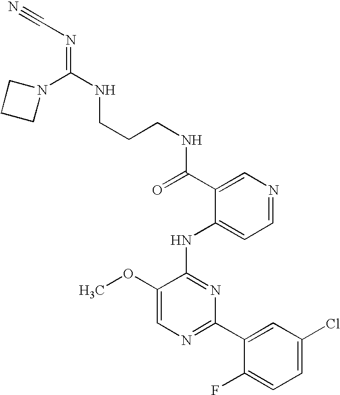 | 538.2, 1.29 | 0.13 |
| 45 | 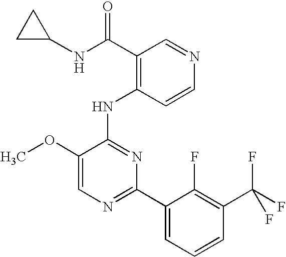 | | |
| | AutoNom Name: | | |
| | N-Cyclopropyl-4-[2-(2-fluoro-3- | | |
| | trifluoromethyl-phenyl)-5-methoxy- | | |
| | pyrimidin-4-ylamino]-nicotinamide | | |
| 46 | 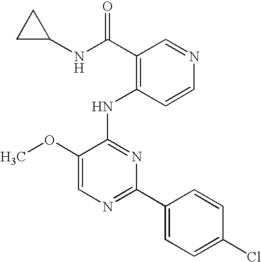 | | |
| | AutoNom Name: | | |
| | 4-[2-(4-Chloro-phenyl)-5-methoxy- | | |
| | pyrimidin-4-ylamino]-N-cyclopropyl- | | |
| | nicotinamide | | |
| 47 | 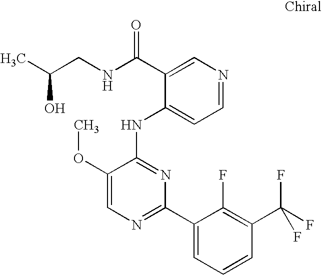 | | |
| | AutoNom Name: | | |
| | 4-[2-(2-Fluoro-3-trifluoromethyl- | | |
| | phenyl)-5-methoxy-pyrimidin-4-ylamino]- | | |
| | N-(2-hydroxy-propyl)-nicotinamide | | |
| 48 | 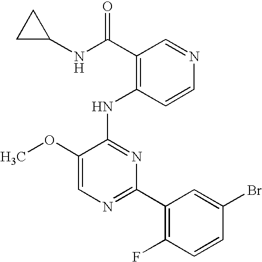 | | |
| | AutoNom Name: | | |
| | 4-[2-(5-Bromo-2-fluoro-phenyl)-5- | | |
| | methoxy-pyrimidin-4-ylamino]-N-cyclo- | | |
| | propyl-nicotinamide | | |
| 49 | 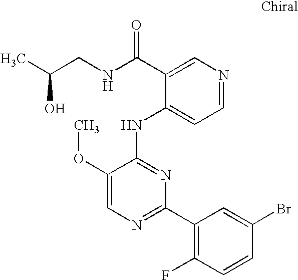 | | |
| | AutoNom Name: | | |
| | 4-[2-(5-Bromo-2-fluoro-phenyl)-5- | | |
| | methoxy-pyrimidin-4-ylamino]-N-(2- | | |
| | hydroxy-propyl)-nicotinamide | | |
| 50 | 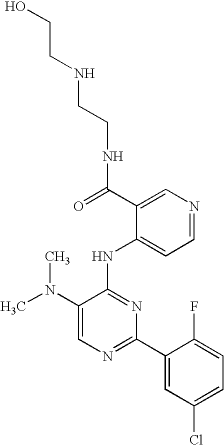 | 474.1, 1.133 | 0.02 |
| | AutoNom Name: | | |
| | 4-[2-(5-Chloro-2-fluoro-phenyl)-5- | | |
| | dimethylamino-pyrimidin-4-ylamino[-1- | | |
| | [2-(2-hydroxy-ethylamino)-ethyl]- | | |
| | nicotinamide | | |
| 51 | 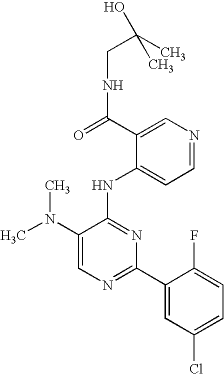 | 459.1, 1.420 | 0.12 |
| | AutoNom Name: | | |
| | 4-[2-(5-Chloro-2-fluoro-phenyl)-5- | | |
| | dimethylamino-pyrimidin-4-ylamino]-1- | | |
| | (2-hydroxy-2-methyl-propyl)-nicotinamide | | |
| 52 | 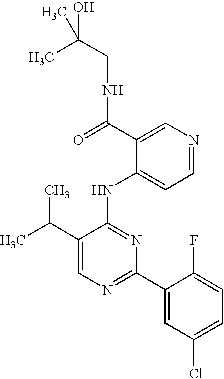 | 458.3, 1.387 | 0.09 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxy-2-methyl-propyl)-nicotinamide | | |
| 53 | 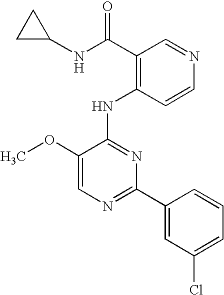 | | |
| | AutoNom Name: | | |
| | 4-[2-(3-Chloro-phenyl)-5-methoxy- | | |
| | pyrimidin-4-ylamino]-N-cyclopropyl- | | |
| | nicotinamide | | |
| 54 | 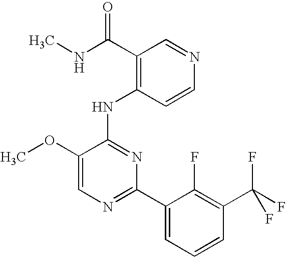 | | |
| | AutoNom Name: | | |
| | 4-[2-(2-Fluoro-3-trifluoromethyl- | | |
| | phenyl)-5-methoxy-pyrimidin-4-ylamino]- | | |
| | N-methyl-nicotinamide | | |
| 55 | 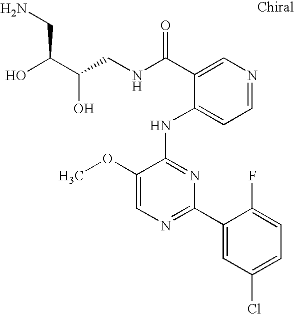 | 477.1, 1.160 | 0.13 |
| | AutoNam Name: | | |
| | N-(4-Amino-2,3-dihydroxy-butyl)-4-[ | | |
| | 2-(5-chloro-2-fluoro-phenyl)-5-methoxy- | | |
| | pyrimidin-4-ylamino]-nicotinamide | | |
| 56 | 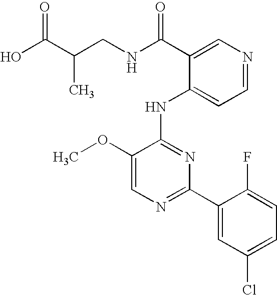 | 460, 1.67 | 1.20 |
| | AutoNom Name: | | |
| | 3-({4-[2-(5-Chloro-2-fluoro-phenyl)- | | |
| | 5-methoxy-pyrimidin-4-ylamino)- | | |
| | pyridine-3-carbonyl)-amino)-2-methyl- | | |
| | propionic acid | | |
| 57 | 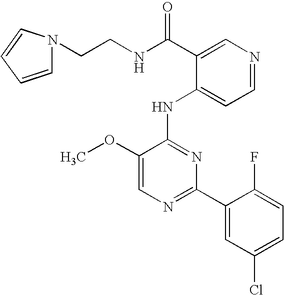 | 467.1, 1.46 | 0.07 |
| | AutoNam Name: | | |
| | 4-[2-(5-Chloro-2-fluoro-phenyl)-5- | | |
| | methoxy-pyrimidin-4-ylamino]-N-(2- | | |
| | pyrrol-1-yl-ethyl)-nicotinamide | | |
| 58 | 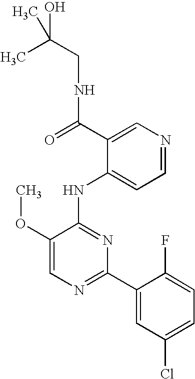 | 446.0, 1.353 | 0.47 |
| | AutoNom Name: | | |
| | 4-[2-(5-Chloro-2-fluoro-phenyl)-5- | | |
| | methoxy-pyrimidin-4-ylamino]-N-(2- | | |
| | hydroxy-2-methyl-propyl)-nicotinamide | | |
| 59 | 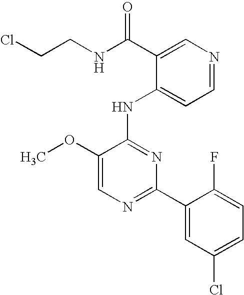 | | |
| | AutoNom Name: | | |
| | N-(2-Chloro-ethyl)-4-[2-(5-chloro-2- | | |
| | fluoro-phenyl)-5-methoxy-pyrimidin- | | |
| | 4-ylamino]-nicotinamide | | |
| 60 | 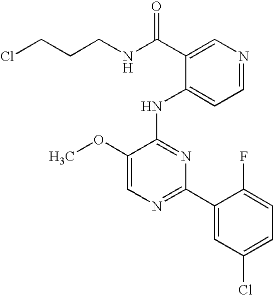 | | |
| | AutoNom Name: | | |
| | 4-(2-(5-Chloro-2-fluoro-phenyl)-5- | | |
| | methoxy-pyrimidin-4-ylamino]-N-(3- | | |
| | chloro-propyl)-nicotinamide | | |
| 61 | 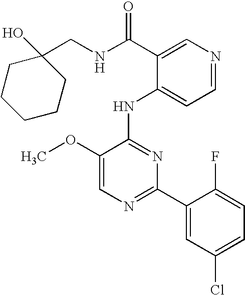 | 486.1, 1.48 | 11.85 |
| | AutoNom Name: | | |
| | 4-[2-(5-Chloro-2-fluoro-phenyl)-5- | | |
| | methoxy-pyrimidin-4-ylamino]-N-(1- | | |
| | hydroxy-cyclohexylmethyl)-nicotinamide | | |
| 62 | 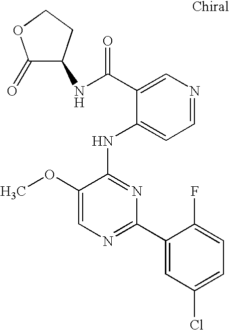 | 458.0, 1.31 | 0.36 |
| | AutoNom Name: | | |
| | 4-[2-(5-Chloro-2-fluoro-phenyl)-5- | | |
| | methoxy-pyrimidin-4-ylamino]-N-(2-oxo- | | |
| | tetrahydro-furan-3-yl)-nicotinamide | | |
| 63 | 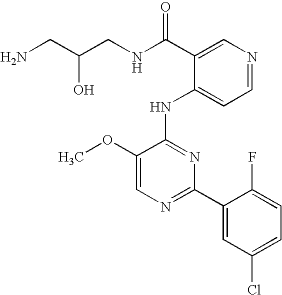 | 447.0, 1.073 | 0.10 |
| | AutoNom Name: | | |
| | N-(3-Amino-2-hydroxy-propyl)-4-[2- | | |
| | (5-chloro-2-fluoro-phenyl)-5-methoxy- | | |
| | pyrimidin-4-ylamino]-nicotinamide | | |
| 64 | 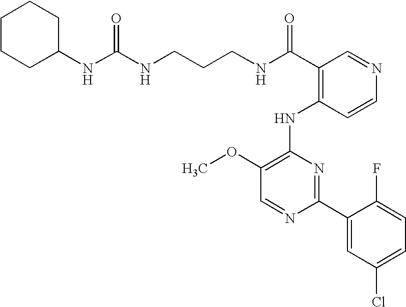 | 555, 1.41 | 0.89 |
| | AutoNom Name: | | |
| | 4-[2-(5-Chloro-2-fluoro-phenyl)-5- | | |
| | methoxy-pyrimidin-4-ylamino]-N-[3-(3- | | |
| | cyclohexyl-ureido)-propyl]-nicotinamide | | |
| 65 | 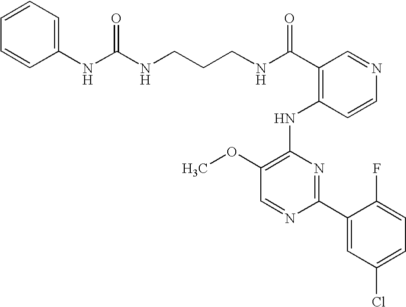 | 609, 1.85 | 0.59 |
| | AutoNom Name: | | |
| | 4-[2-(5-Chloro-2-fluoro-phenyl)-5- | | |
| | methoxy-pyrimidin-4-ylamino]-N-[3-(3- | | |
| | phenyl-ureido)-propyl]-nicotinamide | | |
| 66 | 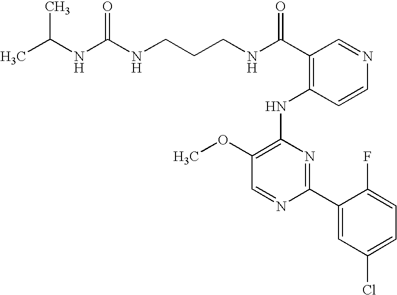 | 515, 1.39 | 0.27 |
| | AutoNom Name: | | |
| | 4-[2-(5-Chloro-2-fluoro-phenyl)-5- | | |
| | methoxy-pyrimidin-4-ylamino]-N-[3-(3- | | |
| | isopropyl-ureido)-propyl]-nicotinamide | | |
| 67 | 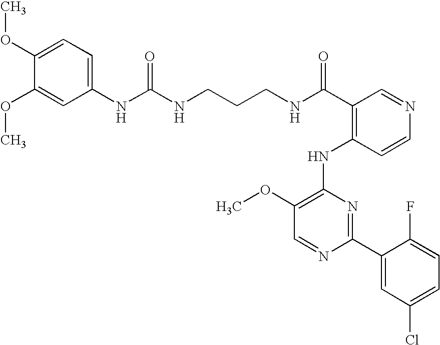 | 609, 1.85 | 0.44 |
| | AutoNom Name: | | |
| | 4-[2-(5-Chloro-2-fluoro-phenyl)-5- | | |
| | methoxy-pyrimidin-4-ylamino]-N-{3-[3- | | |
| | (3,4-dimethoxy-phenyl)-ureido]- | | |
| | propyl}-nicotinamide | | |
| 68 | 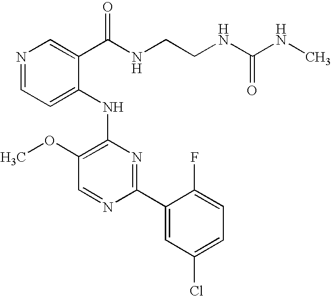 | 473, 1.84 | 1.54 |
| | AutoNom Name: | | |
| | 4-[2-(5-chloro-2-fluoro-phenyl)-5- | | |
| | methoxy-pyrimidin-4-ylamino]-N-[2-(3- | | |
| | methyl-ureido)-ethyl]nicotinamide | | |
| 69 | 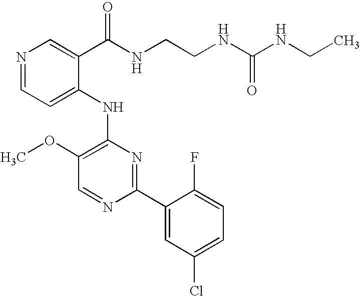 | 487, 1.97 | 0.15 |
| | AutoNom Name: | | |
| | 4-[2-(5-Chloro-2-fluoro-phenyl)-5- | | |
| | methoxy-pyrimidin-4-ylamino]-N-[2-(3- | | |
| | ethyl-ureido)-ethyl]-nicotinamide | | |
| 70 | 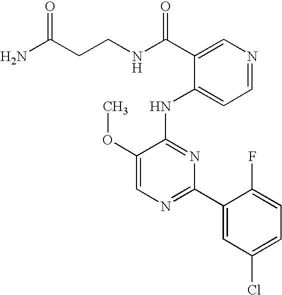 | 445.2, 1.13 | 0.20 |
| | AutoNom Name: | | |
| | N-(2-Carbamoyl-ethyl)-4-[2-(5-choro- | | |
| | 2-fluoro-phenyl)-5-methoxy-pyrimidin- | | |
| | 4-ylamino]-nicotinamide | | |
| 71 | 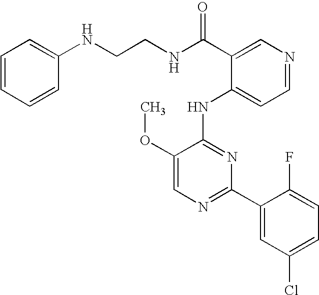 | 493.2, 1.29 | 0.76 |
| | AutoNom Name: | | |
| | 4-[2-(5-Chloro-2-fluoro-phenyl)-5- | | |
| | methoxy-pyrimidin-4-ylamino]-N-(2- | | |
| | phenylamino-ethyl)-nicotinamide | | |
| 72 | 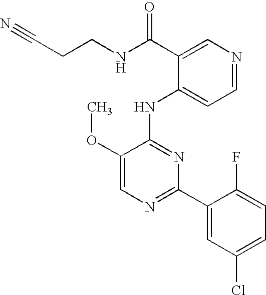 | 427.2, 1.23 | 0.10 |
| | AutoNom Name: | | |
| | 4-[2-(5-Chloro-2-fluoro-phenyl)-5- | | |
| | methoxy-pyrimidin-4-ylamino]-N-(2- | | |
| | cyano-ethyl)-nicotinamide | | |
| 73 | 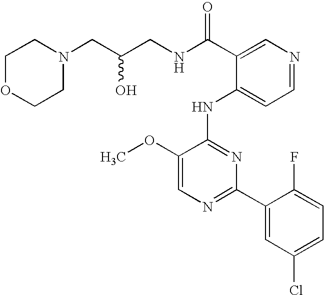 | 517.30, 1.04 | 0.36 |
| | AutoNom Name: | | |
| | 4-[2-(5-Chloro-2-fluoro-phenyl)-5- | | |
| | methoxy-pyrimidin-4-ylamino]-N-(2- | | |
| | hydroxy-3-morpholin-4-yl-propyl)- | | |
| | nicotinamide | | |
| 74 | 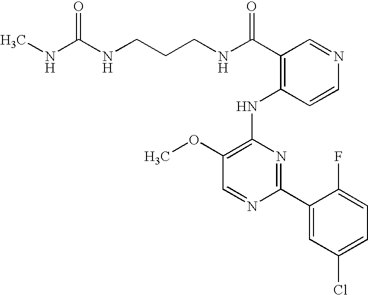 | 487, 1.25 | 0.15 |
| | AutoNom Name: | | |
| | 4-[2-(5-Chloro-2-fluoro-phenyl)-5- | | |
| | methoxy-pyrimidin-4-ylamino]-N-[3-(3- | | |
| | methyl-ureido)-propyl]-nicotinamide | | |
| 75 | 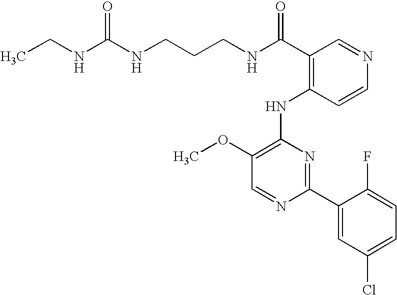 | 501, 1.29 | 0.11 |
| | AutoNom Name: | | |
| | 4-[2-(5-Chloro-2-fluoro-phenyl)-5- | | |
| | methoxy-pyrimidin-4-ylamino[-N-[3-(2- | | |
| | ethyl-ureido)-propyl]-nicotinamide | | |
| 76 | 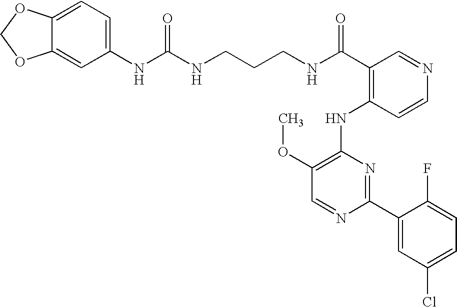 | 593, 1.36 | 0.32 |
| | AutoNom Name: | | |
| | N-[3-(3-Benzo[1,3]dioxol-5-yl-ureido)- | | |
| | propyl]-4-[2-(5-chloro-2-fluoro- | | |
| | phenyl)-5-methoxy-pyrimidin-4-ylamino]- | | |
| | nicotinamide | | |
| 77 | 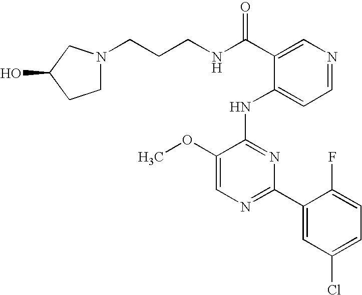 | 501.1, 1.487 | 0.04 |
| | AutoNom Name: | | |
| | 4-[2-(5-Chloro-2-fluoro-phenyl)-5- | | |
| | methoxy-pyrimidin-4-ylamino]-N-[3-(3- | | |
| | hydroxy-pyrrolidin-1-yl)-propyl]- | | |
| | nicotinamide | | |
| 78 | 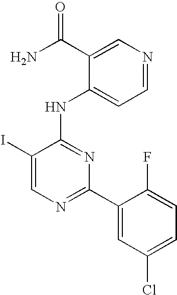 | 470.1, 1.313 | 0.01 |
| | AutoNom Name: | | |
| | 4-[2-(5-Chloro-2-fluoro-phenyl)-5- | | |
| | iodo-pyrimidin-4-ylamino]-nicotinamide | | |
| 79 | 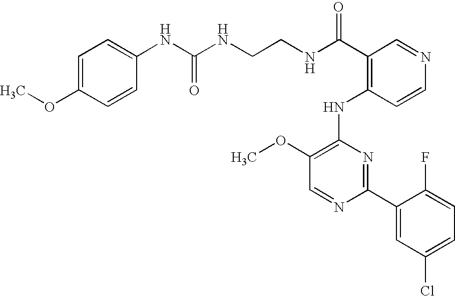 | 565, 1.34 | 0.35 |
| | AutoNom Name: | CI | |
| | 4-[2-(5-Chloro-2-fluoro-phenyl)-5- | | |
| | methoxy-pyrimidin-4-ylamino]-N-{2-[3- | | |
| | (4-methoxy-phenyl)-ureido]-ethyl}- | | |
| | nicotinamide | | |
| 80 | 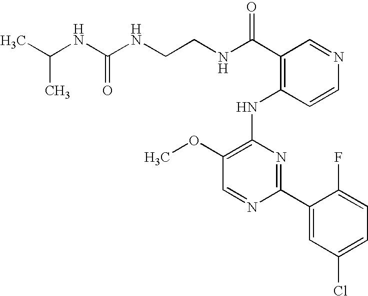 | 501, 1.31 | 0.57 |
| | AutoNom Name: | | |
| | 4-[2-(5-Chloro-2-fluoro-phenyl)-5- | | |
| | methoxy-pyrimidin-4-ylamino]-N-[2-(3- | | |
| | isopropyl-ureido)-ethyl]-nicotinamide | | |
| 81 | 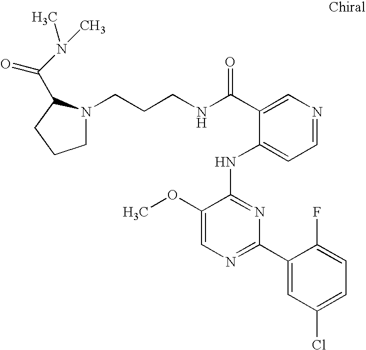 | 556.2, 1.147 | 0.12 |
| | AutoNom Name: | | |
| | 4-[2-(5-Chloro-2-fluoro-phenyl)-5- | | |
| | methoxy-pyrimidin-4-ylamino]-N-[3-(2- | | |
| | dimethylcarbamoyl-pyrrolidin-1-yl)- | | |
| | propyl]-nicotinamide | | |
| 82 | 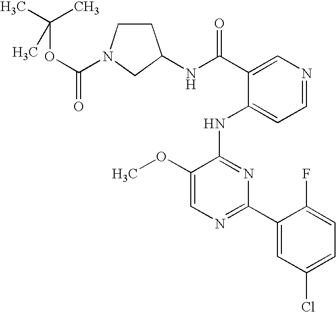 | 543.2, 1.560 | 0.31 |
| | AutoNom Name: | | |
| | 3-((4-[2-(5-Chloro-2-fluoro-phenyl)- | | |
| | 5-methoxy-pyrimidin-4-ylamino]- | | |
| | pyridine-3-carbonyl)-amino)-pyrrolidine- | | |
| | 1-carboxylic acid tert-butyl ester | | |
| 83 | 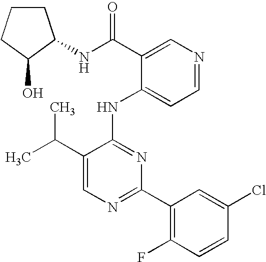 | 470.2, 1.413 | 0.23 |
| | AutoNom Name: | | |
| | 4-[2-(5-Chloro-2-fluoro-phenyl)-5- | | |
| | isopropyl-pyrimidin-4-ylamino)-N-(2- | | |
| | hydroxy-cyclopentyl)-nicotinamide | | |
| 84 | 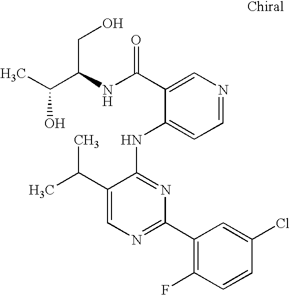 | 474.3, 1.280 | 1.73 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxy-1-hydroxymethyl-propyl)- | | |
| | nicotinamide | | |
| 85 | 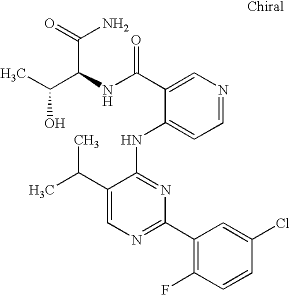 | 487.2, 1.300 | 3.96 |
| | AutoNom Name. | | |
| | N-(1-Carbamoyl-2-hydroxy-propyl)-4- | | |
| | [2-(5-chloro-2-fluoro-phenyl)-5- | | |
| | isopropyl-pyrimidin-4-ylamino]-nicotinamide | | |
| 86 | 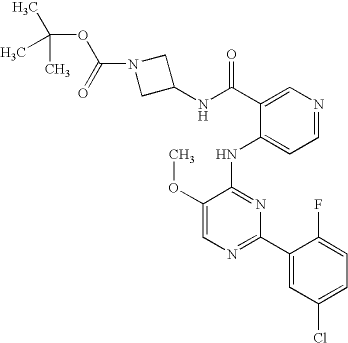 | 529.2, 1.49 | 0.31 |
| | AutoNom Name: | | |
| | 3-({4-[2-(5-Chloro-2-fluoro-phenyl)- | | |
| | 5-methoxy-pyrimidin-4-ylamino]- | | |
| | pyridine-3-carbonyl}-amino)-azetidine- | | |
| | 1-carboxylic acid tert-butyl ester | | |
| 87 | 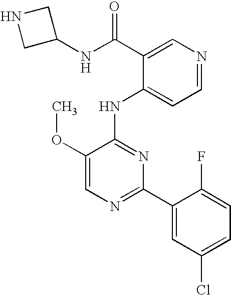 | 429.1, 1.13 | 0.05 |
| | AutoNom Name: | | |
| | N-Azetidin-3-yl-4-[2-(5-chloro-2- | | |
| | fluoro-phenyl)-5-methoxy-pyrimidin-4- | | |
| | ylamino]-nicotinamide | | |
| 88 | 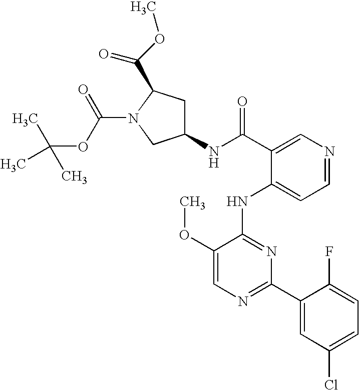 | 601.20, 1.51 | 14.09 |
| | AutoNom Name: | | |
| | 4-({4-[2-(5-Chloro-2-fluoro-phenyl)- | | |
| | 5-methoxy-pyrimidin-4-ylamino]- | | |
| | pyridine-3-carbonyl}-amino)-pyrrolidine- | | |
| | 1,2-dicarboxylic acid 1-tert-butyl | | |
| | ester 2-methyl ester | | |
| 89 | 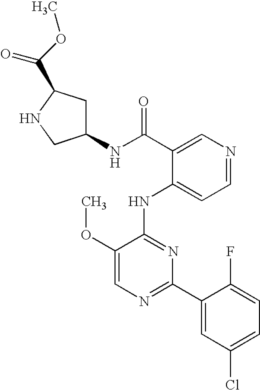 | 502.2, 1.18 | 0.19 |
| | AutoNom Name: | | |
| | 4-({4-[2-(5-Chloro-2-fluoro-phenyl)- | | |
| | 5-methoxy-pyrimidin-4-ylamino]- | | |
| | pyridine-3-carbonyl}-amino)-pyrrolidine- | | |
| | 2-carboxylic acid methyl ester | | |
| 90 | 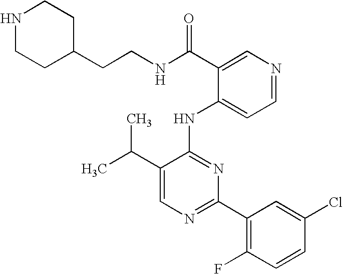 | | |
| | AutoNom Name: | | |
| | 4-[2-(5-Chloro-2-fluoro-phenyl)-5- | | |
| | isopropyl-pyrimidin-4-ylamino]-N-(2- | | |
| | piperidin-4-yl-ethyl)-nicotinamide | | |
| 91 | 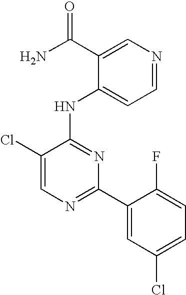 | 378.1, 1.327 | 0.20 |
| | AutoNom Name: | | |
| | 4-[5-Chloro-2-(5-chloro-2-fluoro- | | |
| | phenyl)-pyrimidin-4-ylamino]-nicotinamide | | |
| 92 | 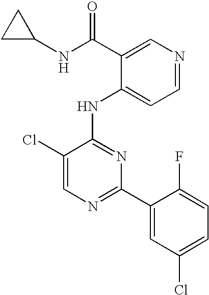 | 418.0, 1.420 | 0.13 |
| | AutoNom Name: | | |
| | 4-[5-Chloro-2-(5-chloro-2-fluoro- | | |
| | phenyl)-pyrimidin-4-ylamino]-N- | | |
| | cyclopropyl-nicotinamide | | |
| 93 | 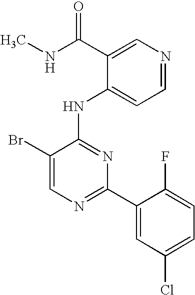 | 437.9, 1.447 | 0.03 |
| | AutoNom Name: | | |
| | 4-[5-Bromo-2-(5-chloro-2-fluoro- | | |
| | phenyl)-pyrimidin-4-ylamino]-N-methyl- | | |
| | nicotinamide | | |
| 94 | 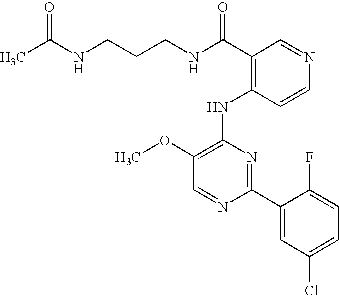 | 472, 1.23 | 1.34 |
| | AutoNom Name: | | |
| | N-(3-Acetylamino-propyl)-4-[2-(5- | | |
| | chloro-2-fluoro-phenyl)-5-methoxy- | | |
| | pyrimidin-4-ylamino]-nicotinamide | | |
| 95 | 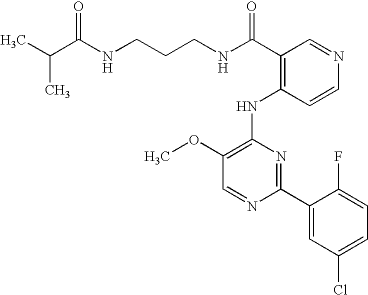 | 500, 1.33 | 0.17 |
| | AutoNom Name: | | |
| | 4-[2-(5-Chloro-2-fluoro-phenyl)-5- | | |
| | methoxy-pyrimidin-4-ylamino]-N-(3- | | |
| | isobutylylamino-propyl)-nicotinamide | | |
| 96 | 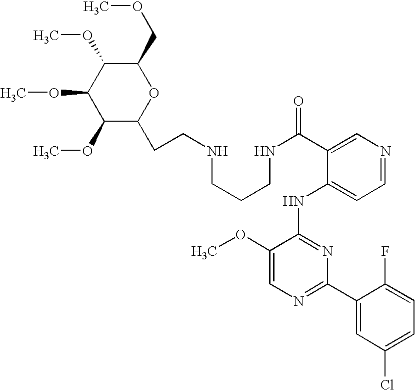 | 677.5, 1.18 | 0.07 |
| | AutoNom Name: | | |
| | 4-[2-(5-Chloro-2-fluoro-phenyl)-5- | | |
| | methoxy-pyrimidin-4-ylamino]-N-(3-[2- | | |
| | (3,4,5-trimethoxy-6-methoxymethyl- | | |
| | tetrahydro-pyran-2-yl)-ethylamino]- | | |
| | propyl)-nicotinamide | | |
| 97 | 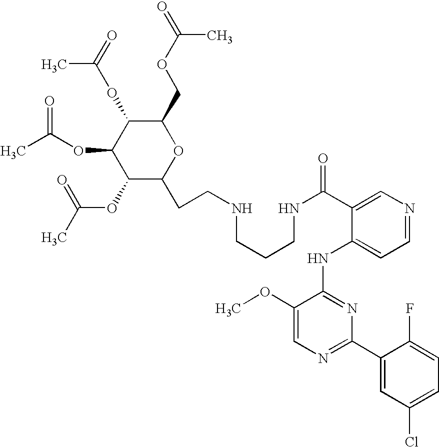 | 789.5, 1.287 | 0.09 |
| | AutoNom Name: | | |
| | Acetic acid 3,4,5-triacetoxy-6-{2-[3- | | |
| | ({4-[2-(5-chloro-2-fluoro-phenyl)- | | |
| | 5-methoxy-pyrimidin-4-ylamino]- | | |
| | pyridine-3-carbonyl}-amino)-propylamino]- | | |
| | ethyl}-tetrahydro-pyran-2-ylmethyl | | |
| | ester | | |
| 98 | 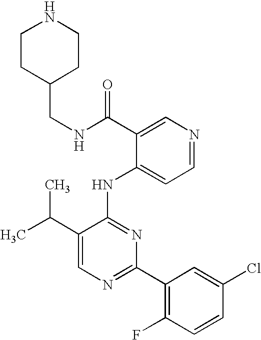 | | |
| | AutoNom Name: | | |
| | 4-[2-(5-Chloro-2-fluoro-phenyl)-5- | | |
| | isopropyl-pyrimidin-4-ylamino]-N- | | |
| | piperidin-4-ylmethyl-nicotinamide | | |
| 99 | 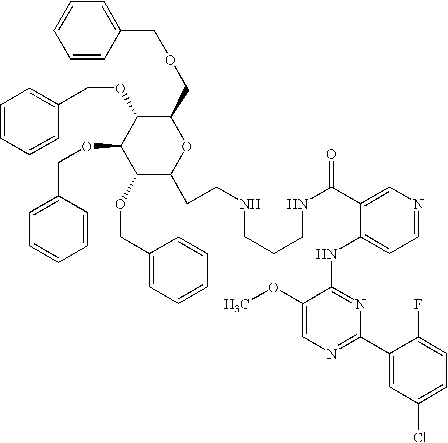 | 981.7, 1.993 | 0.61 |
| | AutoNom Name: | | |
| | 4-[2-(5-Chloro-2-fluoro-phenyl)-5- | | |
| | methoxy-pyrimidin-4-ylamino]-N-{3-[2- | | |
| | (3,4,5-tris-benzyloxy-6-benzyloxy- | | |
| | methyl-tetrahydro-pyran-2-yl)-ethylamino]- | | |
| | propyl}-nicotinamide | | |
| 100 | 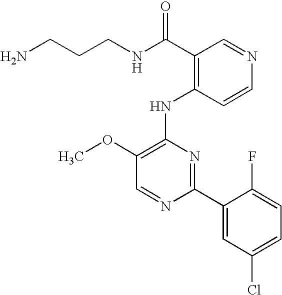 | 430, 1.06 | 0.03 |
| | AutoNom Name: | | |
| | N-(3-Amino-propyl)-4-[2-(5-chloro-2- | | |
| | fluoro-phenyl)-5-methoxy-pyrimidin- | | |
| | 4-ylamino]-nicotinamide | | |
| 101 | 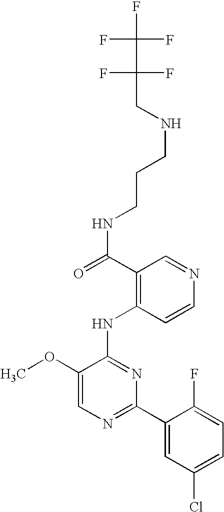 | 563.0, 1.26 | 0.14 |
| | AutoNom Name: | | |
| | 4-[2-(5-Chloro-2-fluoro-phenyl)-5- | | |
| | methoxy-pyrimidin-4-ylamino]-N-[3-(2, | | |
| | 2,3,3,3-pentafluoro-propylamino)- | | |
| | propyl]-nicotinamide | | |
| 102 | 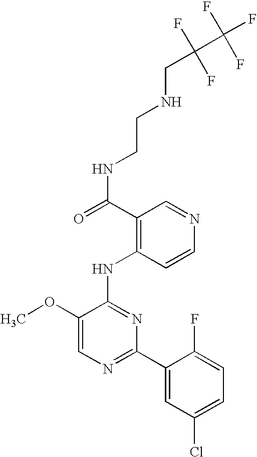 | 549.1, 1.26 | 0.07 |
| | AutoNom Name: | | |
| | 4-[2-(5-Chloro-2-fluoro-phenyl)-5- | | |
| | methoxy-pyrimidin-4-ylamino]-N-[2-(2, | | |
| | 2,3,3,3-pentafluoro-propylamino)- | | |
| | ethyl]-nicotinamide | | |
| 103 | 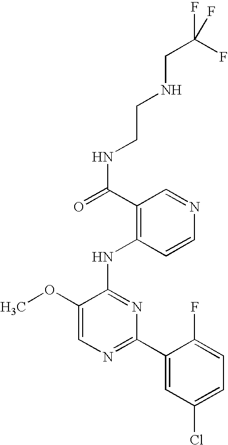 | 499.2, 1.17 | 0.13 |
| | AutoNom Name: | | |
| | 4-[2-(5-Chloro-2-fluoro-phenyl)-5- | | |
| | methoxy-pyrimidin-4-ylamino]-N-[2-(2, | | |
| | 2,2-trifluoro-ethylamino)-ethyl]- | | |
| | nicotinamide | | |
| 104 | 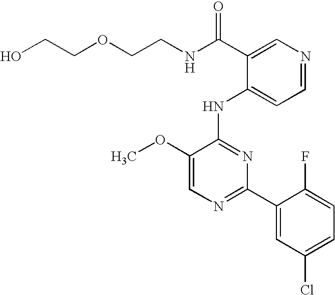 | 462.1, 1.19 | 0.31 |
| | AutoNom Name: | | |
| | 4-[2-(5-Chloro-2-fluoro-phenyl)-5- | | |
| | methoxy-pyrimidin-4-ylamino]-N-[2-(2- | | |
| | hydroxy-ethoxy)-ethyl]-nicotinamide | | |
| 105 | 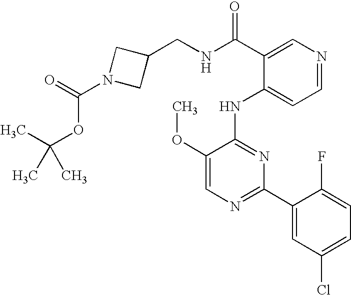 | 543.2, 1.53 | 0.24 |
| | AutoNom Name: | | |
| | 3-[({4-[2-(5-Chloro-2-fluoro-phenyl)- | | |
| | 5-methoxy-pyrimidin-4-ylamino]- | | |
| | pyridine-3-carbonyl}-amino)-methyl- | | |
| | azetidine-1-carboxylic acid tert-butyl | | |
| | ester | | |
| 106 | 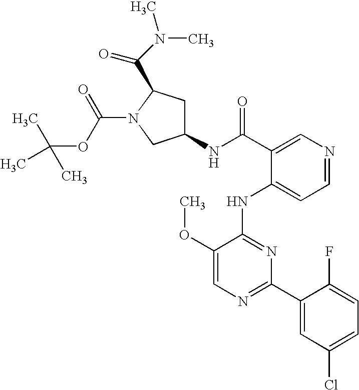 | 614.3, 1.47 | 4.81 |
| | AutoNom Name: | | |
| | 4-({4-[2-(5-Chloro-2-fluoro-phenyl)- | | |
| | 5-methoxy-pyrimidin-4-ylamino]- | | |
| | pyridine-3-carbonyl}-amino)-2-dimethyl | | |
| | carbamoyl-pyrrolidine-1-carboxylic | | |
| | acid tert-butyl ester | | |
| 107 | 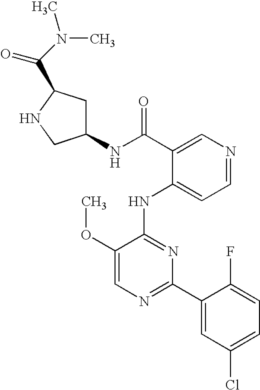 | 514.10, 1.11 | 0.09 |
| | AutoNom Name: | | |
| | 4-[2-(5-Chloro-2-fluoro-phenyl)-5- | | |
| | methoxy-pyrimidin-4-ylamino]-N-(5- | | |
| | dimethylcarbamoyl-pyrrolidin-3-yl)- | | |
| | nicotinamide | | |
| 108 | 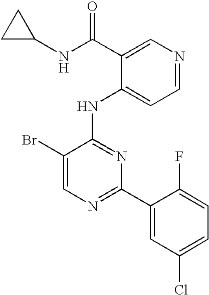 | 464.2, 1.273 | 2.50 |
| | AutoNom Name: | | |
| | 4-[5-Bromo-2-(5-chloro-2-fluoro-phenyl)- | | |
| | pyrimidin-4-ylamino]-N-cyclopropyl- | | |
| | nicotinamide | | |
| 109 | 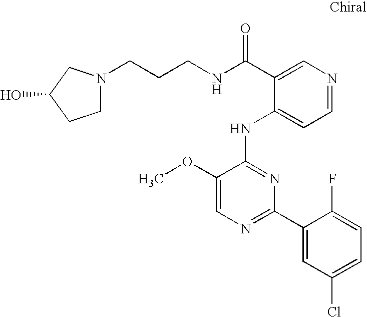 | 501.3, 1.087 | 0.33 |
| | AutoNom Name: | | |
| | 4-[2-(5-Chloro-2-fluoro-phenyl)-5- | | |
| | methoxy-pyrimidin-4-ylamino]-N-[3-(3- | | |
| | hydroxy-pyrolidin-1-yl)-propyl]- | | |
| | nicotinamide | | |
| 110 | 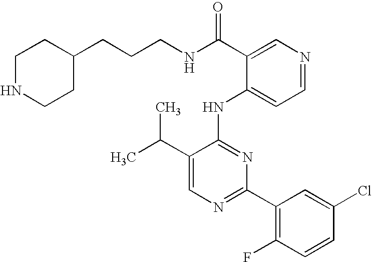 | | |
| | AutoNom Name: | | |
| | 4-[2-(5-Chloro-2-fluoro-phenyl)-5- | | |
| | isopropyl-pyrimidin-4-ylamino]-N-(3- | | |
| | piperidin-4-yl-propyl)-nicotinamide | | |
| 111 | 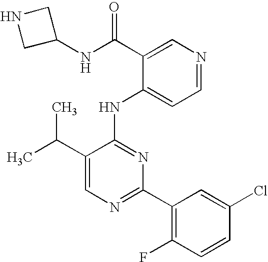 | 441.4,1.153 | 0.02 |
| | AutoNom Name: | | |
| | N-Azetidin-3-yl-4-[2-(5-chloro-2- | | |
| | fluoro-phenyl)-5-isopropyl-pyrimidin- | | |
| | 4-ylamino]-nicotinamide | | |
| 112 | 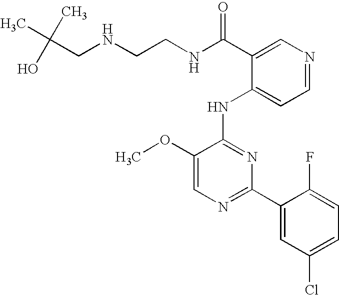 | 489.2, 1.100 | 0.09 |
| | AutoNom Name; | | |
| | 4-[2-(5-Chloro-2-fluoro-phenyl)-5- | | |
| | methoxy-pyrimidin-4-ylamino]-N-[2-(2- | | |
| | hydroxy-2-methyl-propylamino)-ethyl]- | | |
| | nicotinamide | | |
| 113 | 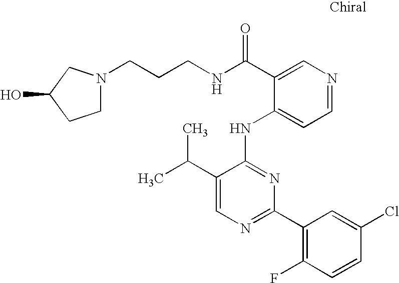 | 513.1, 1.257 | 0.04 |
| | AutoNom Name: | | |
| | 4-[2-(5-Chloro-2-fluoro-phenyl)-5- | | |
| | isopropyl-pyrimidin-4-ylamino]-N-[3- | | |
| | (3-hydroxy-pyrrolidin-1-yl)-propyl]- | | |
| | nicotinamide | | |
| 114 | 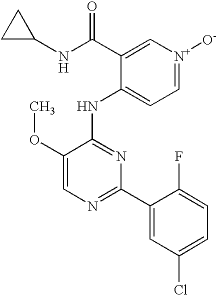 | 430.1, 1.34 | 3.08 |
| | AutoNom Name: | | |
| | 4-[2-(5-Chloro-2-fluoro-phenyl)-5- | | |
| | methoxy-pyrimidin-4-ylamino]-N- | | |
| | cyclopropyl-1-oxy-nicotinamide | | |
| 115 | 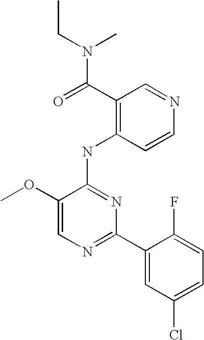 | | |
| | AutoNom Name: | | |
| | 4-[2-(5-Chloro-2-fluoro-phenyl)-5- | | |
| | methoxy-pyrimidin-4-ylamino]-N-ethyl- | | |
| | N-methyl-nicotinamide | | |
| 116 | 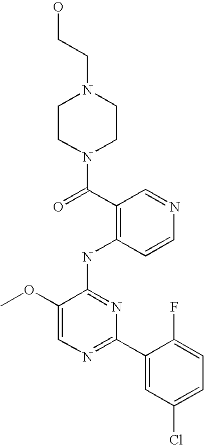 | 487.17, 2.25 | 7.37 |
| | AutoNom Name: | | |
| | {4-[2-(5-Chloro-2-fluoro-phenyl)5- | | |
| | methoxy-pyrimidin-4-ylamino]pyridin- | | |
| | 3-yl}-[4-(2-hydroxy-ethyl)-piperazin- | | |
| | 1-yl]-methanone | | |
| 117 | 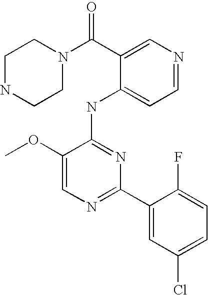 | 443.1, 1.22 | 6.78 |
| | AutoNom Name: | | |
| | {4-[2-(5-Chloro-2-fluoro-phenyl)-5- | | |
| | methoxy-pyrimidin-4-ylamino]-pyridin- | | |
| | 3-yl}-piperazin-1-yl-methanone | | |
| 118 | 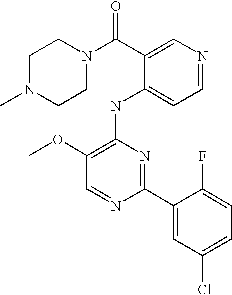 | 457.0, 1.15 | 6.57 |
| | AutoNom Name: | | |
| | {4-[2-(5-Chloro-2-fluoro-phenyl)-5- | | |
| | methoxy-pyrimidin-4-ylamino]-pyridin- | | |
| | 3-yl}-(4-methyl-piperazin-1-yl)- | | |
| | methanone | | |
| 119 | 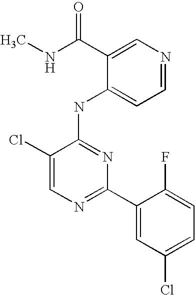 | 391.9, 1.33 | 0.155 |
| | AutoNom Name: | | |
| | 4-[5-Chloro-2-(5-chloro-2-fluoro- | | |
| | phenyl)-pyrimidin-4-ylamino]-N-methyl- | | |
| | nicotinamide | | |
| 120 | 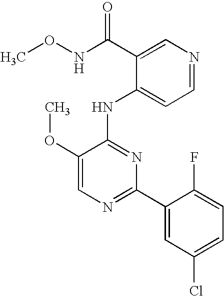 | 404.2, 1.29 | 0.19 |
| | AutoNom Name: | | |
| | 4-[2-(5-Chloro-2-fluoro-phenyl)-5- | | |
| | methoxy-pyrimidin-4-ylamino]-N-methoxy- | | |
| | nicotinamide | | |
| 121 | 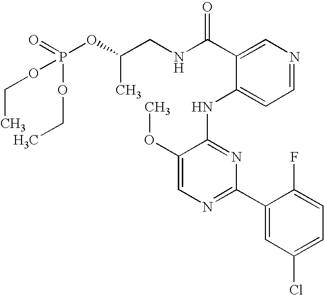 | 568.3, 1.48 | 5.766 |
| | AutoNom Name: | | |
| | Phosphoric acid 2-({4-[2-(5-chloro- | | |
| | 2-fluoro-phenyl)-5-methoxy-pyrimidin- | | |
| | 4-ylamino]-pyridine-3-carbonyl}- | | |
| | amino)-1-methyl-ethyl ester diethyl | | |
| | ester | | |
| 122 | 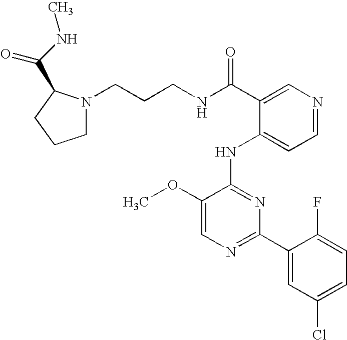 | 542.3, 1.05 | 0.053 |
| | AutoNom Name: | | |
| | 4-[2-(5-Chloro-2-fluoro-phenyl)-5- | | |
| | methoxy-pyrimidin-4-ylamino]-N-[3-(2- | | |
| | methyl carbamoyl-pyrrolidin-1-yl)- | | |
| | propyl]-nicotinamide | | |
| 123 | 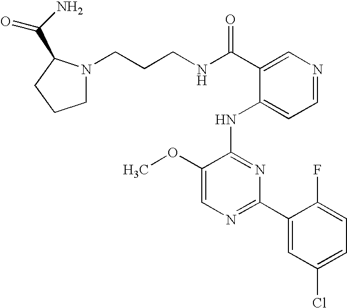 | 528.2, 1.06 | 0.023 |
| | AutoNom Name: | | |
| | N-[3-(2-Carbamoyl-pyrrolidin-1-yl)- | | |
| | propyl]-4-[2-(5-chloro-2-fluoro- | | |
| | phenyl)-5-methoxy-pyrimidin-4-ylamino]- | | |
| | nicotinamide | | |
| 124 | 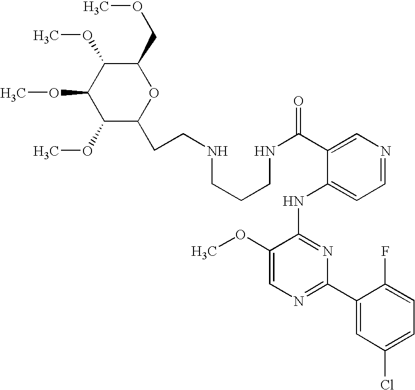 | 677.5, 1.24 | 0.042 |
| | AutoNom Name: | | |
| | 4-[2-(5-Chloro-2-fluoro-phenyl)-5- | | |
| | methoxy-pyrimidin-4-ylamino]-N-{3-[2- | | |
| | (3,4,5-trimethoxy-6-methoxymethyl- | | |
| | tetrahydro-pyran-2-yl)-ethylamino]- | | |
| | propyl}-nicotinamide | | |
| 125 | 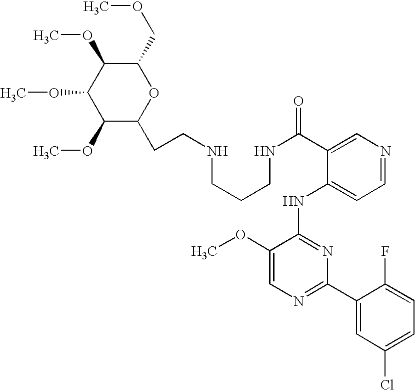 | 677.1, 1.25 | 0.032 |
| | AutoNom Name: | | |
| | 4-[2-(5-Chloro-2-fluoro-phenyl)-5- | | |
| | methoxy-pyrimidin-4-ylamino]-N-{3-[2- | | |
| | (3,4,5-trimethoxy-6-methoxymethyl- | | |
| | tetrahydro-pyran-2-yl)-ethylamino]- | | |
| | propyl}-nicotinamide | | |
| 126 | 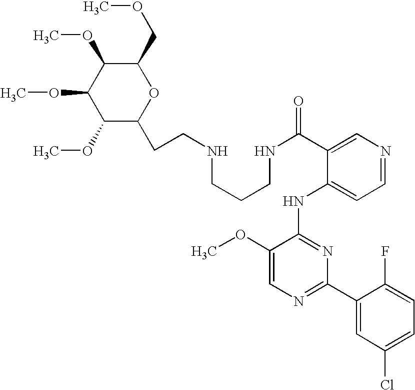 | 677.6, 1.21 | 0.05 |
| | AutoNom Name: | | |
| | 4-[2-(5-Chloro-2-fluoro-phenyl)-5- | | |
| | methoxy-pyrimidin-4-ylamino]-N-{3-[2- | | |
| | 3,4,5-trimethoxy-6-methoxymethyl- | | |
| | tetrahydro-pyran-2-yl)-ethylamino]- | | |
| | propyl}-nicotinamide | | |
| 127 | 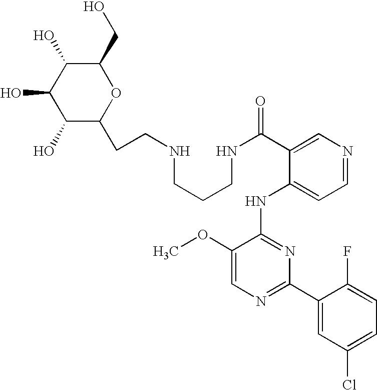 | 621.4, 1.05 | 0.063 |
| | AutoNom Name: | | |
| | 4-[2-(5-Chloro-2-fluoro-phenyl)-5- | | |
| | methoxy-pyrimidin-4-ylamino]-N-{3-[2- | | |
| | (3,4,5-trihydroxy-6-hydroxymethyl- | | |
| | tetrahydro-pyran-2-yl)-ethylamino]- | | |
| | propyl}-nicotinamide | | |
| 128 | 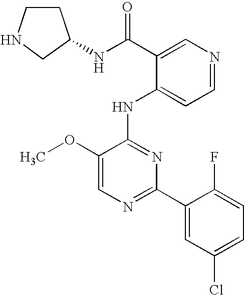 | 443.1, 1.11 | 0.008 |
| | AutoNom Name: | | |
| | 4-[2-(5-Chloro-2-fluoro-phenyl)-5- | | |
| | methoxy-pyrimidin-4-ylamino]-N- | | |
| | pyrrolidin-3-yl-nicotinamide | | |
| 129 | 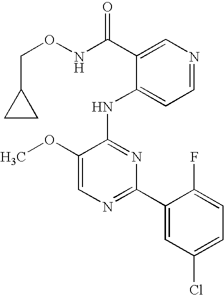 | 444.3, 1.44 | 0.059 |
| | AutoNom Name: | | |
| | 4-[2-(5-Chloro-2-fluoro-phenyl)-5- | | |
| | methoxy-pyrimidin-4-ylamino]-N- | | |
| | cyclopropylmethoxy-nicotinamide | | |
| 130 | 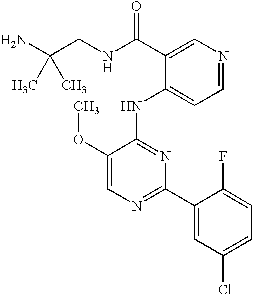 | 445.5, 1.28 | 0.038 |
| | AutoNom Name: | | |
| | N-(2-Amino-2-methyl-propyl)-4-[2-(5- | | |
| | chloro-2-fluoro-phenyl)-5-methoxy- | | |
| | pyrimidin-4-ylamino]-nicotioamide | | |
| 131 | 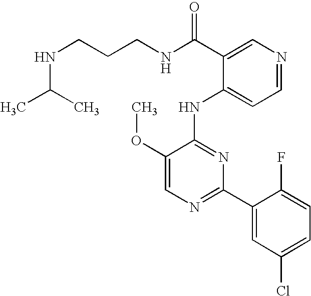 | 473.4, 1.15 | 0.021 |
| | AutoNom Name: | | |
| | 4-[2-(5-Chloro-2-fluoro-phenyl)-5- | | |
| | methoxy-pyrimidin-4-ylamino]-N-(3- | | |
| | isoopropyl-amino-propyl)-nicotinamide | | |
| 132 | 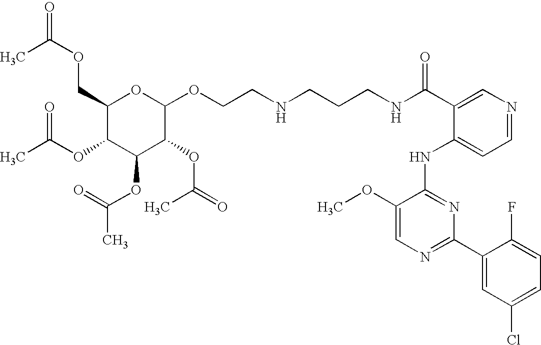 | 805.5, 1.30 | 0.032 |
| | AutoNom Name: | | |
| | Acetic acid 3,4,5-triacetoxy-6-{2-[3- | | |
| | ({4-[2-(5-chloro-2-fluoro-phenyl)- | | |
| | 5-methoxy-pyrimidin-4-ylamino]- | | |
| | pyridine-3-carbonyl)-amino)-propylamino]- | | |
| | ethoxy}-tetrahydro-pyran-2- | | |
| | ylmethyl ester | | |
| 133 | 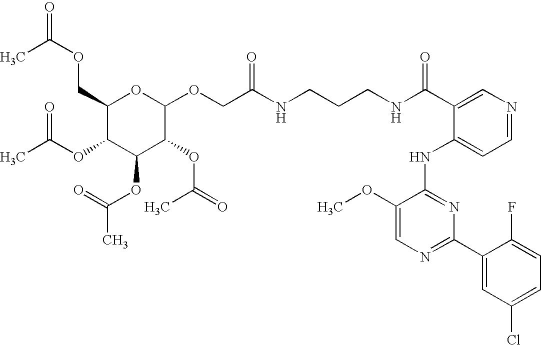 | 819.5, 1.43 | 0.327 |
| | AutoNom Name: | | |
| | Acetic acid 3,4,5-triacetoxy-6-{[3- | | |
| | ({4-[2-(5-chloro-2-fluro-phenyl)-5- | | |
| | methoxy-pyrimidin-4-ylamino]-pyridine- | | |
| | 3-carbonyl{-amino)-propylcarbamoyl]- | | |
| | methoxy}tetrahydro-pyran-2-yl- | | |
| | methyl ester | | |
| 134 | 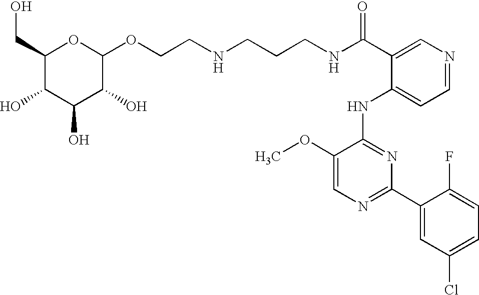 | 637.4, 1.05 | 0.012 |
| | AutoNam Name: | | |
| | 4-[2-(5-Chloro-2-fluoro-phenyl)-5- | | |
| | methoxy-pyrimidin-4-ylamino]-N-{3-[2- | | |
| | (3,4,5-trihydroxy-6-hydroxymethyl- | | |
| | tetrahydro-pyran-2-yloxy)-ethylamino]- | | |
| | propyl}-nicotinamide | | |
| 135 | 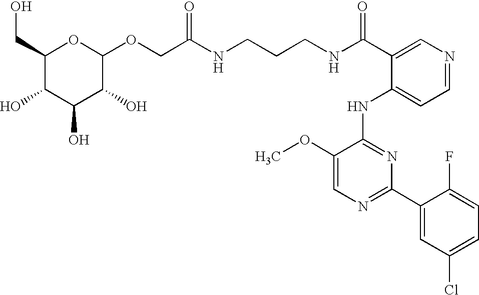 | 651.4, 1.11 | 0.06 |
| | AutoNom Name: | | |
| | 4-[2-(5-Chloro-2-fluoro-phenyl)-5- | | |
| | methoxy-pyrimidin-4-ylamino]-N-{3[2- | | |
| | (3,4,5-trihydroxy-6-hydroxymethyl- | | |
| | tetrahydro-pyran-2-yloxy)-acetylamino]- | | |
| | propyl)-nicotinamide | | |
| 136 | 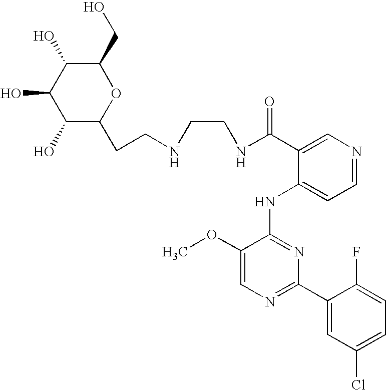 | 607.4, 1.03 | 0.007 |
| | AutoNom Name: | | |
| | 4-[2-(5-Chloro-2-fluoro-phenyl)-5- | | |
| | methoxy-pyrimidin-4-ylamino]-N-{2-[2- | | |
| | (3,4,5-trihydroxy-6-hydroxymethyl- | | |
| | tetrahydro-pyran-2-yl)-ethylamino]- | | |
| | ethyl}-nicotinamide | | |
| 137 | 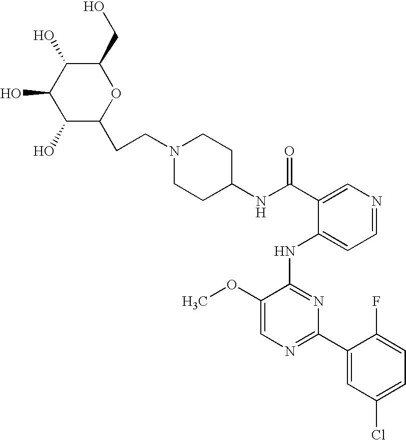 | 647.4, 1.07 | 0.018 |
| | AutoNom Name: | | |
| | 4-[2-(5-Chloro-2-fluoro-phenyl)-5- | | |
| | methoxy-pyrimidin-4-ylamino]-N-{1-[2- | | |
| | (3,4,5-trihydroxy-6-hydroxymethyl- | | |
| | tetrahydro-pyran-2-yl)-ethyl]-piperidin- | | |
| | 4-yl}-nicotinamide | | |
| 138 | 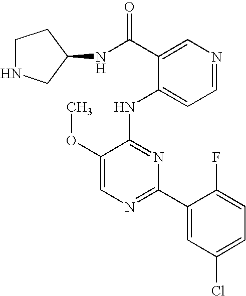 | 443.4,1.10 | 0.024 |
| | AutoNom Nome: | | |
| | 4-[2-(5-Chloro-2-fluoro-phenyl)-5- | | |
| | methoxy-pyrimidin-4-ylamino]-N- | | |
| | pyrrolidin-3-yl-nicotinamide | | |
| 139 | 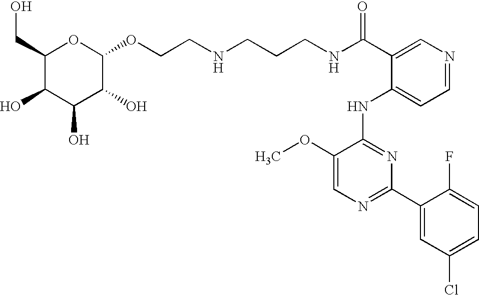 | 637.3, 1.03 | 0.007 |
| | AutoNom Name: | | |
| | 4-[2-(5-Chloro-2-fluoro-phenyl)-5- | | |
| | methoxy-pyrimidin-4-ylamino]-N-{3-[2- | | |
| | (3,4,5-trihydroxy-6-hydroxymethyl- | | |
| | tetrahydro-pyran-2-yloxy)-ethylamino]- | | |
| | propyl}-nicotinamide | | |
| 140 | 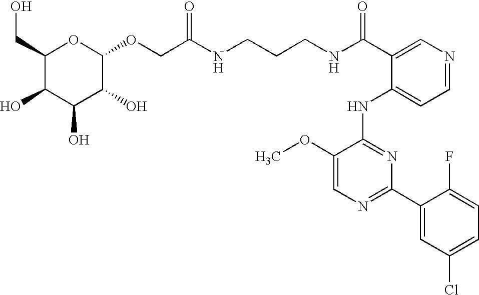 | 651.4, 1.07 | 0.044 |
| | AutoNom Name: | | |
| | 4-[2-(5-Chloro-2-fluoro-phenyl)-5- | | |
| | methoxy-pyrimidin-4-ylamino]-N-{3-[2- | | |
| | (3,4,5-trihydroxy-6-hydroxymethyl- | | |
| | tetrahydro-pyran-2-yloxy)-acetylamino]- | | |
| | propyl}-nicotinamide | | |
| 141 | 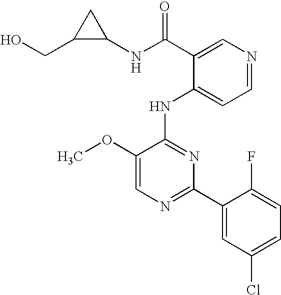 | 444.3, 1.19 | 0.038 |
| | AutoNom Name: | | |
| | 4-[2-(5-Chloro-2-fluoro-phenyl)-5- | | |
| | methoxy-pyrimidin-4-ylamino]-N-(2- | | |
| | hydroxymethyl-cyclopropyl)-nicotinamide | | |
| 142 | 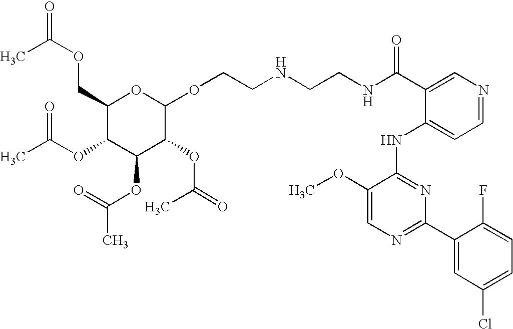 | 396.3, 1.29 | 0.01 |
| | AutoNom Name: | | |
| | Acetic acid 3,4,5-triacetoxy-6-(2-[2- | | |
| | ({4-[2-(5-chloro-2-fluoro-phenyl)- | | |
| | 5-methoxy-pyrimidin-4-ylamino]- | | |
| | pyridine-3-carbonyl]-amino)-ethylamino]- | | |
| | ethoxy}tetrahydro-pyran-2-ylmethyl | | |
| | ester | | |
| 143 | 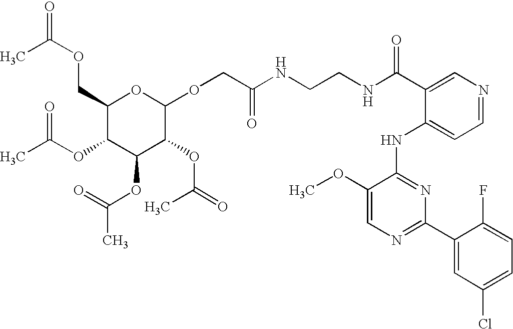 | 805.5, 1.37 | 0.183 |
| | AutoNom Name: | | |
| | Acetic acid 3,4,5-triacetoxy-6-{[2- | | |
| | ({4-[2-(5-chloro-2-fluoro-phenyl)-5- | | |
| | methoxy-pyrimidin-4-ylamino]-pyridine- | | |
| | 3-carbonyl}-amino)-ethylcarbamoyl]- | | |
| | methoxy}-tetrahydro-pyran-2- | | |
| | ylmethyl ester | | |
| 144 | 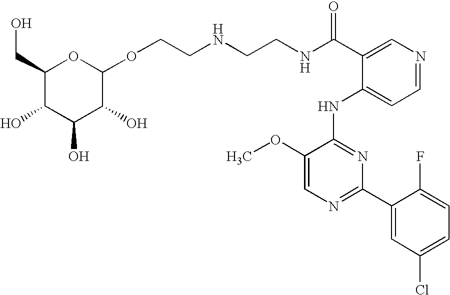 | 623.4, 1.02 | 0.01 |
| | AutoNom Name: | | |
| | 4-[2-(5-chloro-2-fluoro-phenyl)-5- | | |
| | methoxy-pyrimidin-4-ylamino]-N-{2-[2- | | |
| | (3,4,5-trihydroxy-6-hydroxymethyl- | | |
| | tetrahydro-pyran-2-yloxy)-ethylamino]- | | |
| | ethyl)-nicotinamide | | |
| 145 | 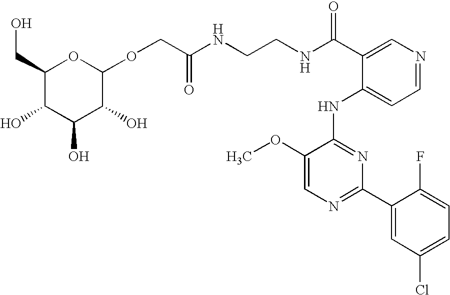 | 637.4, 1.07 | 0.053 |
| | AutoNom Name: | | |
| | 4-[2-(5-Chloro-2-fluoro-phenyl)-5- | | |
| | methoxy-pyrimidin-4-ylamino]-N-{2-[2- | | |
| | (3,4,5-trihydroxy-6-hydroxymethyl- | | |
| | tetrahydro-pyran-2-yloxy)-acetylamino]- | | |
| | ethyl)-nicotinamide | | |
| 146 | 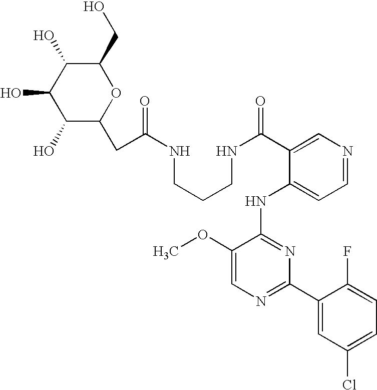 | 635.4, 1.11 | 0.102 |
| | AutoNom Name: | | |
| | 4-[2-(5-Chloro-2-fluoro-phenyl)-5- | | |
| | ethoxy-pyrimidin-4-ylamino]-N-{3-[2- | | |
| | (3,4,5-trihydroxy-6-hydroxymethyl- | | |
| | tetrahydro-pyran-2-yl)-acetylamino]- | | |
| | propyl}-nicotinamide | | |
| 147 | 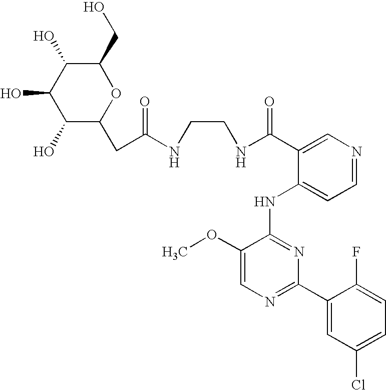 | 621.4, 1.10 | 0.125 |
| | AutoNom Name: | | |
| | 4-[2-(5-Chloro-2-fluoro-phenyl)-5- | | |
| | methoxy-pyrimidin-4-ylamino]-N-{2-[2- | | |
| | (3,4,5-trihydroxy-6-hydroxymethyl- | | |
| | tetrahydro-pyran-2-yl)-acatylamino]- | | |
| | ethyl}-nicotinamide | | |
| 148 | 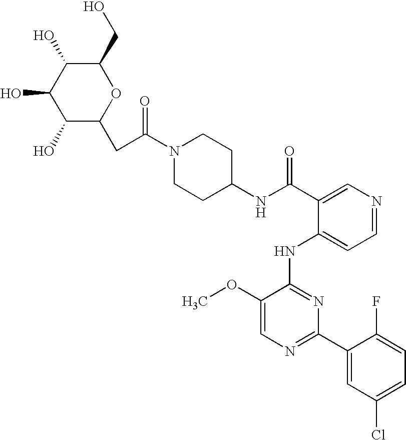 | 661.4, 1.16 | 0.207 |
| | AutoNom Name: | | |
| | 4-[2-(5-Chloro-2-fluoro-phenyl)-5- | | |
| | methoxy-pyrimidin-4-ylamino]-N-{1-[2- | | |
| | (3,4,5-trihydroxy-6-hydroxymethyl- | | |
| | tetrahydro-pyran-2-yl)-acetyl]-piperidin- | | |
| | 4-yl}-nicotinamide | | |
| 149 | 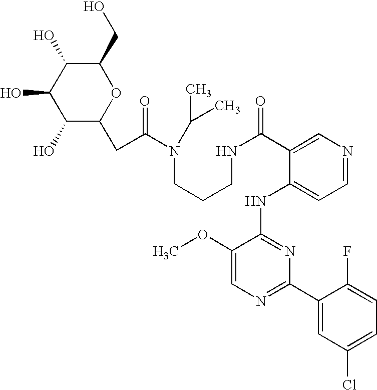 | 677.5, 1.19 | 0.047 |
| | AutoNom Name: | | |
| | 4-[2-(5-Chloro-2-fluoro-phenyl)-5- | | |
| | methoxy-pyrimidin-4-ylamino]-N-(3- | | |
| | [isopropyl-[2-(3,4,5-trihydroxy-6- | | |
| | hydroxymethyl-tetrahydro-pyran-2-yl)- | | |
| | acetyl]-amino]-propyl)-nicotinamide | | |
| 150 | 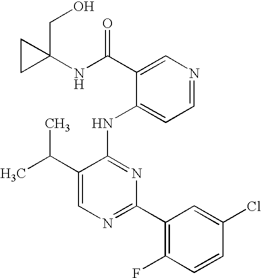 | 456.3, 1.35 | 0.01 |
| | AutoNom Name: | | |
| | 4-[2-(5-Chloro-2-fluoro-phenyl)-5- | | |
| | isopropyl-pyrimidin-4-ylamino]-N-(1- | | |
| | hydroxymethyl-cyclopropyl)-nicotinamide | | |
| 151 | 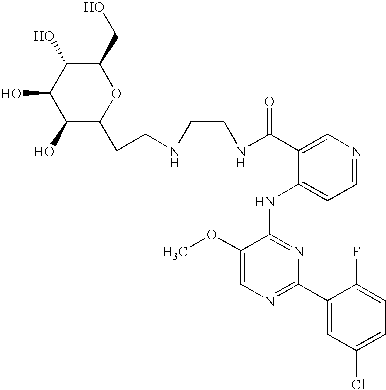 | 607.3, 1.01 | 0.004 |
| | AutoNom Name: | | |
| | 4-[2-(5-Chloro-2-fluoro-phenyl)-5- | | |
| | methoxy-pyrimidin-4-ylamino]-N-{2-[2- | | |
| | (3,4,5-trihydroxy-4-hydroxymethyl- | | |
| | tetrahydro-pyran-2-yl)-ethylamino]- | | |
| | ethyl}-nicotinamide | | |
| 152 | 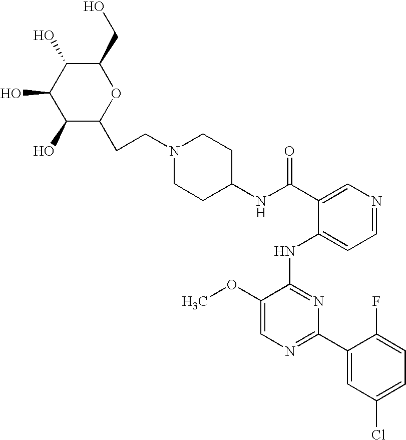 | 647.4, 1.06 | 0.028 |
| | AutoNom Name: | | |
| | 4-[2-(5-Chloro-2-fluoro-phenyl)-5- | | |
| | methoxy-pyrimidin-4-ylamino]-N-{1-[2- | | |
| | (3,4,5-trihydroxy-6-hydroxymethyl- | | |
| | tetrahydro-pyran-2-yl)-ethyl]-piperidin- | | |
| | 4-yl}-nicotinamide | | |
| 153 | 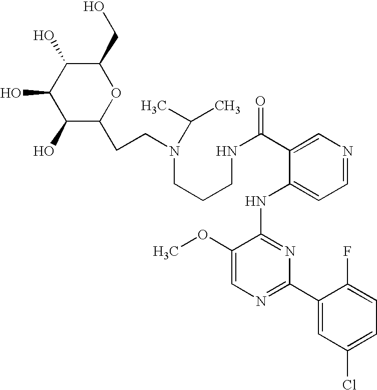 | 663.4, 1.06 | 0.019 |
| | AutoNom Name: | | |
| | 4-[2-(5-Chloro-2-fluoro-phenyl)-5- | | |
| | methoxy-pyrimidin-4-ylamino]-N-(3- | | |
| | {isopropyl-[2-(3,4,5-trihydroxy-6- | | |
| | hydroxymethyl-tetrahydro-pyran-2-yl)- | | |
| | ethyl]-amino}-propyl)-nicotinamide | | |
| 154 | 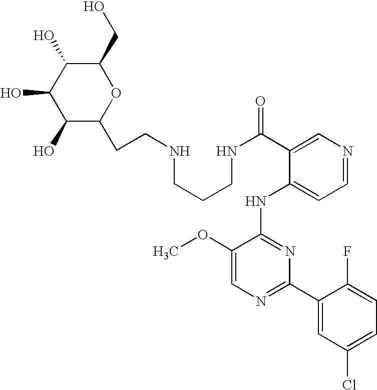 | 621.4, 1.05 | 0.01 |
| | AutoNom Name: | | |
| | 4-[2-(5-Chloro-2-fluoro-phenyl)-5- | | |
| | methoxy-pyrimidin-4-ylamino]-N-{3-[2- | | |
| | (3,4,5-trihydroxy-6-hydroxymethyl- | | |
| | tetrahydro-pyran-2-yl)-ethylamino]- | | |
| | propyl}-nicotinamide | | |
| 155 | 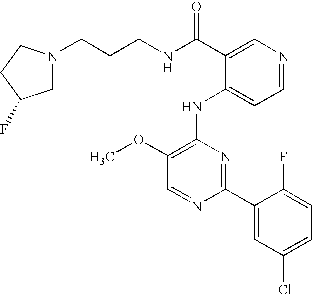 | 503.3, 1.12 | 0.008 |
| | AutoNom Name: | | |
| | 4-[2-(5-Chloro-2-fluoro-phenyl)-5- | | |
| | methoxy-pyrimidin-4-ylamino]-N-[3-(3- | | |
| | fluoro-pyrrolidin-1-yl)-propyl]- | | |
| | nicotinamide | | |
| 156 | 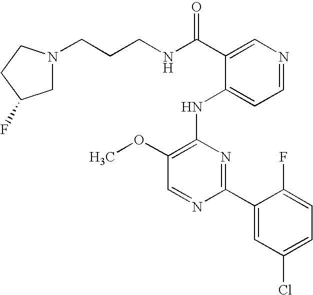 | 805.8, 1.45 | 0.198 |
| | AutoNom Name: | | |
| | 4-[2-(5-Chloro-2-fluoro-phenyl)-5- | | |
| | methoxy-pyrimidin-4-ylamino]-N-[3-(3- | | |
| | fluoro-pyrimidin-1-yl)-propyl]- | | |
| | nicotinamide | | |
| 157 | 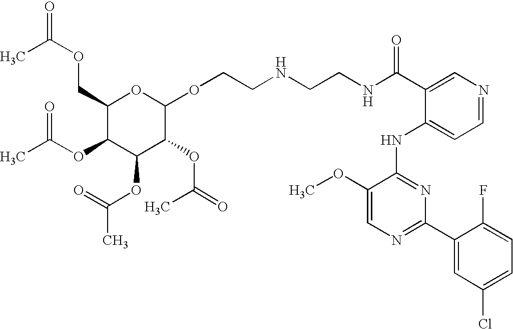 | 791.4, 1.27 | 0.025 |
| | AutoNom Name: | | |
| | Acetic acid 3,4,5-triacetoxy-6-{2-[2- | | |
| | [{4-[2-(5-chloro-2-fluoro-phenyl)- | | |
| | 5-methoxy-pyrimidin-4-ylamino]- | | |
| | pyridine-3-carbonyl}amino)-ethylamino]- | | |
| | ethoxy}-tetrahydro-pyran-2-ylmethyl- | | |
| | ester | | |
| 158 | 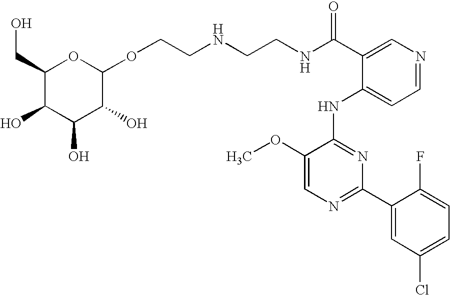 | 623.3, 1.01 | 0005 |
| | AutoNom Name: | | |
| | 4-[2-(5-Chloro-2-fluoro-phenyl)-5- | | |
| | methoxy-pyrimidin-4-ylamino]-N-{2-[2- | | |
| | (3,4,5-trihydroxy-4-hydroxymethyl- | | |
| | tetrahydro-pyran-2-yloxy)-ethylamino]- | | |
| | ethyl}-nicotinamide | | |
| 159 | 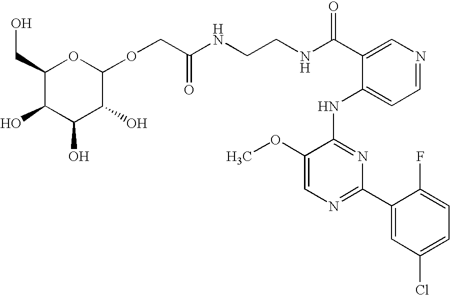 | 637.3, 1.07 | 0.058 |
| | AutoNom Name: | | |
| | 4-[2-(5-Chloro-2-fluoro-phenyl)-5- | | |
| | methoxy-pyrimidin-4-ylamino]-N-{2-[2- | | |
| | (3,4,5-trihydroxy-6-hydroxymethyl- | | |
| | tetrahydro-pyran-2-yloxy)-acetylamino]- | | |
| | ethyl}-nicotinamide | | |
| 160 | 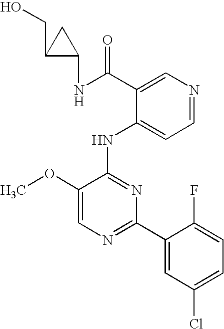 | 444.3, 1.16 | 0.009 |
| | AutoNom Name: | | |
| | 4-[2-(5-Chloro-2-fluoro-phenyl)-5- | | |
| | methoxy-pyrimidin-4-ylamino]-N-(2- | | |
| | hydroxymethyl-cyclopropyl)-nicotinamide | | |
| 161 | 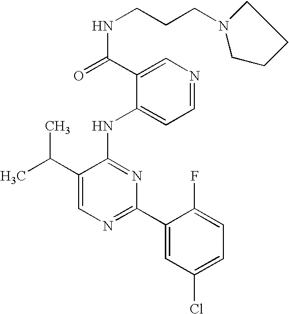 | 497.2, 1.30 | 0.004 |
| | AutoNom Name: | | |
| | 4-[2-(5-Chloro-2-fluoro-phenyl)-5- | | |
| | isopropyl-pyrimidin-4-ylamino]-N-(3- | | |
| | pyrrolidin-1-yl-propyl)-nicotinamide | | |
| 162 | 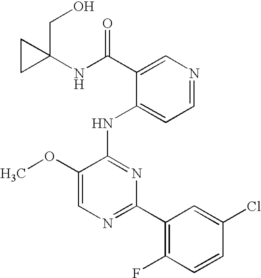 | 444.4, 1.21 | 0.042 |
| | AutoNom Name: | | |
| | 4-[2-(5-Chloro-2-fluoro-phenyl)-5- | | |
| | methoxy-pyrimidin-4-ylamino]-N-(1- | | |
| | hydroxymethyl-cyclopropyl)-nicotinamide | | |
| 163 | 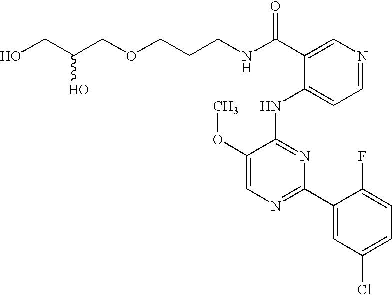 | 506.2, 1.19 | 0.033 |
| | AutoNom Name | | |
| | 4-[2-(5-Chloro-2-fluoro-phenyl)-5- | | |
| | methoxy-pyrimidin-4-ylamino]-N-[3-(2, | | |
| | 3-dihydroxy-propoxy)-propyl]-nicotinamide | | |
| 164 | 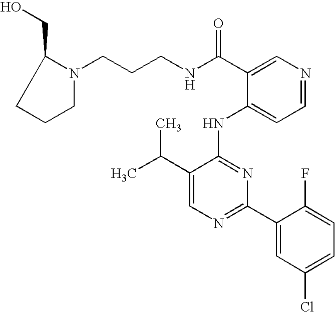 | 527.3, 1.27 | 0.002 |
| | AutoNom Name: | | |
| | 4-[2-(5-Chloro-2-fluoro-phenyl)-5- | | |
| | isopropyl-pyrimidin-4-ylamino]-N-[3- | | |
| | (2-hydroxymethyl-pyrrolidin-1-yl)- | | |
| | propyl]-nicotinamide | | |
| 165 | 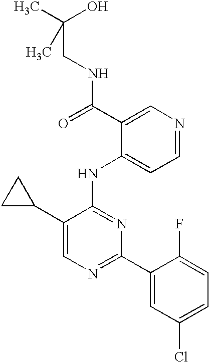 | 456.0, 1.46 | 0.027 |
| | AutoNom Name: | | |
| | 4-[2-(5-Chloro-2-fluoro-phenyl)-5- | | |
| | cyclopropyl-pyrimidin-4-ylamino]-N- | | |
| | 2-hydroxy-2-methyl-propyl)-nicotinamide | | |
| 166 | 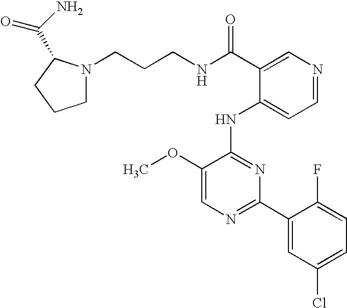 | 528.4, 1.10 | 0.005 |
| | AutoNom Name: | | |
| | N-[3-(2-Carbamoyl-pyrrolidin-1-yl)- | | |
| | propyl]-4-[2-(5-chloro-2-fluoro- | | |
| | phenyl)-5-methoxy-pyrimidin-4-ylamino]- | | |
| | nicotinamide | | |
| 167 | 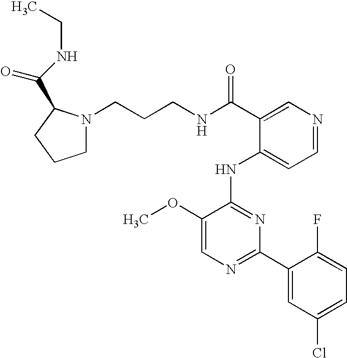 | 556.4, 1.16 | 0.028 |
| | AutoNom Name: | | |
| | 4-[2-(5-Chloro-2-fluoro-phenyl)-5- | | |
| | methoxy-pyrimidin-4-ylamino]-N-[3-(2- | | |
| | ethylcarbamoyl-pyrrolidin-1-yl)- | | |
| | propyl]-nicotinamide | | |
| 168 | 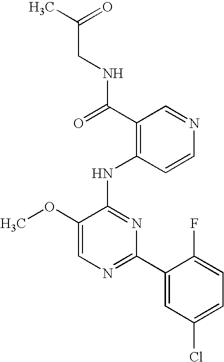 | 430.3, 1.25 | 0.024 |
| | AutoNom Name: | | |
| | 4-[2-(5-Chloro-2-fluoro-phenyl)-5- | | |
| | methoxy-pyrimidin-4-ylamino]-N-(2-oxo- | | |
| | propyl)-nicotinamide | | |
| 169 | 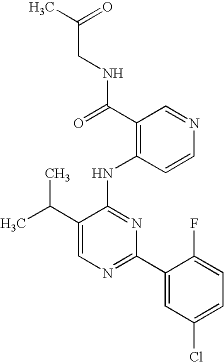 | 442.1, 1.43 | 0.003 |
| | AutoNom Name: | | |
| | 4-[2-(5-Chloro-2-fluoro-phenyl)-5- | | |
| | isopropyl-pyrimidin-4-ylamino]-N-(2- | | |
| | oxo-propyl)-nicatinamide | | |
| 170 | 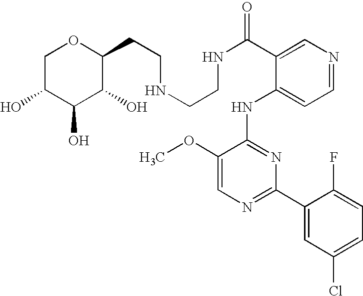 | 577.2, 1.04 | 0.006 |
| | AutoNom Name: | | |
| | 4-[2-(5-Chloro-2-fluoro-phenyl)-5- | | |
| | methoxy-pyrimidin-4-ylamino]-N-{2-[2- | | |
| | (3,4,5-trihydroxy-tetrahydro-pyran- | | |
| | 2-yl)-ethylamino]-ethyl}-nicotinamide | | |
| 171 | 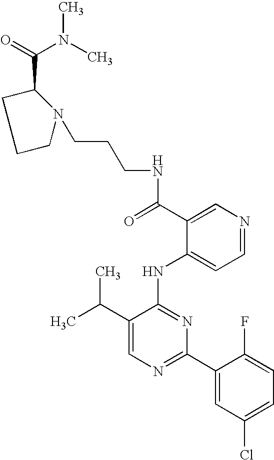 | 568.6, 1.25 | 0.005 |
| | AutoNom Name: | | |
| | 4-[2-(5-Chloro-2-fluoro-phenyl)-5- | | |
| | isopropyl-pyrimidin-4-ylamino]-N-[3- | | |
| | (2-dimethylcarbamoyl-pyrrolidin-1-yl)- | | |
| | propyl]-nicotinamide | | |
| 172 | 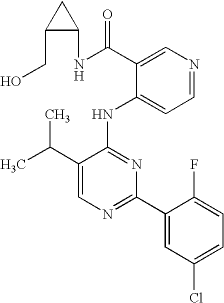 | 456.0, 1.40 | 0.005 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxymethyl-cyclopropyl)-nicotinamide | | |
| 173 | 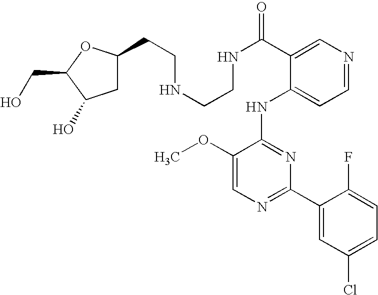 | 561.2, 1.08 | 0.006 |
| | AutoNom Name: | | |
| | 4-[2-(5-Chloro-2-fluoro-phenyl)-5- | | |
| | methoxy-pyrimidin-4-ylamino]-N-{2-[2- | | |
| | (4-hydroxy-5-hydroxymethyl-tetrahydro- | | |
| | furan-2-yl)-ethylamino]-ethyl}- | | |
| | nicotinamide | | |
| 174 | 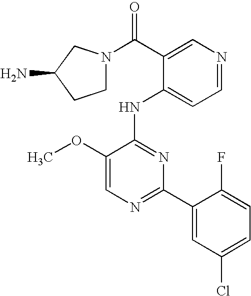 | 443.4, 1.05 | 1.163 |
| | AutoNom Name: | | |
| | (3-Amino-pyrrolidin-1-yl)-{4-[2-(5- | | |
| | chloro-2-fluoro-phenyl)-5-methoxy- | | |
| | pyrimidin-4-ylamino]-pyridin-3-yl}- | | |
| | methanone | | |
| 175 | 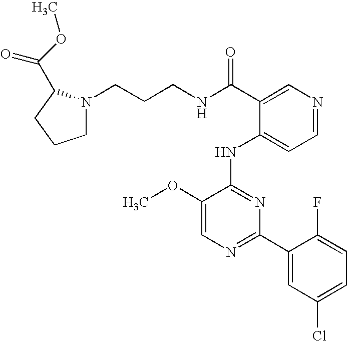 | 543.5, 1.17 | 0.031 |
| | AutoNom Name: | | |
| | 1-[3-({4-[2-(5-Chloro-2-fluoro-phenyl)- | | |
| | 5-methoxy-pyrimidin-4-ylamino]- | | |
| | pyridine-3-carbonyl}-amino)-propyl]- | | |
| | pyrrolidine-2-carboxylic acid methyl | | |
| | ester | | |
| 176 | 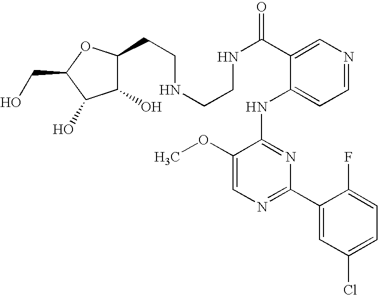 | 577.2, 1.03 | 0.005 |
| | AutoNom Name: | | |
| | 4-[2-(5-Chloro-2-fluoro-phenyl)-5- | | |
| | methoxy-pyrimidin-4-ylamino]-N-{2-[2- | | |
| | (3,4-dihydroxy-5-hydroxymethyl- | | |
| | tetrahydro-furan-2-yl)-ethylamino]-ethyl}- | | |
| | nicotinamide | | |
| 177 | 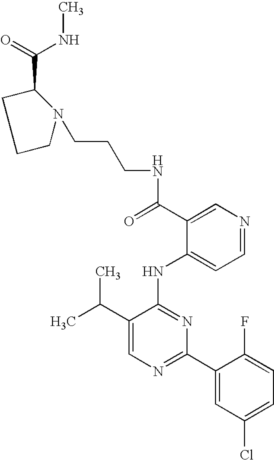 | 554.1, 1.26 | 0.011 |
| | AutoNom Name: | | |
| | 4-[2-(5-Chloro-2-fluoro-phenyl)-5- | | |
| | isopropyl-pyrimidin-4-ylamino]-N-[3- | | |
| | (2-methylcarbamoyl-pyrrolidin-1-yl)- | | |
| | propyl]-nicotinamide | | |
| 178 | 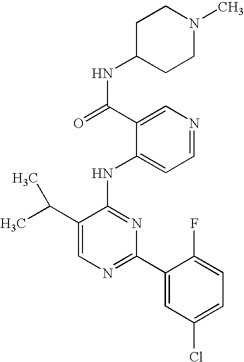 | 483.4, 1.23 | 0.003 |
| | AutoNom Name: | | |
| | 4-[2-(5-Chloro-2-fluoro-phenyl)-5- | | |
| | isopropyl-pyrimidin-4-ylamino]-N-(1- | | |
| | methyl-piperidin-4-yl)-nicotinamide | | |
| 179 | 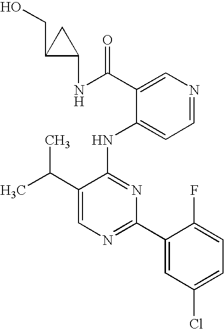 | 456.0, 1.41 | 0.005 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxymethyl-cyclopropyl)-nicotinamide | | |
| 180 | 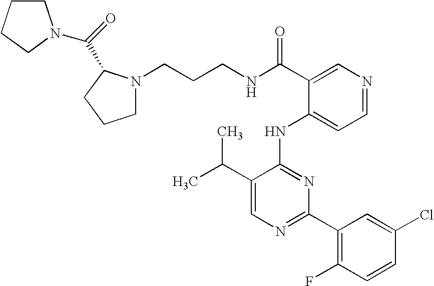 | 594.5, 1.33 | 0.005 |
| | AutoNom Name: | | |
| | 4-[2-(5-Chloro-2-fluoro-phenyl)-5- | | |
| | isoopropyl-pyrimidin-4-ylamino]-N-{3- | | |
| | [2-pyrrolidin-1-carbonyl)-pyrrolidin- | | |
| | 1-yl]-propyl}-nicotinamide | | |
| 181 | 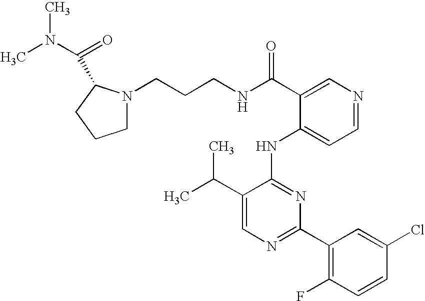 | 568.5, 1.28 | 0.006 |
| | AutoNom Name: | | |
| | 4-[2-(5-Chloro-2-fluoro-phenyl)-5- | | |
| | isopropyl-pyrimidin-4-ylamino]-N-[3- | | |
| | (2-dimethylcarbamoyl-pyrrolidin-1-yl)- | | |
| | propyl]-nicotinamide | | |
| 182 | 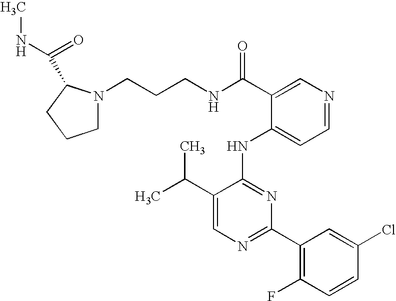 | 554.5, 1.26 | 0.011 |
| | AutoNom Name: | | |
| | 4-[2-(5-Chloro-2-fluoro-phenyl)-5- | | |
| | isopropyl-pyrimidin-4-ylamino]-N-[3- | | |
| | (2-methylcarbamoyl-pyrrolidin-1-yl)- | | |
| | propyl]-nicotinamide | | |
| 183 | 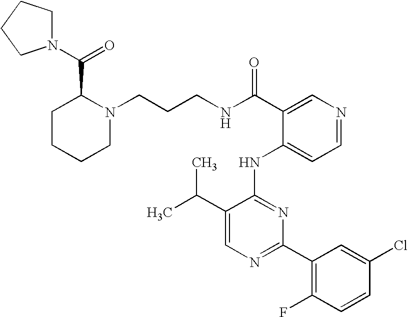 | 608.4, 1.34 | 0.021 |
| | AutoNom Name: | | |
| | 4-[2-(5-Chloro-2-fluoro-phenyl)-5- | | |
| | isopropyl-pyrimidin-4-ylamino]-N-{3- | | |
| | [2-(pyrrolidine-1-carbonyl)-piperidin- | | |
| | 1-yl]-propyl}-nicotinamide | | |
| 184 | 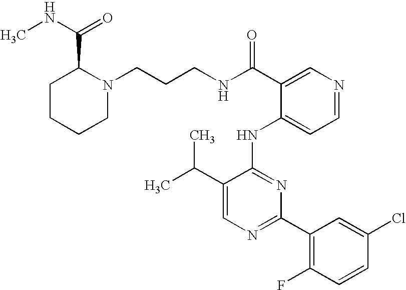 | 568.4, 1.28 | 0.012 |
| | AutoNom Name: | | |
| | 4-[2-(5-Chloro-2-fluoro-phenyl)-5- | | |
| | isopropyl-pyrimidin-4-ylamino]-N-[3- | | |
| | (2-methylcarbamoyl-piperidin-1-yl)- | | |
| | propyl}-nicotinamide | | |
| 185 | 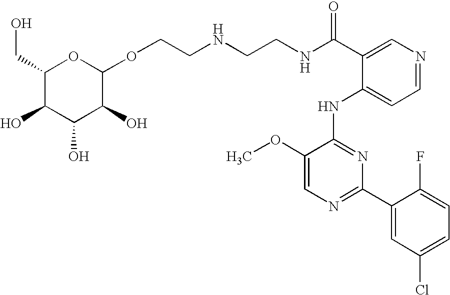 | 623.3, 1.04 | 0.009 |
| | AutoNom Name: | | |
| | 4-[2-(5-Chloro-2-fluoro-phenyl)-5- | | |
| | methoxy-pyrimidin-4-ylamino]-{2-]2- | | |
| | (3,4,5-trihydroxy-6-hydroxymethyl- | | |
| | tetrahydro-pyran-2-yloxy)-ethylamino]- | | |
| | ethyl}-nicotinamide | | |
| 186 | 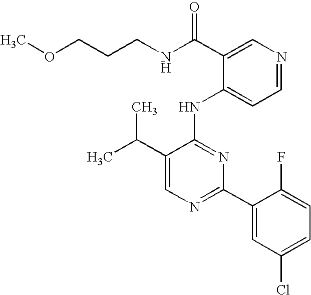 | 458.2, 1.53 | 0.011 |
| | AutoNom Name: | | |
| | 4-[2-(5-Chloro-2-fluoro-phenyl)-5- | | |
| | isopropyl-pyrimidin-4-ylamino]-N-(3- | | |
| | methoxy-propyl)-nicotinamide | | |
| 187 | 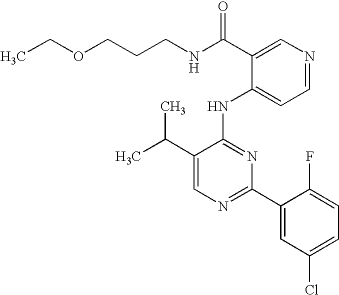 | 472.2, 1.99 | 0.012 |
| | AutoNom Name: | | |
| | 4-[2-(5-Chloro-2-fluoro-phenyl)-5- | | |
| | isopropyl-pyrimidin-4-ylamino]-N-(3- | | |
| | ethoxy-propyl)-nicotinamide | | |
| 188 | 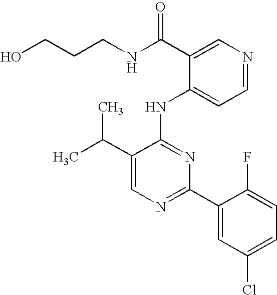 | 444.1, 1.37 | 0.005 |
| | AutoNom Name: | | |
| | 4-[2-(5-Chloro-2-fluoro-phenyl)-5- | | |
| | isopropyl-pyrimidin-4-ylamino]-N-(3- | | |
| | hydroxy-propyl)-nicotinamide | | |
| 189 | 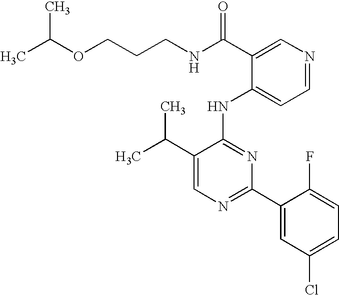 | 486.3, 1.65 | 0.074 |
| | AutoNom Name: | | |
| | 4-[2-(5-Chloro-2-fluoro-phenyl)-5- | | |
| | isopropyl-pyrimidin-4-ylamino]-N-(3- | | |
| | isopropoxy-propyl)-nicotinamide | | |
| 190 | 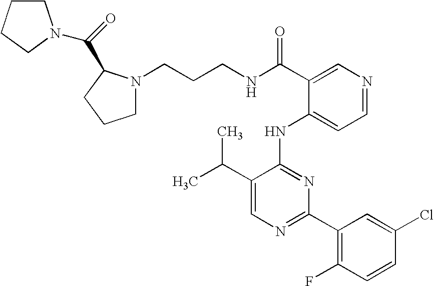 | 593.9, 1.35 | 0.009 |
| | AutoNom Name: | | |
| | 4-[2-(5-Chloro-2-fluoro-phenyl)-5- | | |
| | isopropyl-pyrimidin-4-ylamino]-N-{3- | | |
| | [2-(pyrrolidine-1-carbonyl)-pyrrolidine- | | |
| | 1-yl]-propyl}-nicotinamide | | |
| 191 | 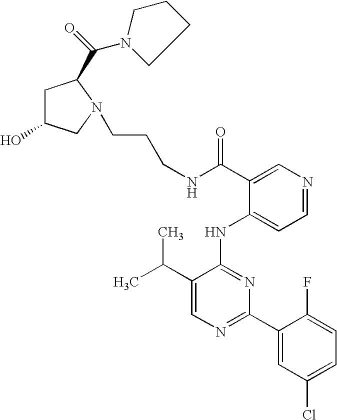 | 610.5, 1.28 | 0.013 |
| | AutoNom Name: | | |
| | 4-[2-(5-Chloro-2-fluoro-phenyl)-5- | | |
| | isopropyl-pyrimidin-4-ylamino]-N-{3- | | |
| | [4-hydroxy-2-(pyrrolidine-1-carbonyl)- | | |
| | pyrrolidin-1-yl]-propyl}-nicotinamide | | |
| 192 | 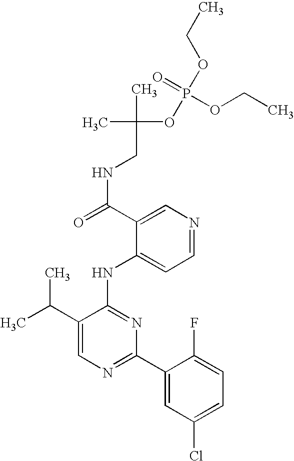 | 594.3, 1.67 | 0.122 |
| | AutoNom Name: | | |
| | Phosphonic acid 2-({4-[2-(5-chloro- | | |
| | 2-fluoro-phenyl)-5-isopropyl-pyrimidin- | | |
| | 4-ylamino]-pyridine-4-carbonyl}- | | |
| | amino)-1,1-dimethyl-ethyl ester | | |
| | diethyl ester | | |
| 193 | 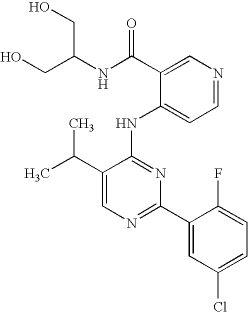 | 460.2, 1.25 | 0.027 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxy-1-hydroxymethyl-ethyl)-nicotinamide | | |
| 194 | 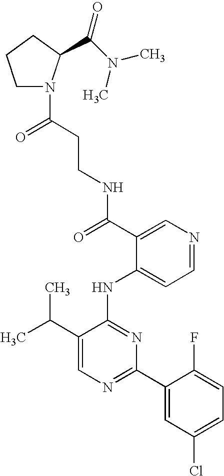 | 582.5, 1.40 | 0.041 |
| | AutoNom Name: | | |
| | 4-[2-(5-Chloro-2-fluoro-phenyl)-5- | | |
| | isopropyl-pyrimidin-4-ylamino]-N-[3- | | |
| | (2-dimethyl-carbamoyl-pyrrolidin-1-yl)- | | |
| | 3-oxo-propyl]-nicotinamide | | |
| 195 | 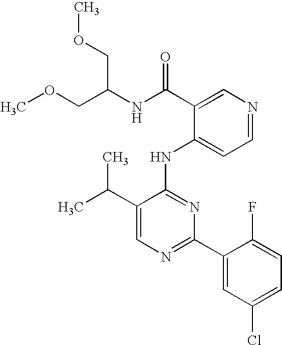 | 488.4, 1.53 | 0.424 |
| | AutoNom Name: | | |
| | 4-[2-(5-Chloro-2-fluoro-phenyl)-5- | | |
| | isopropyl-pyrimidin-4-ylamino]-N-(2- | | |
| | methoxy-1-methoxymethyl-ethyl)- | | |
| | nicotinamide | | |
| 196 | 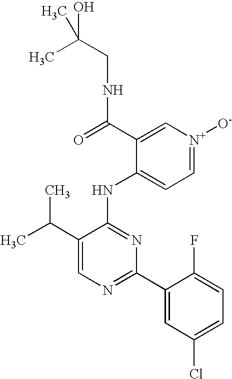 | 474.0, 1.40 | 0.245 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxy-2-methyl-propyl)-1-oxy- | | |
| | nicotinamide | | |
| 197 | 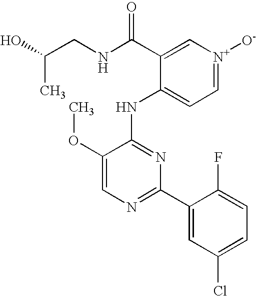 | 447.9, 1.20 | 0.675 |
| | AutoNom Name: | | |
| | 4-[2-(5-Chloro-2-fluoro-phenyl)-5- | | |
| | methoxy-pyrimidin-4-ylamino]-N-(2- | | |
| | hydroxy-propyl)-1-oxy-nicotinamide | | |
| 198 | 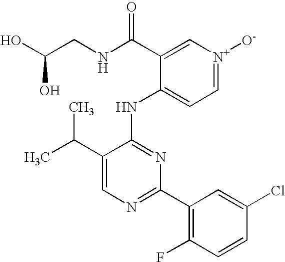 | 460.0, 1.33 | 0.148 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxy-propyl)-1-oxy-nicotinamide | | |
| 199 | 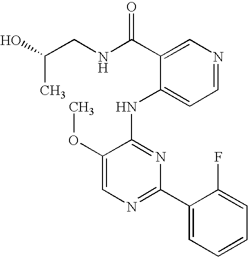 | | |
| | AutoNom Name: | | |
| | 4-[2-(2-Fluoro-phenyl)-5-methoxy- | | |
| | pyrimidin-4-ylamino]-N-(2-hydroxy- | | |
| | propyl)-nicotinamide | | |
| 200 | 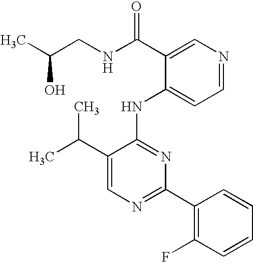 | | |
| | AutoNom Name: | | |
| | 4-[2-(2-Fluoro-phenyl)-5-isopropyl- | | |
| | pyrimidin-4-ylamino]-N-(2-hydroxy- | | |
| | propyl)-nicotinamide | | |
| 201 | 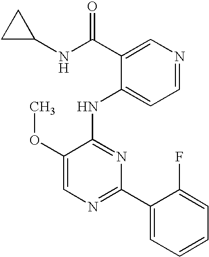 | | |
| | AutoNom Name: | | |
| | N-Cyclopropyl-4-[2-(2-fluoro-phenyl)- | | |
| | 5-methoxy-pyrimidin-4-ylamino]- | | |
| | nicotinamide | | |
| 202 | 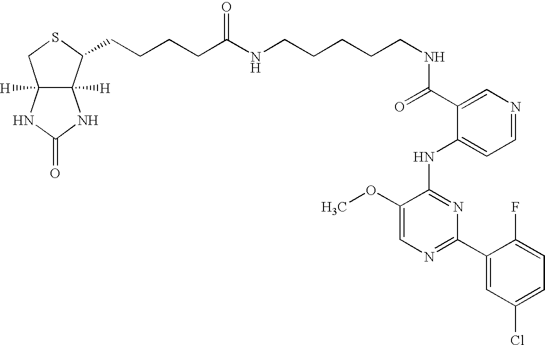 | 685.1, 1.32 | 0054 |
| | AutoNom Name: | | |
| | 4-[2-(5-Chloro-2-fluoro-phenyl)-5- | | |
| | methoxy-pyrimidin-4-ylamino]-N-{5-[5- | | |
| | (2-oxo-hexahydro-thieno[3,4-d]imidazol- | | |
| | 6-yl)-pentanoylamino]-pentyl]- | | |
| | nicotinamide | | |
| 203 | 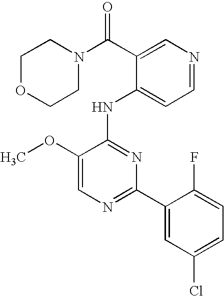 | 444.0, 1.21 | 0.176 |
| | AutoNom Name: | | |
| | {4-[2-(5-Chloro-2-fluoro-phenyl)-5- | | |
| | methoxy-pyrimidin-4-ylamino]-pyridin- | | |
| | 3-yl}-morpholin-4-yl-methanone | | |
| 204 | 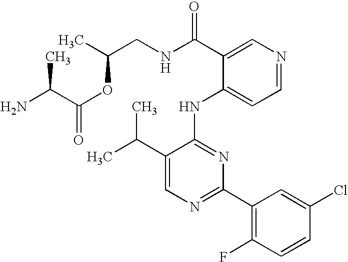 | 515.3, 1.31 | 0.025 |
| | AutoNom Name: | | |
| | 2-Amino-propionic acid 2-({4-[2-(5- | | |
| | chloro-2-fluoro-phenyl)-5-isopropyl- | | |
| | pyrimidin-4-ylamino]-pyridine-3- | | |
| | carbonyl}-amino)-1-methyl-ethyl ester | | |
| 205 | 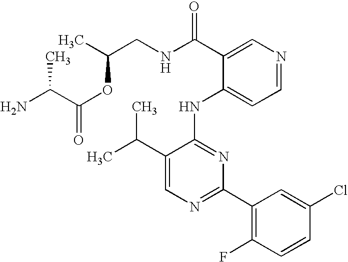 | 515.0, 1.31 | 0.01 |
| | AutoNom Name: | | |
| | 2-Amino-propionic acid 2-({4-[2-(5- | | |
| | chloro-2-fluoro-phenyl)-5-isopropyl- | | |
| | pyrimidin-4-ylamino]-pyridine-3- | | |
| | carbonyl}-amino)-1-methyl-ethyl ester | | |
| 206 | 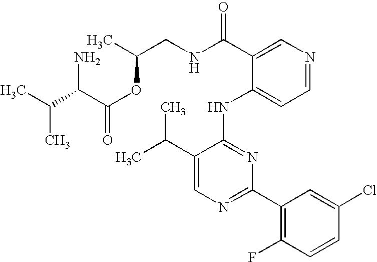 | 543.4, 1.31 | 0.072 |
| | AutoNom Name: | | |
| | 2-Amino-3-methyl-butyric acid 2-({4- | | |
| | [2-(5-chloro-2-fluoro-phenyl)-5- | | |
| | isopropyl-pyrimidin-4-ylamino]-pyridine- | | |
| | 3-carbonyl}-amino)-1-methyl-ethyl | | |
| | ester | | |
| 207 | 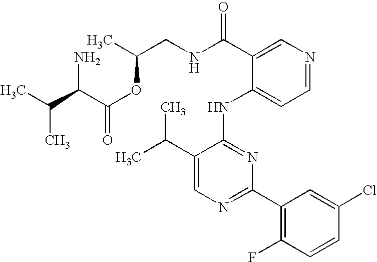 | 543.4, 1.35 | 0.017 |
| | AutoNom Name: | | |
| | 2-Amino-3-methyl-butyric acid 2-({4- | | |
| | [2-(5-chloro-2-fluoro-phenyl)-5- | | |
| | isopropyl-pyrimidin-4-ylamino]-pyridine- | | |
| | 3-carbonyl}-amino)-1-methyl-ethyl- | | |
| | ester | | |
| 208 | 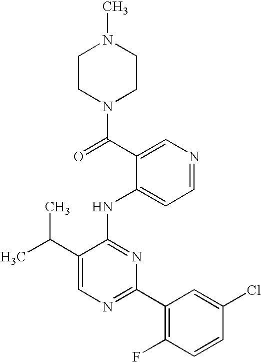 | 469.1, 1.19 | 1.617 |
| | AutoNom Name: | | |
| | {4-[2-(5-Chloro-2-fluoro-phenyl)-5- | | |
| | isopropyl-pyrimidin-4-ylamino]- | | |
| | pyridin-3-yl}-(4-methyl-piperazin-1-yl)- | | |
| | methanone | | |
| 209 | 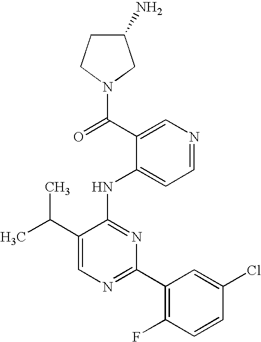 | 455.4, 1.13 | 0.068 |
| | AutoNom Name: | | |
| | (3-Amino-pyrrolidin-1-yl)-{4-[2-(5- | | |
| | chloro-2-fluoro-phenyl)-5-isopropyl- | | |
| | pyrimidin-4-ylamino]-pyridin-3-yl}- | | |
| | methanone | | |
| 210 | 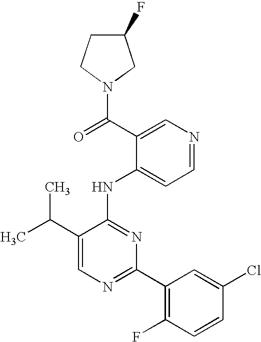 | 457.9, 1.52 | 0.121 |
| | AutoNom Name: | | |
| | {4-[2-(5-Chloro-2-fluoro-phenyl)-5- | | |
| | isopropyl-pyrimidin-4-ylamino]-pyridin- | | |
| | 3-yl}-(3-fluoro-pyrrolidin-1-yl)- | | |
| | methanone | | |
| 211 | 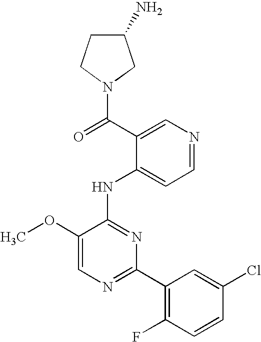 | 443.3, 1.05 | 0.258 |
| | AutoNom Name: | | |
| | (3-Amino-pyrrolidin-1-yl)-{4-[2-(5- | | |
| | chloro-2-fluoro-phenyl)-5-methoxy- | | |
| | pyrimidin-4-ylamino]-pyridin-3-yl)- | | |
| | methanone | | |
| 212 | 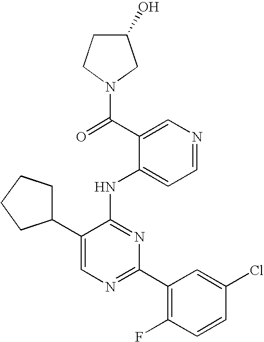 | 481.8, 1.49 | 0.673 |
| | AutoNom Name: | | |
| | {4-[2-(5-chloro-2-fluoro-phenyl)-5- | | |
| | cyclopentyl-pyrimidin-4-ylamino]- | | |
| | pyridin-3-yl}(3-hydroxy-pyrrolidin-1- | | |
| | yl)-methanone | | |
| 213 | 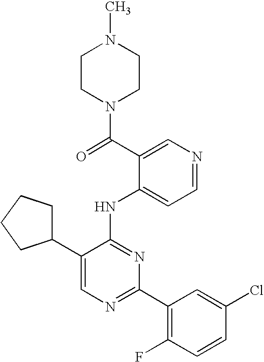 | 494.9, 1.32 | 12.892 |
| | AutoNom Name: | | |
| | {4-[2-(5-Chloro-2-fluoro-phenyl)-5- | | |
| | cyclopentyl-pyrimidin-4-ylamino]- | | |
| | pyridin-3-yl}-(4-methyl-piperazin-1-yl)- | | |
| | methanone | | |
| 214 | 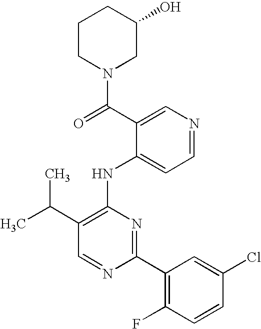 | 470.0, 1.37 | 0.283 |
| | AutoNom Name: | | |
| | {4-[2-(5-Chloro-2-fluoro-phenyl)-5- | | |
| | isopropyl-pyrimidin-4-ylamino]- | | |
| | pyridin-3-yl}-(3-hydroxy-piperidin-1-yl)- | | |
| | methanone | | |
| 215 | 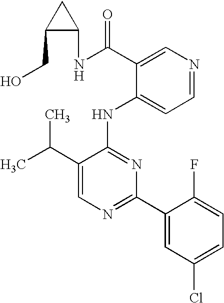 | 456.0, 1.39 | 0.005 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxymethyl-cyclopropyl)-nicotinamide | | |
| 216 | 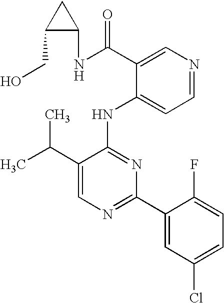 | 456.0, 1.40 | 0.01 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxymethyl-cyclopropyl)-nicotinamide | | |
| 217 | 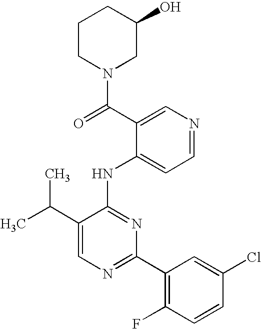 | 470.4, 1.20 | 1.208 |
| | AutoNom Name: | | |
| | {4-[2-(5-Chloro-2-fluoro-phenyl)-5- | | |
| | isopropyl-pyrimidin-4-ylamino]- | | |
| | pyridin-3-yl}-(3-hydroxy-piperidin-1-yl)- | | |
| | methanone | | |
| 218 | 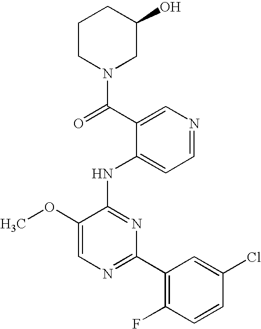 | 458.0, 1.21 | 1.777 |
| | AutoNom Name: | | |
| | {4-[2-(5-Chloro-2-fluoro-phenyl)-5- | | |
| | methoxy-pyrimidin-4-ylamino]-pyridin- | | |
| | 3-yl}-(3-hydroxy-piperidin-1-yl)- | | |
| | methanone | | |
| 219 | 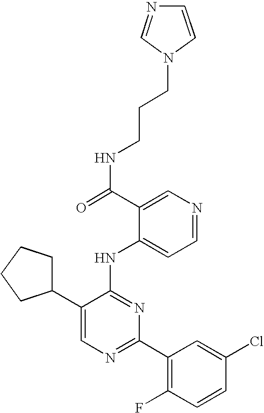 | 520.3, 1.39 | 0.044 |
| | AutoNom Name: | | |
| | 4-[2-(5-Chloro-2-fluoro-phenyl)-5- | | |
| | cyclopentyl-pyrimidin-4-ylamino]-N- | | |
| | 3-imidazol-1-yl-propyl)-nicotinamide | | |
| 220 | 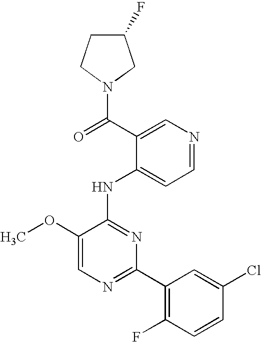 | 446.0, 1.31 | 0.249 |
| | AutoNom Name: | | |
| | {4-[2-(5-Chloro-2-fluoro-phenyl)-5- | | |
| | methoxy-pyrimidin-4-ylamino]-pyridin- | | |
| | 3-yl}-(3-fluoro-pyrrolidin-1-yl)- | | |
| | methanone | | |
| 221 | 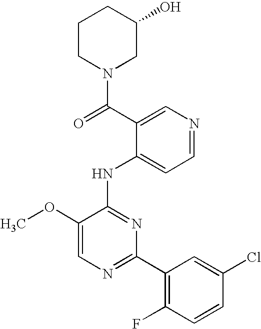 | 458.1, 1.33 | 1.456 |
| | AutoNom Name: | | |
| | {4-[2-(5-Chloro-2-fluoro-phenyl)-5- | | |
| | methoxy-pyrimidin-4-ylamino]-pyridin- | | |
| | 3-yl}-(3-hydroxy-piperidin-1-yl)- | | |
| | methanone | | |
| 222 | 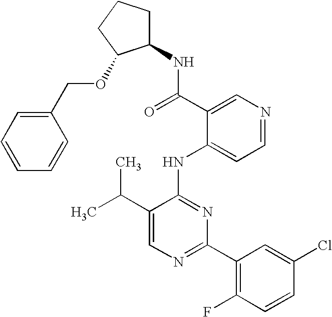 | 560.1, 2.00 | 7.696 |
| | AutoNom Name: | | |
| | N-(2-Benzyloxy-cyclopentyl)-4-[2-(5- | | |
| | chloro-2-fluoro-phenyl)-5-isopropyl- | | |
| | pyrimidin-4-ylamino]-nicotinamide | | |
| 223 | 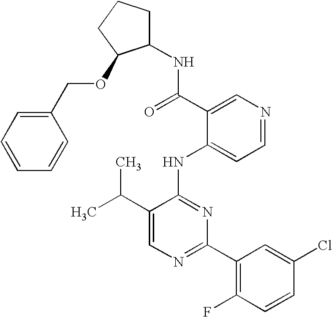 | 560.1, 1.59 | 7.141 |
| | AutoNom Name: | | |
| | N-(2-Benzyloxy-cyclopentyl)-4-[2-(5- | | |
| | chloro-2-fluoro-phenyl)-5-isopropyl- | | |
| | pyrimidin-4-ylamino]-nicotinamide | | |
| 224 | 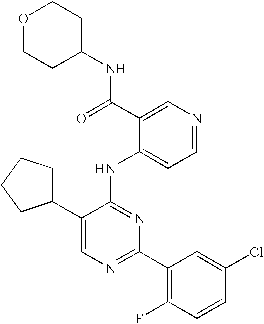 | | |
| | AutoNom Name: | | |
| | 4-[2-(5-Chloro-2-fluoro-phenyl)-5- | | |
| | cyclopentyl-pyrimidin-4-ylamino]-N- | | |
| | tetrahydro-pyran-4-yl)-nicotinamide | | |
| 225 | 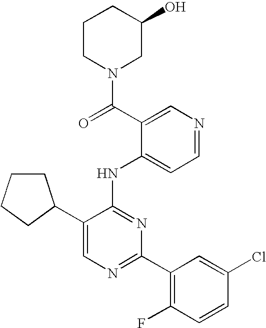 | 496.1, 1.53 | 1.394 1.804 |
| | AutoNom Name: | | |
| | {4-[2-(5-Chloro-2-fluoro-phenyl)-5- | | |
| | cyclopentyl-pyrimidin-4-ylamino]- | | |
| | pyridin-3-yl}-(3-hydroxy-piperidin-1- | | |
| | yl)-methanone | | |
| 226 | 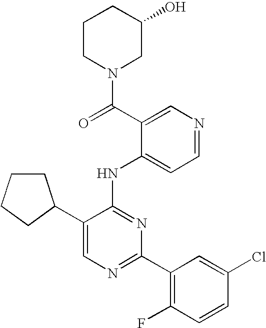 | 496.2, 1.53 | 2.47 |
| | AutoNom Name: | | |
| | {4-[2-(5-Chloro-2-fluoro-phenyl)-5- | | |
| | cyclopentyl-pyrimidin-4-ylamino]- | | |
| | pyridin-3-yl}-(3-hydroxy-piperidin-1- | | |
| | yl)-methanone | | |
| 227 | 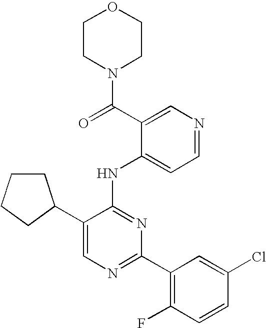 | 481.7, 1.59 | 0.33 |
| | AutoNom Name: | | |
| | {4-[2-(5-Chloro-2-fluoro-phenyl)-5- | | |
| | cyclopentyl-pyrimidin-4-ylamino]- | | |
| | pyridin-3-yl}-morpholin-4-yl-methanone | | |
| 228 | 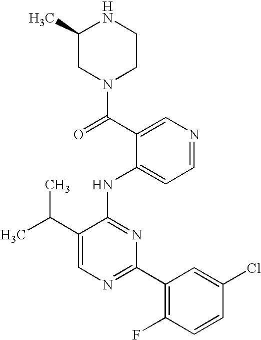 | 469.1, 1.24 | 2.781 |
| | AutoNom Name: | | |
| | {4-[2-(5-Chloro-2-fluoro-phenyl)-5- | | |
| | isopropyl-pyrimidin-4-ylamino]-pyridin- | | |
| | 3-yl}-(3-methyl-piperazin-1-yl)- | | |
| | methanone | | |
| 229 | 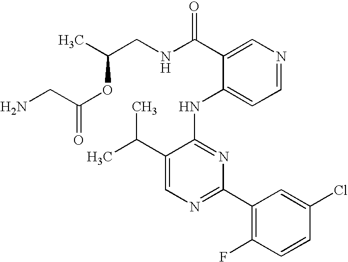 | 501.2, 1.31 | 0.019 |
| | AutoNom Name: | | |
| | Amino-acetic acid 2-{[4-[2-(5-chloro- | | |
| | 2-fluoro-phenyl)-5-isopropyl-pyrimidin- | | |
| | 4-ylamino]-pyridine-3-carbonyl}- | | |
| | amino]-1-methyl-ethyl ester | | |
| 230 | 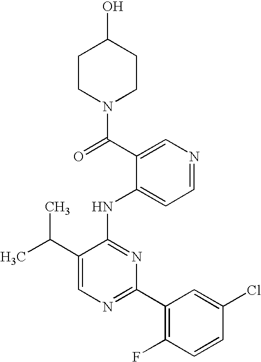 | 470.6, 1.34 | 1.139 |
| | AutoNom Name: | | |
| | {4-[2-(5-Chloro-2-fluoro-phenyl)-5- | | |
| | isopropyl-pyrimidin-4-ylamino)-pyridin- | | |
| | 3-yl}-(4-hydroxy-piperidin-1-yl)- | | |
| | methanone | | |
| 231 | 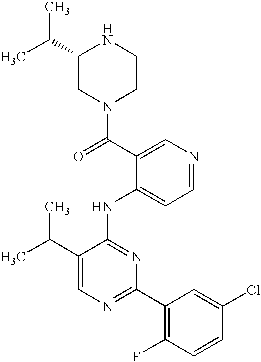 | 497.2, 1.23 | 1.088 |
| | AutoNom Name: | | |
| | {4-[2-(5-Chloro-2-fluoro-phenyl)-5- | | |
| | isopropyl-pyrimidin-4-ylamino]-pyridin- | | |
| | 3-yl}-(3-isopropyl-piperazin-1- | | |
| | yl)-methanone | | |
| 232 | 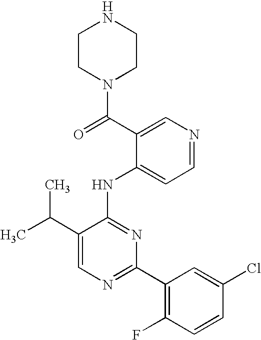 | 455.3, 1.15 | 0.99 |
| | AutoNom Name: | | |
| | {4-[2-(5-Chloro-2-fluoro-phenyl)-5- | | |
| | isopropyl-pyrimidin-4-ylamino]-pyridin- | | |
| | 3-yl}-piperazin-1-yl-methanone | | |
| 233 | 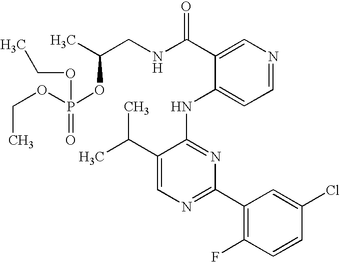 | 580.3, 1.55 | 0.28 |
| | AutoNom Name: | | |
| | Phosphoric acid 2-{(4-[2-(5-chloro- | | |
| | 2-fluoro-phenyl)-5-isopropyl-pyrimidin- | | |
| | 4-ylamino]-pyridine-3-carbonyl}- | | |
| | amino)-1-methyl-ethyl ester diethyl | | |
| | ester | | |
| 234 | 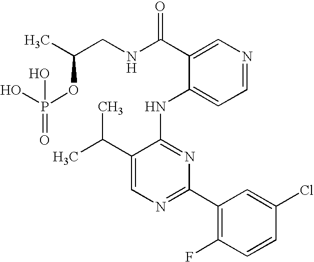 | 524.2, 1.31 | 0.178 |
| | AutoNom Name: | | |
| | Phosphoric acid mono-[2-{[4-[2-(5- | | |
| | chloro-2-fluoro-phenyl}-5-isopropyl- | | |
| | pyrimidin-4-ylamino]-pyridine-3- | | |
| | carbony}-amino)-1-methyl-ethyl] ester | | |
| 235 | 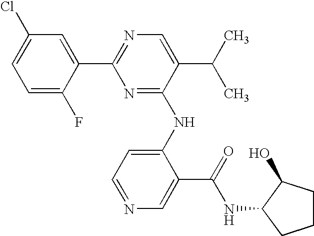 | 470.3, 1.69 | 0.04 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxy-cyclopentyl)-nicotinamide | | |
| 236 | 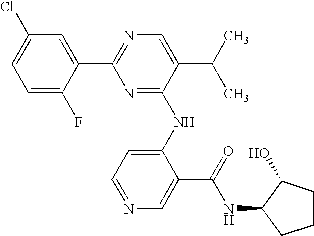 | 470.3, 1.67 | 0.123 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxy-cyclopentyl)-nicotinamide | | |
| 237 | 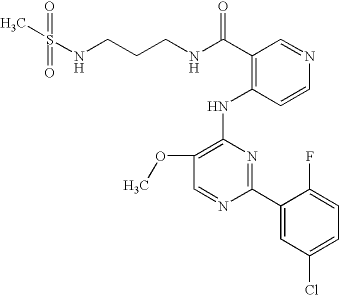 | 509.0, 1.56 | 0.063 |
| | AutoNom Name: | | |
| | 4-[2-(5-Chloro-2-fluoro-phenyl)-5- | | |
| | methoxy-pyrimidin-4-ylamino]-N-(3- | | |
| | methanesulfonylamino-propyl)-nicotinamide | | |
| 238 | 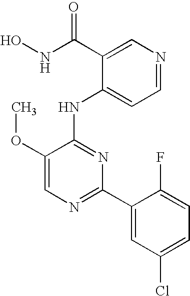 | 390.1, 1.17 | 1.078 |
| | AutoNom Name: | | |
| | 4-[2-(5-Chloro-2-fluoro-phenyl)-5- | | |
| | methoxy-pyrimidine-4-ylamino]-N-hydroxy- | | |
| | nicotinamide | | |
| 239 | 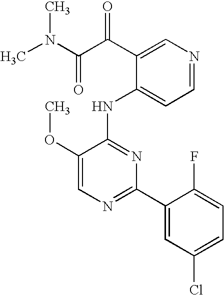 | 430.3, 1.34 | 7.74 |
| | AutoNom Name: | | |
| | 2-{4-[2-(5-Chloro-2-fluoro-phenyl)- | | |
| | 5-methoxy-pyrimidin-4-ylamino]- | | |
| | pyridin-3-yl]-N,N-dimethyl-2-oxo- | | |
| | acetamide | | |
| 240 | 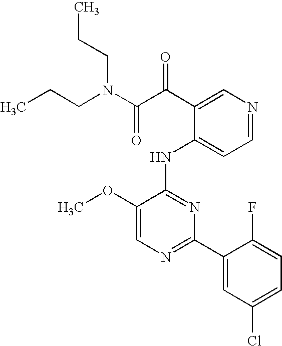 | 486.1, 1.65 | 3.085 |
| | AutoNom Name: | | |
| | 2-{4-[2-(5-Chloro-2-fluoro-phenyl)- | | |
| | 5-methoxy-pyrimidin-4-ylamino]- | | |
| | pyridin-3-yl}-2-oxo-N,N-dipropyl-acetamide | | |
| 241 | 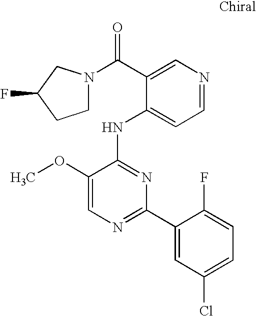 | 446.0, 1.29 | 0.127 |
| | AutoNom Name: | | |
| | {4-[2-(5-Chloro-2-fluoro-phenyl)-5- | | |
| | methoxy-pyrimidin-4-ylamino]-pyridin- | | |
| | 3-yl}-(3-fluoro-pyrrolidin-1-yl)- | | |
| | methanone | | |
| 242 | 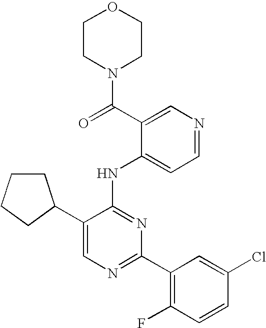 | 481.7, 1.59 | 0.33 |
| | AutoNom Name: | | |
| | {4-[2-(5-Chloro-2-fluoro-phenyl)-5- | | |
| | cyclopentyl-pyrimidin-4-ylamino]- | | |
| | pyridin-3-yl}-morpholin-4-yl-methanone | | |
| 243 | 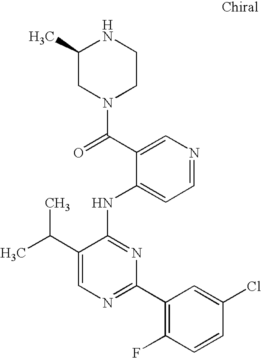 | 469.3, 1.24 | 2.781 |
| | AutoNom Name: | | |
| | {4-[2-(5-Chloro-2-fluoro-phenyl)-5- | | |
| | isopropyl-pyrimidin-4-ylamino]-pyridin- | | |
| | 3-yl}-(3-methyl-piperazin-1-yl)- | | |
| | methanone | | |
| 244 | 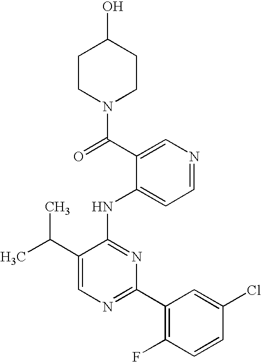 | 470.2, 1.34 | 1.139 |
| | AutoNom Name: | | |
| | {4-[2-(5-Chloro-2-fluoro-phenyl)-5- | | |
| | isopropyl-pyrimidin-4-ylamino]- | | |
| | pyridin-3-yl}-(4-hydroxy-piperidin-1-yl)- | | |
| | methanone | | |
| 245 | 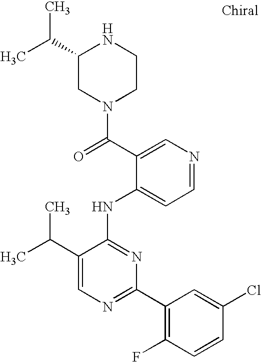 | 497.2, 1.23 | 1.088 |
| | AutoNom Name: | | |
| | {4-[2-(5-Chloro-2-fluoro-phenyl)-5- | | |
| | isopropyl-pyrimidin-4-ylamino]-pyridin- | | |
| | 3-yl}-(3-isopropyl-piperazin-1- | | |
| | yl)-methanone | | |
| 246 | 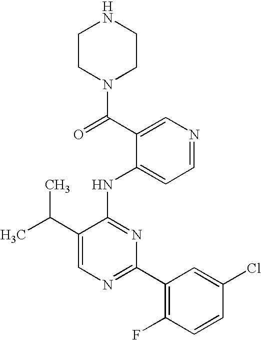 | 455.3, 1.15 | 099 |
| | AutoNom Name: | | |
| | {4-[2-(5-Chloro-2-fluoro-phenyl)-5- | | |
| | isopropyl-pyrimidin-4-ylamino]-pyridin- | | |
| | 3-yl}-piperazin-1-yl-methanone | | |
| 247 | 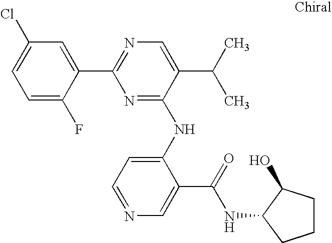 | 470.3, 1.69 | 0.04 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxy-cyclopentyl)-nicotinamide | | |
| 248 | 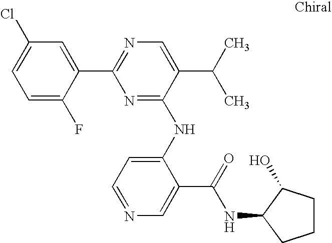 | 470,3, 1.67 | 0.123 |
| | AutoNom Name: | | |
| | 4-[2-(5-Chloro-2-fluoro-phenyl)-5- | | |
| | isopropyl-pyrimidin-4-ylamino]-N-(2- | | |
| | hydroxy-cyclopentyl)-nicotinamide | | |

HPLC conditions: HPLC Column: Merck AGA Chromolith Flash column (25 x 4.6 mm)
HPLC solvents: A: water with 0.1% trifluoroacetic acid. B: acetonitrile with 0.1% trifluoroacetic acid.
Standard Gradient: 5% B to 95% B over 2.5 minutes with a flow rate of 3.0 mL/min.
[HU a[L Alternative Gradient: 5% B to 95% B over 4 minutes at a flow rate of 3.0 mL/min.

1. A compound of formula (1): 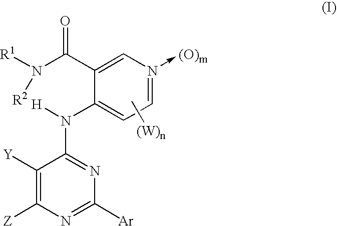
wherein Ar represents an optionally substituted phenyl ring;
Y represents H, halo, NO₂, or an optionally substituted member selected from the group consisting of alkyl, alkenyl, alkynyl, heteroalkyl, heteroalkenyl, heteroalkynyl, aryl, heteroaryl, arylalkyl, heteroarylalkyl, acyl, and heteroacyl,
or Y can be NR₂, wherein each R is independently H or an optionally substituted alkyl, alkenyl, alkynyl, acyl, aryl or arylalkyl group or a heteroform of any of these groups, and wherein two R groups can cyclize to form an optionally substituted 3-8 membered heterocyclic ring;
R¹ represents an optionally substituted group selected from alkyl, heteroalkyl, acyl, alkoxy, alkylamino, heteroacyl, aryl, heteroaryl, arylalkyl, and heteroarylalkyl, where each heteroalkyl, heteroacyl, heteroaryl, and heteroarylalkyl includes one or more heteroatoms selected from O, N, S and P,
provided that R¹ is not a group of the formula —CH₂—CH(OH)—R⁴, where R⁴ is H or an optionally substituted hydrocarbyl group that does not comprise an amine;
R² represents H, or R² represents CH₂ and R¹ and R² cyclize to form an optionally substituted piperidine, morpholine, or piperazine ring, or a pyrrolidine ring substituted with at least one amino or halo substituent;
Z represents H, halo, NO₂, or an optionally substituted member selected from the group consisting of alkyl, alkenyl, alkynyl, heteroalkyl, heteroalkenyl, heteroalkynyl, aryl, heteroaryl, arylalkyl, heteroarylalkyl, acyl, and heteroacyl, or Z is NR₂,
wherein each R is independently H or an optionally substituted alkyl, alkenyl, alkynyl, acyl, heteroacyl, aryl or arylalkyl group or a heteroform of any of these groups;
each W independently represents halo, NR₂, NO₂, CN, CF₃, or an optionally substituted member selected from the group consisting of alkyl, alkenyl, alkynyl, heteroalkyl, heteroalkenyl, heteroalkynyl, aryl, heteroaryl, acyl, heteroacyl, arylalkyl, and heteroarylalkyl,
wherein each R is independently H or an optionally substituted alkyl, alkenyl, alkynyl, acyl, aryl, heteroalkyl or heteroaryl group;
m is 0 or 1;
n is 0-3;
and
(a) Y is selected from the group consisting of a 5-6 membered cyclic amine, OH, F, Cl, Br, and I; or
(b) m is 1; or
(c) R¹ is OH or an optionally substituted alkoxy or an optionally substituted alkylamine, or
(d) R² represents CH₂ and R¹ and R² cyclize to form an optionally substituted piperidine, morpholine, or piperazine ring, or a pyrrolidine ring substituted with at least one amino or halo substituent;
(e) R¹ comprises C—NH₂, a nitrile, a lactam or a lactone ring, or a ketone, or an optionally substituted 4-5 membered cyclic amine; or
(f) R¹ comprises at least two substructures independently selected from the group consisting of:
(1) C—NH—C,
(2) C—OH,
(3) C═O,
(4) P═O,
(5) S═O,
(6) C═N,
(7) a non-cyclic ether oxygen,
(8) a tertiary non-acylated amine;
(9) a 5-6 membered aromatic or heteroaromatic ring,
(10) C—X where X is selected from OH, Cl, and F,
(11) C_{T}—O—R⁴, wherein C_{T} represents a carbon bonded to three other carbon atoms, and R⁴ is H or an optionally substituted hydrocarbyl group, and
(12) an optionally substituted 3 to 8 membered carbocyclic ring; or
(f) R¹ comprises —CH₂)₃—OR⁴ or —CH₂)₃—N(R⁴)₂, wherein each R⁴ is independently H or an optionally substituted hydrocarbyl group;
or a pharmaceutically acceptable salt thereof.

2. The compound of claim 1, wherein Ar is a substituted phenyl.

3. The compound of claim 1, wherein Ar is substituted with 1-2 groups selected from halo, C1-C4 alkyl, CN, CF₃, and C1-C4 alkoxy.

4. The compound of claim 1, wherein n is 0 or 1.

5. The compound of claim 1, wherein Z is H.

6. The compound of claim 5, wherein Y is selected from the group consisting of halo, OH, OR, NR₂, and R, wherein each R is an optionally substituted group selected from C1-C8 alkyl, C1-C8 heteroalkyl, C6-C12 arylalkyl, and C6-C12 heteroarylalkyl,
and where two R groups of NR₂ can optionally cyclize to form 3-8 membered ring containing 1-2 heteroatoms selected from N, O and S.

7. The compound of claim 5, wherein Y is selected from the group consisting of 1-pyrollidinyl, cyclopentyl, F, Cl, Br, I, and OH.

8. The compound of claim 1, wherein R¹ comprises at least one S═O or P═O.

9. The compound of claim 1, wherein R¹ comprises at least one C—NH—C or C—OH.

10. The compound of claim 1, wherein R¹ comprises at least one C═N or C≡N.

11. The compound of claim 1, wherein R¹ comprises at least one C—F or one C—Cl or one tertiary alcohol.

12. The compound of claim 1, wherein R¹ comprises at least one cyclic ether or C═O.

13. The compound of claim 1, wherein R¹ comprises at least one aryl, heteroaryl, lactam, or lactone ring.

14. The compound of claim 9, wherein Z is H.

15. The compound of claim 14, wherein Ar represents phenyl substituted with 1-2 groups selected from halo, CN, CF₃, C1-C4 alkyl, and C1-C4 alkoxy.

16. The compound of claim 15, wherein n is 0 or 1, and W if present is selected from the group consisting of halo, methyl, CF₃, and OMe.

17. The compound of claim 14, wherein Y is selected from the group consisting of halo, C1-C5 alkyl, OH, OR, NR₂, wherein each R is an optionally substituted group independently selected from C1-C8 alkyl, C1-C8 heteroalkyl, and C6-C10 arylalkyl, and wherein two R groups of NR₂ can cyclize to form a 3-8 membered optionally substituted heterocyclic ring containing 1-2 heteroatoms selected from N, O and S as ring members.

18. The compound of claim 15, wherein n is 0.

19. The compound of claim 18, wherein Ar is phenyl substituted with at least one F, Cl or Br.

20. The compound of claim 18, wherein Ar is substituted with at least two halo substituents.

21. The compound of claim 1, wherein m is 1.

22. The compound of claim 1, wherein m is 0.

23. The compound of claim 1, which is any compound in Table 1 for which the Table lists an IC-50.

24. A pharmaceutical composition comprising a compound according to claim 1.

25. Use of a compound according to claim 1 for the treatment of a disorder characterized by an excessive level of TGFβ activity.

26. The use of claim 25, wherein the disorder characterized by an excessive level of TGFβ activity is a fibroproliferative condition.

27. The use of claim 26, wherein the fibroproliferative condition is selected from the group consisting of glomerulonephritis (GN) such as mesangial proliferative GN, immune GN, or crescentic GN, diabetic nephropathy, renal interstitial fibrosis, renal fibrosis in transplant patients receiving cyclosporin, HIV-associated nephropathy, progressive systemic sclerosis, polymyositis, scleroderma, dermatomyositis, eosinophilic fascitis, morphea, vascular disorders associated with the occurrence of Raynaud's syndrome, adult respiratory distress syndrome, chronic obstructive pulmonary disease (COPD), idiopathic pulmonary fibrosis, interstitial pulmonary fibrosis, fibrosis associated with autoimmune disorders such as systemic lupus erythematosus, scleroderma, chemical contact, or allergies, rheumatoid arthritis, and fibroproliferative conditions associated with surgical eye procedures such as retinal reattachment surgery accompanying proliferative vitreoretinopathy, cataract extraction with intraocular lens implantation, or post glaucoma drainage surgery.

28. The use of claim 25, wherein the disorder characterized by an excessive level of TGFβ activity is cancer.

29. The use of claim 28, wherein the cancer is brain cancer, pancreatic cancer, or breast cancer or glioma.

30. (canceled)

31. (canceled)
